(19) United States
(12) Patent Application Publication
(10) Pub. No.: US 2025/0034234 A1
(43) Pub. Date: Jan. 30, 2025

(54) PREDICTING COVID-19 ANTIBODIES AMONG SURVIVORS WITH DEEP RNA SEQUENCING

(71) Applicant: Rhode Island Hospital, Providence, RI (US)

(72) Inventors: Sean F. Monaghan, East Greenwich, RI (US); Alger M. Fredericks, East Providence, RI (US); Gerard J. Nau, Sharon, MA (US)

(21) Appl. No.: 18/708,467

(22) PCT Filed: Nov. 9, 2022
(86) PCT No.: PCT/US2022/079552

§ 371 Date: May 8, 2024

Related U.S. Application Data
(60) Provisional application No. 63/378,366, filed on Oct. 4, 2022.
(60) Provisional application No. 63/378,365, filed on Oct. 4, 2022.
(60) Provisional application No. 63/277,475, filed on Nov. 9, 2021.

(51) Int. Cl. C07K 16/10; C12N 15/10; C12Q 1/689; G01N 33/68

(52) U.S. Cl. CPC C07K 16/1003; C12N 15/1096; C12Q 1/689; G01N 33/6854; C07K 2317/565; G01N 2333/165

(57) ABSTRACT

The invention provides a method for treating COVID-19 patients. Therapeutic antibodies of certain types of antibodies to COVID-19 are identified early by targeted diagnostic testing can be given to other COVID-19 patients to enhance their recovery. This invention also identifies bacterial pathogens, particularly those that are resistant to antibiotics, faster. Data from deep RNA sequencing of human blood is used to create a faster diagnostic test for infections and associated antimicrobial resistance. This invention thus also allows for appropriate antibiotic treatment at an earlier time point. The invention further provides improvements to RNA sequencing that can make RNA sequencing useful for diagnostic and therapeutic methods of treating sepsis. RNA sequencing can identify bacterial pathogens directly from the blood of patients with those infections.

### REFERENCE TO RELATED APPLICATIONS

This application is a national phase filing under 35 U.S.C. § 371 of International Application No. PCT/US2022/079552 filed Nov. 9, 2022, which claims priority from U.S. Provisional Patent Application No. 63/277,475 filed Nov. 9, 2021, U.S. Provisional Patent Application No. 63/378,365 filed Oct. 4, 2022 and U.S. Provisional Patent Application No. 63/378,366 filed Oct. 4, 2022, the entire contents of which is hereby incorporated by reference herein.

### STATEMENT REGARDING FEDERALLY SPONSORED RESEARCH OR DEVELOPMENT

This invention was made with government support under T32 HL134625, P20 GM103652, and R35 GM142638 awarded by National Institutes of Health. The government has certain rights in the invention.

### TECHNICAL FIELD OF THE INVENTION

This invention generally relates to chemical analysis of biological material, using nucleic acid products used in the analysis of nucleic acids, e.g., primers or probes for diseases caused by alterations of genetic material. This invention also relates particularly to COVID-19, to RNA sequencings, to antibody identification, and to antibody function.

### SEQUENCE LISTING

The instant application contains a Sequence Listing which has been submitted electronically in XML format and is hereby incorporated by reference in its entirety. Said XML copy, created on Feb. 10, 2023, is named 405002-529001WO_SL.xml and is 59,603,000 bytes in size.

### BACKGROUND OF THE INVENTION

Severe acute respiratory syndrome coronavirus 2 (SARS-CoV-2) causing coronavirus disease 2019 (COVID-19) continues to cause critical illnesses requiring intensive care unit (ICU) admission. These ICU admissions are currently driven by the presence of COVID-19 variants. Krause et al., New England Journal of Medicine, 385, 179-86 (2021). Few direct treatments against COVID-19 are available. Willyard, Nature (2021). With COVID-19 variants comes further potential for escape from current treatments and vaccines. Acevedo et al., medRxiv, 2021.06.28.21259673 (2021); Starr et al., SARS-CoV-2 RBD antibodies that maximize breadth and resistance to escape. Nature (2021).

Quickly identifying novel antibodies to target the new variants could aid in the care of these patients, reducing viral load, and preventing hypoxemia. This identification is important for two reasons: First, most antibodies currently target the SARS-CoV-2 spike protein receptor-binding domain (RBD) and mutations in this region are present in variants. Starr et al., Science, 371, 850-4 (2021). Second, the de novo discovery and testing of antibodies is a very time-consuming process. Weinreich et al., New England Journal of Medicine, 384, 238-51 (2020).

The inventors previously proposed using RNA sequencing data from critically ill patients to identify antibodies for other diseases. Typically, only RNA sequencing data that aligns to the organism of interest is analyzed. However, some groups recently began analyzing data that is unmapped to an organism of interest. Mangul et al., Genome Biology, 19, 36 (2018). The inventors previously showed many uses for unmapped data. Monaghan et al., medRxiv (2021).

Variants of severe acute respiratory syndrome coronavirus 2 (SARS-CoV-2) could be ideal candidates for treatment by novel antibodies. However, development and testing of antibodies depends upon the presence of a COVID-19 variant and knowledge of the genetic sequence. Thus, there is a need in the biomedical art for deep RNA sequencing in patients with COVID-19 to elucidate immune antibodies produced during the course of the disease.

As a viral infection, COVID-19 can lead to sepsis. People with severe COVID-19 may experience symptoms such as fever and chills, difficulty breathing, pain or discomfort, and confusion. These are also common signs of sepsis, which happens when an infection causes your body's immune system to mount an extreme response. This excessive response can lead to tissue damage, organ failure, and even death if not treated promptly. See Based on Science, What is the connection between COVID-19 and sepsis? National Academies (Feb. 14, 2022). Notably, both COVID-19 patients and sepsis patients experience consumptive thrombocytopenia, haemolytic anaemia, vascular microthrombosis, multi-organ dysfunction syndrome, coagulopathy, septic shock, respiratory failure, fever, leukopenia, hypotension, leukocytosis, high cytokine production and high predisposition to opportunistic infections. Olwal et al., Front. Immunol. (February 2021).

Infection in any part of the body can cause sepsis. This includes infections of the lung (e.g., pneumonia), genitourinary tract (e.g., UTI), or abdomen (e.g., appendicitis).

RNA sequencing can be a valuable tool in personalizing the care of sepsis patients. Currently, RNA sequencing is done by first creating a DNA library. This conversion of DNA takes time. Direct RNA sequencing would allow faster processing times. There is a need in the biomedical art for diagnostic tests for diagnosing and treating sepsis.

### SUMMARY OF THE INVENTION

The invention provides a method and computational tool to identify antibodies produced by COVID-19 patients, such as patients being treated in a hospital intensive care unit. The invention uses the proposed structural classification of COVID-19 antibodies, such as described by Barnes et al., Nature, 588, 682-7 (2020), to categorize potential efficacious antibodies and provide a possible rationale for this effect.

In a first embodiment, the antibodies are generated by patients that survived severe COVID-19, which can inform future antibody or vaccine development. Patients that survive intensive care unit admission because of COVID-19 have a different antibody response as compared to non-survivors.

In a second embodiment, patients lacking certain types of antibodies to COVID-19 are identified early by targeted diagnostic testing.

In a third embodiment, the invention provides a method for treating COVID-19 patients. Therapeutic antibodies of certain types of antibodies to COVID-19 are identified early by targeted diagnostic testing can be given to other COVID-19 patients to enhance their recovery. In fifth embodiment, a cocktail of antibodies from TABLE 6 is used for treating COVID-19 patients, as a treatment in addition to COVID-19 vaccinations. In a sixth embodiment, a cocktail of antibodies from TABLE A4 is used for treating COVID-19 patients.

In one aspect, when looking at COVID-19 patients across several time points, the inventors identified what was shown to be a critical intervention point in patients with reduced levels of COVID-19 antibodies.

The invention also provides molecular diagnostic tests to overcome the limitations of conventional microbiological approaches for diagnosing and treating sepsis.

In a fourth embodiment and as a proof of principle, the inventors develop polymerase chain reaction (PCR) diagnostic tests for four common bacteria: Staphylococcus aureus, extra-intestinal Escherichia coli, Pseudomonas aeruginosa, and Haemophilus influenzae.

In a fifth embodiment, the inventors create tests of clinically relevant resistance genes associated with these four pathogens. These bacteria are pathogens of interest in the request for applications (RFA-AI-22-010) for bacteremia (S. aureus, E. coli, P. aeruginosa) and pneumonia (S. aureus, P. aeruginosa, H. influenzae). These pathogens are also the most common causes of bacteremia and hospital-acquired pneumonia at Rhode Island Hospital. These four pathogens have antimicrobial resistance attributable to specific genes requiring antibiotic management changes. These four pathogens are the top organisms that cause death due to resistance. Global burden of bacterial antimicrobial resistance in 2019 (2022).

In a sixth embodiment, the invention provides tests of clinically relevant resistance genes associated with other pathogens that cause sepsis.

In a seventh embodiment, the invention provides a diagnostic PCR test based on bacterial RNA. PCR tests were previously developed for respiratory pathogens. See Covert, Bashore, Edds, & Lewis (2021). PCR tests were also developed for identifying the DNA of bacteria like Staphylococcus aureus in targeted sites. Palavecino (2020). Pathogen identification was accomplished by sequencing cell-free DNA from blood. Camargo et al. (2019).

By contrast, in the diagnostic PCR test of the invention, the most abundant RNA targets are selected from the blood of patients with these infections, making the approach more sensitive than single-copy DNA targets. Antibiotic resistance correlates closely with gene expression. The risk of RNA degradation is significantly mitigated by stabilizing RNA. Because the targets are derived from RNA sequencing data, those RNAs are abundant and measurable in patients with infection.

In another aspect, the unmapped RNA reads from patients with infections that align with pathogens can inform a better diagnostic test. PCR targets for diagnostics come from a data set created from deep sequencing (>100 million reads) of the blood of patients with bacteremia or pneumonia. Pathogen identification is performed with standard culture techniques. The RNA sequences from the pathogens are typically discarded in transcription analysis because they would not align with the human genome. These “unmapped reads” are being identified in the blood of patients and aligned to a custom “genome” derived from them pathogens of interest to identify the causative organism. RNAs that align with resistance genes are also identified.

In an eighth embodiment (A1a), the invention provides a direct from blood, without the need for culture, reverse transcriptase polymerase chain reaction (RT-qPCR) test for bacteria causing bacteremia, specifically S. aureus, E. coli, P. aeruginosa, based on the RNA identified in patients with bacteremia caused by these organisms.

In a ninth embodiment (A1b), the invention provides a method to validate these RT-qPCR tests in samples from patients with and without bacteremia.

In a tenth embodiment (A2a), the invention provides a direct from blood, without the need for culture, reverse transcriptase polymerase chain reaction (RT-qPCR) test for bacteria causing pneumonia, specifically S. aureus, P. aeruginosa, H. influenzae, based on the RNA identified in patients with pneumonia caused by these organisms.

In an eleventh embodiment (A2b), the invention provides a method to validate these RT-qPCR tests in samples from patients with and without pneumonia.

In a twelfth embodiment (A3a), using the RNA from patients with infections, the invention provides an RT-PCR for the most common resistance genes expressed that would influence treatment for S. aureus, E. coli, P. aeruginosa, and H. influenzae.

In a thirteenth embodiment (A3b), the invention provides a method to validate these PCR tests for resistance genes in samples from patients with and without infections.

In a fourteenth embodiment, the invention provides a direct from blood RT-PCR test for bacteremia caused by S. aureus, E. coli, and P. aeruginosa without the need for culture with phenotypic microbial resistance identification.

In a fifteenth embodiment, the invention provides a direct from blood RT-PCR test for pneumonia caused by S. aureus, P. aeruginosa, and H. influenzae without the need for culture with phenotypic microbial resistance identification.

In a sixteenth embodiment, the invention provides that all tests can have a result in fewer than four hours from the time of sample collection.

In a seventeenth embodiment, the invention provides the ability to standardize and scale these tests for use in a clinical microbiology setting. See TABLE 3 below.

In another aspect, the discovery of genes, antibodies, and methods of the invention is a hypothesis-free method of investigation. See, Glass & Hall, A brief history of the hypothesis. Cell, Volume 134, Issue 3, pages 378-381 (Aug. 8, 2008); Glass, A critique of the hypothesis, and a defense of the question, as a framework for experimentation. Clinical Chemistry, Volume 56, Issue 7, pages 1080-1085 (Jul. 1, 2010).

In another aspect, the invention provides a direct form blood PCR panel (e.g., using the top twelve pathogens) that identifies the pathogens and resistance profile faster (fewer than four hours) than current bacteremia and hospital-acquired pneumonia techniques. This invention translates deep RNA sequencing data into a product: a rapid PCR to identify S. aureus, E. coli, P. aeruginosa, and H. influenza and potential resistance genes without the need for culture or specimens other than blood.

The following factors are improvements to RNA sequencing that can make RNA sequencing useful for diagnostic and therapeutic methods of treating sepsis. Optimizing the factors is being done in collaboration with an industry partner that provides sample and assay technologies for molecular diagnostics. The objective is to establish a standard set of testing conditions to be used on the system of the industry partner. These improvements are supported by the literature and preliminary data indicating that RNA sequencing can identify bacterial pathogens directly from the blood of patients with those infections.

First factor, decrease the cost of RNA collection tubes. The PAXgene Blood RNA Tubes (QIAGEN, Germantown, MD, USA; Cat. No./ID: 762165) are used for in vitro diagnostic testing (IVD). These tubes currently cost about $10/tube. If they are to be used with all blood cultures (˜30,000 at a single academic medical center), the cost would need to be reduced to $0.10/tube.

Second factor, increase the speed of RNA sequencing on machines. An Illumina machine takes about eighteen hours to obtain 350 million reads, not including processing time. For RNA sequencing to be impactful with sepsis, the RNA sequencing results are needed in <4 hours so effective antibiotic treatment can begin for the sepsis patients.

More recently, a new Illumina Machine (NovaSeq X+) was announced. This Illumina machine should be able to take only thirteen hours to obtain 1.6 billion reads, not including processing time. While this is an improvement, the goal is to make sequencing fast enough for sepsis. An ideal machine for me would get 100 million reads in an hour.

Third factor, increase the processing of RNA data. The inventors are processing 100 million data points. Physicians and medical laboratory personnel need to process these data within the <4-hour time frame for effective sepsis treatment results.

The commonly identified and organism-specific sequences (for S. aureus, E. coli, P. aeruginosa, and H. influenza) are the template for designing oligonucleotide primers for RT-qPCR tests. Future efforts to expand the diagnostic test to other pathogens may require RNA sequencing from patients with pneumonia due to other pathogens.

Fourth factor, direct RNA sequencing should be done for these assays. Currently, RNA sequencing is done by first creating a DNA library. This conversion of RNA to DNA takes time. Direct RNA sequencing allows faster processing times, to be completed in the 4-hour time frame. Focusing on RNA rather than DNA improves phenotypic correlation with antimicrobial resistance.

RNA sequencing can be a valuable tool in personalizing the care of sepsis patients. With these advances, this tool will be used by clinicians in the Intensive Care Unit caring for sepsis patients. The improvements described above should expand the technology from the research laboratory to the clinical microbiology laboratory.

The improvements listed above enable a direct from blood, without the need for culture, reverse transcriptase polymerase chain reaction (RT-qPCR) test for bacteria and methods to validate these RT-qPCR tests in samples from patients. RT-qPCR tests are useful in the diagnosis and treatment of sepsis. The improvements listed above also enable methods for combatting the scourge of drug-resistant bacteria.

### BRIEF DESCRIPTION OF THE DRAWINGS

For illustration, some embodiments of the invention are shown in the drawings described below. Like numerals in the drawings indicate like elements throughout. The invention is not limited to the precise arrangements, dimensions, and instruments shown.

FIG. 1 is a chart showing the total counts for CDR3 regions that aligned to the antibody C135 in survivors vs. non-survivors. Survivors had significantly more than non-survivors (15,059.4 vs. 1,412.7, p=0.016).

FIG. 2 is a pair of images showing the binding of C135 antibody and ACE2 to the SARS-CoV-2 spike trimer RBD. (A) Structure of Class 3 neutralizing antibody (C135) bound to the RBD of the spike protein (PDB: 7K8Z) with a cutout highlighting the intermolecular interactions (hydrogen bonds) between the light chain CDR3 (light blue) and the Spike RBD (purple). (B) Spike protein trimer with its RBD (purple) bound to the C135 Class 3 antibody (blue) and ACE2 (yellow), highlighting the distinct binding sites on opposite sides of the target RBD.

FIG. 3 shows the RT-qPCR validation of sequencing results. cDNA from patient RNA was tested for the SARS-CoV-2 N gene using real-time PCR. FIG. 3A is a map of the N gene showing the location of the peak of sequencing reads (red box) and primers directed to the peak or elsewhere in the gene (“off peak). FIG. 3B is a bar graph showing the relative expression level of peak and off-peak sequences in fifteen critically ill COVID-19 patients. The peak sequence is shown in red and off-peak in black.

TABLE A1 in the Appendix lists patient-derived light chain CDR3 sequences that were aligned to known CDR3 regions of previously identified neutralizing antibodies. CDR3 sequences with similar primary sequences to the CDR3 classifications were placed into categories. Table discloses SEQ ID NOS 54-64, 64-67, 65, 68, 57, and 69-71, respectively, in order of appearance.

TABLE A2 in the Appendix lists the antibodies Identified on Day 0 in the intensive care unit. Table discloses SEQ ID NOS 72-747, 8, 748-1704, 11, 1705-1714, 24, 1715-2269, 910, 2270-2295, 7, 2296-2330, 2018, 2331-2403, 26, 2404-2435, 2001, 2436-2455, 1511, 2456-2750, 33, 2751-2810, 356, 2811-2955, 527, 2956-3021, 1654, 3022-3137, 866, 3138-3259, 898, 3260-3473, 18, 3474-3523, 2285, 3524-3687, 1447, 3688-3722, 27, 3723-3802, 5, 3803-3875, 2018, 3876-3957, 2280, 3958-4114, 14, 4115-4151, 28, 4152-4170, 1626, 4171-4213, 2092, 4214-4475, 3550, 4476-4678, 6, 4679-4701, 2392, 4702-4738, 2280, 4739-4879, 1882, 4880-4935, 2018, 4936-4987, 20, 4988-5151, 19, 5152-5374, 467, 5375-5440, 2957, 5441-5452, 50, 5453-5502, 2783, 5503-5536, 2280, 5537, 38, 5538-5578, 2485, 5579-5586, 1555, 5587-5753, 4396, 5754-5762, 48, 5763-5778, 3563, 5779-5815, 467, 5816-5827, 235, 5828-5847, 2481, 5848-5962, 46, 2280, 5963-6045, 1263, 6046-6067, 2544, 6068-6109, 49, 6110-6148, 2426, 6149-6198, 898, 6199-6250, 23, 6251-6311, 235, 6312-6316, 898, 6317-6340, 5871, 6341-6355, 4691, 6356-6489, 261, 6490-6509, 6094, 6510-6679, 21, 6680-6789, 2226, 6790-6946, 15, 6947-6958, 9, 6959-6968, 856, 6969-7209, 4, 7210-7226, 6045, 7227-7284, 3867, 7285-7367, 2280, 7368-7518, 5614, 7519-7586, 2, 7587, 6022, 7588-7616, 1626, 7617-7718, 5359, 7719-8007, 1955, 8008-8067, 2426, 8068-8152, 53, 8153-8319, 39, 8320-8447, 45, 8448-8464, 982, 8465-8491, 898, 8492-8660, 4396, 8661-8814, 4826, 8815-9083, 3960, 9084-9141, 7411, 9142-9359, 2481, 9360-9578, 920, 9579-9666, 856, 9667-9681, 5834, 898, 9682-9712, 5903, 9713-9969, 25, 9970-9972, 2947, 9973-10005, 2564, 2426, 10006-10034, 6206, 10035-10083, 632, 10084-10097, 931, 10098-10147, 3, 10148-10153, 6186, 10154-10284, 2402, 10285-10289, 866, 10290-10546, 527, 10547-10823, 5906, 10824-10923, 2018, 10924-10931, 99, 10932-10994, 13, 10995-11007, 6148, 11008-11022, 916, 11023-11052, 3867, 11053-11193, 96, 11194-11493, 2559, 11494-11550, 9419, 11551-11690, 5356, 11691-11701, 9539, 11702-11790, 2356, 11791-11858, 2297, 11859-11941, 467, 11942-12194, 36, 12195-12215, 2437, 12216-12328, 2570, 12329-12333, 2580, 12334-12855, 2366, 12856-12953, 5825, 12954-13019, 5150, 13020-13140, 2419, 13141-13334, 2493, 13335-13391, 2426, 13392-13420, 10039, 13421-13424, 594, 13425-13442, 10075, 13443-13715, 10479, 13716-13765, 6148, 13766-13831, 2498, 13832-13873, 2392, 13874-13905, 1399, 13906-13985, 2018, 13986-14267, 5527, 14268-14305, 2559, 14306-14398, 2423, 14399-14681, 43, 14682-14762, 2564, 14763-14929, 2387, 14930-14946, 2402, 14947-15117, 2464, 15118-15161, 2451, 15162-15355, 13, 5903, 15356-15489, 10589, 15490-15579, 2335, 15580-15855, 23, 15856-15922, 235, 15923-15993, 9802, 15994-16241, 2481, 16242-16624, 4826, 16625-16742, 3867, 16743-16891, 2372, 16892-17025, 898, 17026-17151, 23, 17152-17246, 467, 17247-17250, 34, 17251-17270, 5834, 17271-17305, 6206, 17306-17308, 5903, 17309-17393, 5908, 17394-17448, 9956, 17449-17453, 11893, 17454-17580, 2426, 17581-17612, 2947, 17613-17639, 2559, 17640-17668, 2993, 17669-17769, 3162, 17770-17867, 15845, 17868-18157, 12626, 18158-18410, 6991, 18411-18751, 1447, 18752-18770, 11104, 18771-18887, 11578, 18888-18898, 16690, 18899-19238, 10879, 19239-19271, 15516, 19272-19273, 4016, 19274-19367, 4061, 19368-19411, 3596, 19412-19542, 2018, 19543-19600, 2485, 19601-19665, 3686, 19666-19690, 47, 19691-19716, 5575, 19717-19721, 12985, 19722-19854, 15154, 19855-19976, 14119, 19977-20019, 13191, 20020-20188, 37, 20189-20253, 15845, 20254-20339, 12772, 20340-20357, 23, 20358-20374, 467, 20375-20401, 2426, 20402-20481, 11894, 20482-20490, 12001, 20491-20517, 14041, 20518-20567, 6206, 20568, 10032, 20569-20691, 2559, 20692-20745, 10153, 20746-20844, 14903, 20845-20877, 5060, 20878-20892, 12217, 20893-21016, 17845, 21017-21107, 235, 21108-21143, 5039, 21144-21317, 10545, 21318-21319, 12174, 21320-21338, 6148, 21339-21379, 6991, 21380-21463, 42, 21464-21644, 19802, 21645-21764, 13411, 21765, 4826, 21766-21780, 11104, 21781-21820, 52, 21821-21857, 2018, 21858-21889, 5575, 21890-21910, 18502, 21911-21947, 5903, 21948-22039, 3867, 22040-22052, 51, 22053-22106, 41, 22107-22364, 14192, 22365-22556, 5527, 22557-22768, 5150, 22769-22814, 2481, 22815-23012, 15278, 23013-23026, 6186, 23027, 15845, 23028-23033, 5506, 23034-23066, 21706, 23067-23090, 2481, 23091-23104, 21673, 23105-23135, 17326, 23136-23170, 6148, 23171, 19793, 23172-23173, 4826, 23174-23181, 18763, 23182-23194, 5527, 23195-23196, 6206, 23197-23201, 44, 23202-23214, 5903, 23215-23250, 21871, 23251-23264, 5575, 23265-23278, 16048, 23279-23296, 13, 23297-23323, 15743, 23324-23375, 2426, 23376-23382, 19741, 23383, 35, 23384-23412, 5678, 23413-23512, 16529, 23513-23725, 5834, 23726-23786, 23300, 23787-23789, 23111, 23790-23842, 9750, 23843-23876, 11852, 23877-23924, 356, 23925-24006, 5980, 24007-24207, 2426, 24208-24319, 2559, 24320-24366, 2578, 24367-24405, 6206, 24406-24446, 12039, 24447-24623, 17326, 24624-24712, 5795, 24713-24724, 11912, 24725-24796, 2481, 24797-24853, 14101, 24854-24971, 5929, 24972-25200, 12278, 25201-25383, 5825, 25384-25405, 31, 25406-25409, 6148, 25410-25426, 22291, 25427-25469, 5835, 25470-25653, 23020, 25654-25661, 6198, 25662-25665, 16511, 25666-25756, 5724, 25757-25815, 5854, 25816-25973, 13941, 25974-26077, 30, 26078-26238, 5903, 26239-26504, 15845, 26505-26566, 15548, 26567-26697, 7411, 26698-26750, 29, 26751-26795, 22383, 26796-26967, 16512, 26968-27082, 18265, 27083-27098, 18976, 27099-27240, 11515, 27241-27297, 23312, 27298-27354, 19702, 27355-27409, 6145, 27410-27549, 23163, 23677, 27550-27562, 18448, 27563-27566, 5575, 27567-27582, 22, 27583-27624, 16503, 27625-27630, 11104, 27631, 6186, 27632, 22291, 27633-27672, 10, 27673-27677, 856, 27678-27680, 16844, 2957, 27681-27693, 5834-5835, 27694-27698, 11274, 27699-27702, 2481, 27703-27711, 21673, 27712-27738, 23344, 27739-27745, 12446, 7432, 27746-27762, 14746, 27763-27787, 898, 27788-27818, 6206, 27819-27830, 11449, 27831-27832, 15317, 27833-27855, 2426, 14610, 27856-27872, 16830, 27873-27882, 2559, 27883-27887, 2493, 27888, 20359, 27889-27938, 26311, 27939-27958, 23876, 27959-27960, 3867, 27961-27972, 6145, 27973-27975, 9603, 27976-27981, 26402, 27982-27995, 17134, 27996-28008, 6148, 28009-28020, 26841, and 28021-28042, respectively, in order of appearance.

TABLE A3 in the Appendix is in the Appendix lists the antibodies Identified on Day 3 in the intensive care unit. Table discloses SEQ ID NOS 28043-28061, 2256, 28062-28078, 92, 28079-28083, 96, 1933, 28084-28096, 2147, 28097-28102, 1163, 28103-28115, 5041, 28116-28118, 469, 28119-28120, 123, 920, 28121-28156, 1025, 28157-28172, 168, 28173-28174, 84, 28175-28181, 434, 28182, 172, 28183-28195, 279, 28196-28214, 203, 28215-28228, 746, 28229-28236, 1271, 28237-28246, 230, 28247-28258, 1672, 28259-28263, 1577, 243, 28264, 246, 28265-28267, 248, 28268-28278, 88, 28279-28281, 2247, 4818, 28282-28297, 261, 28298-28300, 1590, 1568, 28301-28324, 1287, 28325-28334, 290, 28335-28346, 297, 28347-28372, 1420, 28373-28377, 1106, 319, 28378-28389, 1329, 28390-28397, 1107, 28398-28403, 193, 28404, 1556, 28405-28408, 9776, 28409-28422, 2175, 28423-28424, 1995, 28425-28464, 370, 28465-28467, 611, 28468-28472, 741, 28473-28492, 385, 1611, 28493-28503, 22502, 28504-28505, 398, 28506-28517, 751, 28518-28521, 1334, 28522-28544, 422, 28545-28549, 425, 28550-28555, 862, 28556-28562, 138, 1925, 28563-28574, 441, 28575-28583, 226, 28584-28585, 448, 28586-28589, 451, 5033, 539, 28590-28595, 834, 17536, 28596-28608, 1527, 28609-28610, 461, 28611-28623, 467, 28624-28643, 479, 28644-28646, 481, 28647-28648, 1082, 318, 28649-28669, 1944, 28670, 1538, 28671-28681, 1953, 28682-28684, 1144, 28685, 508, 28686-28703, 536, 28704-28707, 527, 28708-28714, 492, 28715-28717, 1863, 28718-28721, 672, 28722, 538, 28723-28732, 167, 28733-28740, 1619, 28741-28742, 549, 28743-28745, 981, 28746-28829, 599, 28830-28833, 605, 28834-28853, 505, 28854-28862, 680, 28863-28874, 1226, 28875-28878, 635, 28879-28890, 643, 28891-28894, 768, 28895-28902, 1298, 28903-28931, 666, 28932-28933, 671, 28934-28946, 867, 28947-28948, 681, 28949-28953, 1985, 28954, 180, 28955-28963, 603, 28964-28969, 359, 28970-28973, 2234, 1883, 28974-28986, 831, 28987-28989, 710, 28990-28991, 1231, 28992-28997, 1179, 28998, 1743, 28999-29005, 1839, 725, 29006-29008, 727, 29009-29052, 3148, 29053-29059, 2032, 29060-29067, 730, 29068-29083, 780, 29084-29095, 430, 29096-29100, 793-794, 29101-29110, 803, 29111-29116, 2094, 29117-29132, 207, 29133-29134, 571, 29135-29138, 1845, 29139-29152, 2157, 29153-29158, 1687, 29159-29160, 827, 29161-29168, 212, 859, 29169-29189, 306, 1528, 29190-29222, 866, 1324, 29223-29226, 870, 29227-29254, 335, 29255, 886-887, 29256-29264, 895, 29265-29266, 898, 29267-29284, 909, 29285-29288, 913, 29289, 692, 29290, 23211, 29291-29314, 1322, 29315-29321, 769, 29322-29328, 940, 29329-29340, 946, 29341-29349, 951, 29350-29355, 313, 29356-29365, 962, 29366-29383, 975, 29384-29385, 2184, 29386-29397, 2036, 29398-29399, 1095, 29400-29402, 992, 29403-29421, 1005, 29422-29426, 1241, 29427-29429, 83, 29430-29435, 1015, 29436-29442, 966, 29443-29465, 1038, 29466-29469, 1440, 29470-29485, 113, 29486-29490, 220, 29491-29496, 1058, 29497, 715, 29498-29501, 234, 29502-29508, 1070, 29509-29510, 1661, 29511-29521, 1087, 29522, 1301, 29523-29524, 1666, 29525-29531, 2230, 29532-29536, 559, 29537, 285, 29538-29567, 2211, 29568-29610, 1151, 29611-29618, 2082, 29619-29630, 1366, 1792, 29631-29648, 99, 29649-29650, 1406, 29651-29662, 1371, 29663-29715, 1392, 1180, 29716-29721, 22767, 29722-29724, 1227, 29725, 273, 29726-29734, 1059, 1232, 29735-29742, 853, 29743-29754, 1242, 29755-29767, 1249, 29768, 1846, 29769-29782, 1832, 29783-29796, 1739, 482, 29797-29799, 1272, 29800-29819, 287, 29820-29846, 20697, 29847-29852, 1777, 29853-29866, 1039, 29867-29870, 1843, 29871, 2090, 29872-29876, 1862, 29877-29882, 1318, 29883-29889, 590, 29890-29920, 1338, 29921-29940, 1507, 29941-29960, 1368, 29961-29982, 304, 29983-29991, 722, 29992-29996, 1497, 29997-30000, 1267, 30001-30002, 1704, 30003-30011, 1399, 30012, 1393, 30013-30026, 1411, 30027-30072, 973, 30073, 1449, 30074-30075, 1638, 1641, 30076-30088, 1063, 30089-30093, 2117, 30094, 1467, 30095-30101, 321, 30102-30107, 1477, 30108-30125, 1045, 30126-30127, 1489, 443, 30128-30130, 2003, 30131-30132, 1494, 30133-30149, 1589, 30150-30157, 1506, 30158-30168, 1511, 30169-30171, 1097, 30172-30177, 1518-1519, 30178-30193, 20359, 30194-30196, 1531, 701, 30197-30224, 1543, 30225-30256, 1555, 30257-30286, 597, 30287-30295, 637, 30296, 1592, 30297-30318, 1608, 30319, 1048, 30320-30330, 1604, 30331-30332, 86, 449, 30333-30338, 1627, 30339-30358, 1295, 30359-30369, 1647, 30370-30396, 1286, 30397-30403, 707, 30404-30407, 1679, 30408-30411, 1684, 30412-30422, 1753, 30423-30426, 1691, 30427-30436, 495, 1778, 30437-30457, 1705, 30458-30470, 5085, 30471-30472, 1697, 30473-30476, 4384, 30477-30483, 1725, 30484-30489, 1730, 30490-30498, 1823, 30499-30507, 731, 30508-30534, 1044, 30535-30545, 1779, 30546, 1478, 1495, 30547, 310, 30548, 1036, 30549-30550, 1784, 30551-30553, 1978, 30554-30561, 1789, 30562-30563, 1790, 30564-30573, 22234, 30574-30581, 1585, 30582-30584, 1793-1794, 30585-30601, 1539, 30602-30613, 1562, 1667, 30614-30616, 1814, 30617, 1815, 30618, 763, 30619-30623, 1821, 30624-30627, 1309, 30628-30636, 1840, 30637-30657, 904, 30658-30694, 630, 30695-30708, 1385, 1866, 30709-30720, 5825, 30721-30728, 2201, 30729-30736, 1882, 30737-30739, 1560, 30740-30741, 1244, 30742-30757, 1853, 30758-30779, 1902, 30780, 1503, 30781-30783, 169, 30784-30794, 144, 30795-30798, 1914, 30799-30819, 1928, 30820-30824, 754, 30825-30831, 1932, 30832-30842, 1938, 30843-30865, 1529, 30866-30879, 1917, 30880-30892, 2030, 30893-30896, 1756, 30897-30903, 541, 30904-30907, 204, 30908-30916, 843, 30917-30928, 1597, 30929-30930, 1032, 30931-30936, 19512, 30937-30944, 1802, 30945, 222, 30946-30969, 2015, 4927, 30970, 371, 30971-30972, 758, 30973-30994, 2028, 30995-31000, 963, 31001, 2113, 31002-31009, 2039, 31010-31028, 2051, 31029-31045, 923, 31046, 694, 31047-31055, 2215, 31056-31064, 236, 31065-31074, 1268, 31075-31081, 146, 31082-31103, 1360, 2093, 31104-31108, 2097, 31109-31115, 2107, 31116-31132, 2016, 31133-31143, 2131, 31144, 2133, 31145-31168, 1171, 31169-31173, 2149, 31174-31188, 2161, 31189, 2162, 31190-31212, 820, 31213-31221, 3596, 31222-31232, 5654, 31233-31238, 1270, 31239-31258, 2207, 424, 31259-31265, 1280, 31266-31267, 873, 31268-31269, 2216, 31270-31280, 1237, 2075, 31281-31285, 2226-2227, 31286-31287, 198, 31288-31305, 2245, 1615, 31306-31322, 2258, 31323, 4239, 31324-31349, 4272, 31350-31354, 3779, 31355-31358, 3617, 31359-31362, 3646, 31363-31370, 3029, 31371, 9508, 31372-31406, 2673, 31407-31425, 2680, 31426-31445, 2700, 31446-31456, 2705, 31457-31458, 6287, 31459-31466, 3322, 31467-31472, 2713, 31473, 4152, 2716, 31474-31481, 3998, 4026, 31482-31486, 2727, 31487-31492, 2731, 31493-31499, 2957, 31500-31529, 2745, 31530, 3685, 31531-31557, 4109, 31558-31570, 15951, 31571-31572, 4135, 31573-31582, 2771, 31583-31589, 3427, 4311, 2779, 31590-31603, 2785, 31604-31605, 4271, 31606-31618, 2872, 31619-31627, 3310, 2800, 31628-31653, 2818, 31654-31675, 3260, 31676-31677, 2685, 31678-31688, 3106, 31689-31700, 2850, 31701-31717, 2974, 31718-31739, 4195, 31740-31761, 3712, 3378, 31762-31784, 467, 31785-31791, 2848, 31792-31807, 3735, 31808-31815, 2920, 31816-31829, 3472, 31830-31835, 3142, 2932, 31836-31840, 2909, 31841-31861, 2980, 31862-31885, 2970, 4255, 31886-31900, 3999, 31901, 2981, 31902-31905, 19099, 31906, 2988, 31907-31916, 2997, 31917-31925, 3874, 31926-31948, 3018, 594, 31949-31951, 3680, 31952, 3210, 31953-31955, 3022, 31956, 3901, 3842, 31957-31969, 3033, 31970-32027, 17782, 32028-32053, 3065, 32054-32058, 2892, 32059-32068, 3509, 32069-32075, 3563, 32076-32080, 3088, 32081-32085, 3093, 32086-32087, 3897, 32088-32157, 4139, 3737, 32158-32162, 3141, 32163-32166, 3143, 32167-32198, 4166, 32199-32219, 3169, 32220-32241, 3476, 32242, 3654, 2625, 32243-32257, 3193, 32258, 3195, 32259-32270, 3204, 32271-32281, 3222, 32282-32293, 3231, 32294-32305, 4191, 32306-32351, 17162, 32352, 898, 32353-32367, 3269, 32368-32381, 3361, 32382-32412, 3297, 32413-32421, 3791, 32422-32429, 3305, 32430-32438, 2765, 32439-32459, 3578, 32460-32463, 3326, 32464-32471, 3332, 32472-32490, 3583, 3453, 32491-32526, 3388, 32527-32537, 3395, 21673, 32538, 3396, 32539-32541, 3808, 32542-32551, 3403, 3320, 32552-32559, 3408, 32560-32564, 3570, 32565-32571, 3418-3419, 32572-32576, 3421, 32577-32583, 3422, 32584-32596, 3436, 32597-32601, 2845, 32602, 2891, 3441, 3443, 4180, 32603-32620, 3644, 32621-32632, 2682, 32633, 3465, 32634-32641, 30815, 32642-32651, 3475, 32652-32654, 3479, 32655-32693, 3471, 32694, 3733, 32695, 2678, 32696, 18448, 3503, 32697, 2939, 32698-32702, 4179, 32703-32717, 9376, 32718, 3758, 32719-32720, 3525, 32721-32722, 3526, 32723-32732, 3538, 32733-32736, 4197, 32737-32760, 2938, 32761-32768, 3555-3556, 4222, 32769-32770, 3558, 32771-32813, 3366, 32814-32846, 3645, 32847-32865, 3804, 32866-32872, 3611, 32873-32894, 3966, 32895-32898, 3113, 32899, 3628, 32900-32901, 4111, 32902-32919, 3652, 32920-32928, 3642, 32929-32939, 3618, 32940-32953, 2803, 32954-32964, 3658, 32965, 3659, 32966-32986, 3676, 32987-32994, 3679, 32995-32999, 3818, 33000-33006, 1447, 33007-33018, 1045, 33019-33038, 16640, 33039-33041, 3051, 33042-33050, 2795, 33051-33057, 2905, 33058-33063, 3718, 33064-33065, 3896, 33066-33093, 8859, 33094-33100, 5998, 33101-33110, 1527, 33111-33128, 3757, 33129-33141, 3490, 33142-33148, 3849, 33149-33155, 3954, 33156-33161, 4029, 33162-33165, 3591, 33166, 3790, 33167-33174, 2820, 3798, 33175-33194, 3812, 33195-33196, 3816, 33197-33198, 30672, 33199-33210, 2906, 33211-33216, 3834, 33217-33222, 3843, 33223-33252, 3860, 33253-33258, 3863, 33259-33264, 3867, 33265-33271, 3872, 33272-33274, 3892, 33275-33285, 8989, 33286-33292, 5455, 33293-33309, 3914, 33310-33347, 3991, 33348-33349, 3922, 33350, 3923, 33351-33358, 3928, 33359-33360, 3931, 33361, 3932, 33362-33370, 3936, 33371-33372, 2998, 33373-33384, 2662, 3682, 33385-33399, 2714, 33400-33407, 2280, 33408-33409, 3958, 33410-33417, 3963, 33418, 3542, 33419-33427, 3655, 33428-33441, 3972, 33442-33510, 4007, 33511-33531, 4024, 33532-33536, 3130, 33537-33539, 3241, 33540-33566, 4037, 33567-33576, 13, 33577-33578, 4232, 33579-33593, 3096, 33594-33619, 3311, 33620-33640, 4081, 33641-33675, 3183, 33676-33677, 4098, 33678-33681, 4103, 33682-33691, 4308, 33692-33713, 3108, 33714-33716, 3977, 33717-33720, 12914, 33721-33726, 4122, 33727-33728, 2709, 33729-33740, 3731, 33741-33772, 3764, 33773-33797, 3272, 33798-33803, 4297, 33804-33807, 3745, 33808-33810, 4333, 33811-33812, 2913, 4171, 33813, 4173, 33814-33819, 4175, 33820-33822, 4178, 33823-33843, 3429, 33844, 4196, 33845, 3456, 33846-33849, 4202, 33850-33851, 4206, 33852-33873, 3638, 33874-33885, 3672, 33886-33894, 4228, 33895-33898, 4230, 33899-33906, 4335, 33907-33925, 3295, 33926-33928, 2899, 33929-33954, 4267, 33955, 4270, 33956, 3551, 33957-33958, 5575, 33959-33963, 2793, 33964-33966, 4276, 33967-33973, 2750, 33974-33977, 3596, 33978-33992, 4293, 33993-34024, 4189, 34025-34033, 4317, 34034-34041, 3773, 34042-34047, 2874, 34048-34051, 4328, 34052-34055, 4334, 34056, 4336, 34057-34062, 30859, 34063-34068, 3713, 34069-34088, 4589, 34089-34131, 123, 34132-34139, 5041, 34140-34183, 29150, 34184-34191, 4482, 34192-34207, 23580, 34208-34257, 4800, 34258-34322, 4817, 34323, 4636, 34324-34332, 5834, 34333-34373, 5033, 34374-34376, 5903, 34377-34411, 4401, 34412-34452, 14598, 34453-34465, 29749, 34466-34489, 28738, 34490-34497, 4818, 34498-34502, 18566, 34503-34625, 25964, 34626-34664, 4576, 34665-34741, 5906, 34742-34743, 6062, 34744-34745, 33171, 34746-34819, 6120, 34820-34840, 26334, 34841-34847, 26077, 34848-34856, 5226, 34857-34868, 26335, 34869-34875, 4809, 34876-34884, 6148, 34885-34894, 4414, 34895-34913, 6206, 34914-34925, 5313, 34926-34937, 5326, 34938-34969, 5360, 34970-34983, 23677, 34984-35006, 5715, 35007-35032, 4905, 35033-35099, 28976, 35100-35116, 5260, 35117, 4526, 35118-35140, 4533, 35141-35142, 4834, 35143-35227, 5085, 35228-35245, 4868, 35246-35273, 23657, 35274-35313, 12039, 35314-35341, 4773, 35342, 5263, 35343-35384, 4940, 35385-35407, 4929, 35408-35411, 898, 35412-35434, 29905, 35435-35440, 29393, 35441-35462, 4674, 35463-35525, 1151, 35526-35653, 5323, 35654-35677, 5311, 35678-35699, 27025, 35700-35706, 5336, 35707-35721, 5186, 35722-35781, 5825, 35782, 29343, 35783-35832, 32138, 35833-35861, 4980, 35862-35868, 29688, 35869-35897, 4469, 35898-35932, 28581, 35933-35934, 25563, 35935-35961, 4442, 35962-35967, 25816, 35968-35985, 7006, 35986-36006, 6141, 36007-36010, 29803, 36011-36069, 4522, 36070-36120, 29904, 36121-36219, 29774, 36220-36225, 28482, 36226-36250, 5231, 36251, 4629, 36252-36336, 4852, 36337-36346, 4865, 36347-36365, 6157, 36366-36379, 31522, 36380-36421, 4937, 36422-36446, 3596, 36447-36477, 24907, 36478-36495, 5014, 36496-36526, 5067, 36527-36602, 5151, 36603-36666, 4479, 36667-36687, 5236, 36688-36739, 5295, 36740-36766, 4384, 36767-36778, 4883, 36779-36811, 26528, 36812-36820, 5198, 36821-36823, 4363, 36824-36866, 4767, 36867-36924, 30596, 36925-36945, 5238, 36946-36991, 4543, 36992-37020, 33529, 37021-37059, 26648, 37060-37116, 5212, 37117-37140, 5256, 37141-37174, 4741, 37175-37185, 17326, 37186-37215, 23462, 37216-37222, 26965, 37223-37237, 4468, 37238-37279, 5285, 37280-37295, 5245, 37296-37324, 4927, 37325-37358, 963, 37359-37367, 25421, 37368-37391, 5308, 37392-37406, 5015, 37407-37409, 5030, 37410-37424, 24716, 37425-37446, 5280, 37447-37491, 5119, 37492-37496, 30957, 37497-37513, 30267, 37514-37522, 4597, 37523-37526, 5135, 37527-37563, 28220, 37564-37582, 4909, 37583-37601, 27369, 37602-37618, 5258, 37619-37720, 5706, 37721-37722, 5707, 37723-37729, 23462, 37730, 5986, 37731-37733, 5732, 37734-37763, 5756, 37764-37776, 34212, 37777-37781, 5933, 37782-37823, 235, 37824-37829, 5834, 37830-37845, 5871, 37846-37850, 28327, 37851-37886, 27069, 37887-37890, 5754, 37891-37893, 5748, 4818, 37894-37926, 5971, 37927-37935, 5982, 37936-37937, 29582, 37938-37950, 27455, 37951-37970, 6018, 37971-37976, 6090, 37977-37988, 17536, 37989-37991, 28599, 37992-38006, 467, 38007-38027, 26983, 38028-38053, 6120, 38054-38065, 6137, 38066, 6139, 38067-38071, 6174, 38072-38078, 2578, 38079, 6148, 38080-38083, 6012, 38084-38105, 36113, 38106-38111, 16511, 38112-38115, 6195, 38116-38141, 3867, 38142-38148, 5825, 38149-38158, 27598, 38159-38196, 15845, 5894, 38197-38217, 24689, 38218-38261, 5857, 38262-38269, 5863, 38270, 5867, 38271-38319, 6046, 38320-38321, 26402, 38322-38327, 5988, 38328-38337, 5965, 38338-38363, 5850, 38364-38417, 6064, 38418-38428, 5726, 38429-38431, 6082, 38432-38454, 6095, 38455-38461, 23779, 49, 38462-38488, 6144, 38489-38519, 6157, 38520-38524, 6208, 38525-38553, 5866, 38554-38561, 18448, 38562-38585, 24728, 38586-38604, 1263, 38605-38635, 23538, 38636-38639, 5861, 38640-38648, 5854, 38649-38672, 23748, 38673-38681, 1077, 15231, 38682-38685, 25816, 38686-38719, 34352, 38720-38722, 5895, 38723-38726, 15291, 38727-38737, 5751, 38738-38741, 5882, 38742-38750, 4826, 6011, 38751-38755, 5940, 38756-38757, 5943, 38758, 25600, 38759-38784, 5749, 38785-38795, 5952, 38796, 5765, 38797-38818, 6023, 38819-38836, 6126, 38837-38851, 18936, 38852-38853, 5903, 38854-38867, 6037, 38868-38882, 11449, 38883-38916, 36456, 6050, 38917-38936, 36747, 38937-38970, 5784, 38971-38976, 37301, 38977-39000, 5753, 39001-39007, 5755, 39008-39033, 5828, 39034, 7411, 39035-39063, 5818, 5820, 39064-39071, 25358, 39072, 6029, 39073-39076, 25563, 39077-39096, 6004, 39097-39117, 25487, 39118-39121, 5901, 39122-39136, 5798, 39137-39155, 5880, 39156-39174, 5975, 39175-39181, 34189, 39182-39188, 5932, 39189-39194, 26334, 39195-39198, 963, 39199-39200, 5729, 39201-39210, 6014, 39211-39241, 6070, 39242-39246, 5891, 39247-39253, 6206, 39254-39266, 33218, 39267, 19698, 39268-39291, 6145, 39292-39354, 5359, 39355-39363, 6215, 39364-39365, 6218, 39366-39421, 6241, 39422-39434, 7064, 39435-39438, 7639, 39439-39452, 6429, 39453-39467, 6771, 39468, 6335, 39469-39490, 6273, 39491-39523, 6287, 39524, 6288, 39525-39528, 6290, 39529-39534, 7709, 39535-39539, 6493, 39540-39553, 6297, 39554-39556, 6300, 39557-39568, 2957, 39569-39579, 5834, 39580-39581, 6529, 39582-39607, 6329, 39608-39610, 6398, 39611-39619, 5903, 39620-39639, 38621, 39640-39641, 6348, 39642-39654, 6353, 39655-39674, 6362, 39675, 25537, 6367, 39676-39681, 6373, 39682-39683, 6375, 39684-39692, 7488, 39693-39698, 6384, 6311, 39699-39700, 7298, 39701-39705, 6388, 39706-39710, 7283, 39711-39731, 6405, 39732-39758, 23985, 39759, 7592, 39760, 6489, 39761-39764, 37809, 39765-39776, 6848, 6437, 39777-39789, 6445, 39790-39818, 7594, 39819-39843, 6677, 39844-39850, 7122, 39851-39855, 6467, 39856-39868, 6472, 6718, 39869-39888, 6061, 39889-39898, 6671, 39899-39915, 6492, 39916-39924, 7043, 39925-39945, 6094, 39946-39957, 7129, 39958-39981, 6626, 39982-39988, 6531, 39989-40000, 7566, 40001-40014, 7176, 40015, 7197, 40016-40021, 6545, 40022-40035, 7273, 40036-40037, 6554, 6206, 40038-40070, 6882, 40071-40073, 24454, 40074-40078, 6587, 6740, 40079-40085, 7243, 6582, 40086-40091, 6240, 40092-40145, 6624, 40146-40149, 6652, 40150-40166, 6634, 40167-40190, 6640, 40191-40212, 15845, 40213-40237, 7547, 40238-40242, 37906, 40243-40257, 7456, 40258-40272, 7305, 40273-40304, 6697, 40305-40313, 6701, 5835, 40314-40315, 7262, 40316-40358, 6723, 40359-40365, 4691, 40366-40374, 6748, 6728, 6022, 40375-40381, 12039, 40382-40387, 6955, 40388-40410, 6745, 40411-40465, 6225, 40466-40474, 7404, 40475-40494, 6778, 40495-40521, 6809, 40522-40527, 7239, 40528-40531, 6713, 40532-40546, 6802, 40547-40568, 7312, 40569-40582, 6330, 40583-40592, 7236, 40593-40632, 7673, 40633-40639, 6488, 40640-40683, 6863, 40684-40693, 6872, 40694, 6922, 40695-40757, 6900, 40758-40764, 6141, 40765-40767, 7149, 40768-40786, 6465, 40787-40806, 6339, 40807-40819, 261, 40820-40840, 6948, 40841-40849, 6578, 40850, 6953, 40851-40861, 9, 40862-40870, 7458, 40871-40891, 38571, 40892-40905, 6250, 7474, 40906, 6352, 40907-40949, 18502, 40950-40982, 7449, 40983-40999, 7019, 41000-41004, 7023-7024, 41005-41008, 6402, 41009-41024, 7032, 41025-41044, 7038, 41045-41061, 6855, 41062-41068, 6513, 41069-41125, 6678, 41126-41132, 7073, 41133-41156, 6525, 41157-41198, 7195, 41199-41208, 7620, 41209-41215, 7118, 41216, 7120, 41217-41222, 7369, 41223-41243, 7134, 41244-41288, 7158, 41289-41294, 4927, 41295-41311, 6790, 7461, 41312-41331, 7524, 41332-41339, 7185, 41340-41350, 7091, 41351-41357, 7199, 41358-41383, 7222, 41384-41387, 7223, 41388-41398, 7052, 41399-41433, 7240, 41434-41448, 26301, 41449-41454, 7250, 7546, 41455-41461, 6827, 41462-41478, 1625, 41479-41489, 4826, 41490-41492, 7230, 41493-41529, 7274, 41530-41532, 6649, 41533-41546, 7303, 41547-41561, 5198, 41562-41591, 26087, 41592-41596, 7633, 7321, 41597-41607, 24, 41608-41612, 6874, 41613-41619, 6980, 41620-41639, 7342, 41640-41644, 26528, 41645-41647, 7491, 41648, 7349, 41649-41652, 6655, 41653-41665, 6896, 41666-41671, 7360, 41672, 7362, 41673-41691, 7154, 41692-41710, 6884, 41711-41712, 7378, 41713-41735, 7391, 41736-41749, 6310, 7395, 41750-41754, 7536, 7400, 41755-41763, 26077, 41764-41797, 7417, 41798-41836, 6233, 41837-41849, 5825, 7446, 41850-41854, 6425, 41855-41860, 6487, 41861-41883, 7436, 7462, 41884-41912, 7478, 5795, 41913-41933, 7490, 6224, 41934-41937, 7686, 41938, 6686, 41939-41957, 7500, 41958-41975, 6228, 7512, 41976-42008, 6985, 42009-42011, 6486, 42012-42046, 6360, 42047-42067, 7562, 42068-42083, 6800, 42084-42087, 6547, 42088-42089, 6841, 42090-42107, 7582, 42108-42151, 7600, 42152-42159, 6892, 42160-42161, 7469, 42162-42163, 6541, 7608, 42164-42168, 6343, 42169-42206, 7632, 42207-42212, 31144, 42213-42244, 7646, 42245-42247, 25964, 42248-42256, 7653, 42257-42289, 7662, 6646, 7056, 42290-42294, 6942, 42295-42301, 7494, 42302-42307, 5654, 42308-42310, 7678, 42311-42336, 6691, 42337-42369, 7079, 42370-42397, 7734, 42398-42449, 7727, 42450-42457, 8938, 42458-42484, 9508, 7758, 42485-42497, 8293, 42498-42560, 7788, 42561-42565, 8631, 42566-42567, 8309, 42568-42574, 8781, 42575-42594, 8789, 42595-42599, 7724, 42600-42622, 7832, 42623-42647, 8081, 42648-42691, 7852, 42692-42694, 8876, 42695-42750, 235, 42751-42753, 9285, 42754-42763, 8029, 7851, 42764-42794, 7884, 42795-42836, 9515, 42837, 7886, 42838-42849, 32272, 42850-42852, 8677, 42853-42859, 7897, 42860-42872, 7755, 42873-42892, 9260, 42893-42905, 7917, 42906-42913, 7920, 42914-42919, 8891, 42920-42935, 9261, 42936-42943, 7925, 42944-42975, 7933, 42976-42994, 7857, 42995-43049, 7943, 43050-43077, 8411, 43078-43079, 7975, 43080-43088, 7980, 43089-43110, 8772, 43111-43140, 8117, 43141-43145, 37956, 43146-43158, 8005, 43159-43203, 8955, 3596, 43204-43252, 8650, 43253-43272, 8766, 43273-43300, 27903, 43301-43310, 8062, 43311-43326, 8071, 43327-43328, 8571, 43329-43349, 7961, 43350, 41118, 43351-43388, 8096, 43389-43407, 9286, 8102, 43408-43473, 30642, 43474-43475, 8100, 43476-43502, 8401, 43503-43515, 8144, 43516-43517, 8129, 43518-43537, 9312, 43538-43548, 9301, 43549-43558, 29819, 43559-43560, 9178, 43561-43565, 8103, 43566-43573, 9249, 43574-43586, 8206, 605, 43587-43627, 7854, 43628-43667, 8189, 43668-43671, 8815, 43672-43678, 9528, 43679-43680, 8197, 43681-43696, 8263, 43697-43699, 8582, 43700-43706, 9300, 43707-43716, 7822, 43717-43727, 8460, 43728-43761, 8235-8236, 43762-43768, 8240, 43769-43771, 9092, 43772-43780, 8909, 43781-43784, 8763, 43785-43787, 8253, 43788-43789, 9453, 43790-43795, 9441, 43796, 9492, 43797-43805, 8119, 43806-43811, 7812, 43812-43823, 7831, 43824-43854, 9220, 43855-43870, 40744, 43871-43873, 7876, 43874, 8623, 8279, 43875-43902, 9283, 43903, 8989, 43904-43956, 9354, 43957-43986, 8454, 43987-44027, 9200, 44028-44042, 8335, 44043-44046, 8338, 44047-44053, 7840, 44054-44055, 8362, 44056-44079, 8351, 44080-44085, 8356, 44086-44101, 8364, 44102-44109, 29195, 44110-44120, 9329, 44121-44132, 8379, 44133-44162, 8073, 44163-44190, 9399, 44191-44195, 8444, 44196-44198, 8315, 44199-44245, 8506, 44246-44275, 8317, 44276-44313, 8450, 44314-44318, 8447, 45, 44319-44329, 8349, 44330-44369, 7990, 44370-44379, 8459, 44380-44386, 8990, 44387-44463, 9486, 44464-44469, 7945, 44470-44513, 8505, 44514-44515, 9355, 44516-44525, 8620, 44526-44540, 8511, 44541-44542, 8566, 44543-44574, 16511, 8527, 44575-44595, 8539, 44596-44639, 9421, 44640-44648, 8500, 44649-44730, 9142, 44731-44741, 8139, 44742-44758, 8593, 44759-44787, 8008, 44788-44807, 7989, 44808-44880, 8648, 44881-44902, 8546, 44903-44917, 9377, 44918-44968, 9511, 44969-44974, 8540, 44975-44982, 35998, 44983-45021, 8682, 45022-45038, 8396, 45039, 8690, 45040-45061, 9448, 8110, 45062-45086, 8708, 45087-45089, 8893, 8710, 45090-45106, 7983, 45107-45110, 8722, 45111-45166, 8748, 45167-45183, 36163, 45184-45190, 8926, 45191-45211, 9417, 45212-45235, 8767, 45236-45254, 8735, 45255-45290, 8787, 8784, 45291-45305, 29461, 45306-45318, 9325, 45319-45359, 8905, 45360-45365, 8808, 45366-45370, 7951, 45371-45372, 8994, 45373-45383, 7994, 45384-45386, 8461, 45387-45405, 8820, 45406-45413, 7775, 45414-45432, 8825, 45433-45458, 8224, 45459-45472, 9158, 45473-45488, 8714, 45489-45507, 8851, 45508-45516, 8853, 45517-45530, 8859, 45531-45537, 8863, 45538-45559, 7888, 45560-45578, 8931, 45579-45594, 8393, 45595-45597, 8887, 45598-45601, 9425, 45602-45611, 8895, 45612-45687, 8980, 45688-45712, 8922, 45713, 8924, 45714, 9474, 45715-45720, 7806, 45721-45722, 8927, 45723-45733, 8683, 45734, 7955, 45735-45737, 9435, 45738-45758, 8942, 45759, 8006, 45760-45774, 9449, 45775-45779, 8812, 45780-45789, 8080, 45790-45791, 9210, 45792-45806, 8965, 45807-45817, 7881, 45818-45820, 8970, 45821-45827, 8916, 45828-45830, 9442, 45831-45841, 9122, 45842-45844, 8978, 45845-45892, 5455, 45893-45938, 8166, 45939-45942, 9013, 45943-45945, 7960, 45946-45969, 8480, 45970-45979, 9023, 45980, 7970, 45981-45992, 9031, 45993-46004, 7872, 46005-46007, 8504, 46008-46098, 8175, 46099-46104, 9073, 46105-46111, 9079, 46112-46135, 9091, 46136-46144, 8850, 46145-46161, 8797, 46162-46165, 8889, 46166-46171, 8383, 46172-46173, 9353, 46174-46194, 9124, 46195-46197, 8414, 46198-46202, 9131, 46203-46206, 9257, 46207-46217, 8159, 46218-46236, 9139, 46237-46248, 9144, 7896, 46249-46256, 9506, 46257-46270, 9149, 46271-46272, 9159, 46273-46283, 8515, 46284-46287, 9150, 46288-46292, 9160, 46293-46308, 9321, 46309-46321, 8274, 7918, 46322-46329, 8964, 46330-46333, 9184, 46334-46351, 9195, 46352-46354, 9168, 46355-46381, 9471, 46382-46385, 8868, 46386-46455, 9229, 46456-46494, 8711, 46495, 9246, 46496, 8136, 46497-46507, 7923, 46508-46509, 8871, 46510-46519, 9255, 46520-46550, 9268, 46551-46568, 9279, 46569-46572, 9282, 46573-46578, 8387, 46579-46597, 9256, 46598-46600, 8680, 46601-46651, 9306, 46652-46655, 9309, 46656-46658, 9310, 46659-46679, 8280, 46680-46759, 9339, 46760-46788, 9185, 46789, 8570, 46790-46836, 8145, 46837-46851, 8458, 9371, 46852-46860, 9376, 46861-46876, 8959, 46877-46916, 9523, 46917, 9405, 46918-46928, 9527, 46929-46938, 9316, 46939-46948, 9419, 46949-46970, 9430, 46971-46982, 8564, 46983-47006, 9444, 47007-47018, 8846, 47019-47041, 8474, 47042-47051, 9464, 47052-47061, 9469, 47062-47103, 9217, 8348, 47104-47117, 9071, 47118-47167, 8756, 47168-47199, 9516, 47200-47214, 9520, 47215-47222, 8665, 47223-47236, 16044, 47237-47243, 11721, 47244-47249, 14799, 47250-47273, 17040, 47274-47276, 13623, 47277-47278, 16378, 14258, 47279-47284, 14778, 47285-47286, 16921, 47287-47289, 16053, 47290-47294, 13122, 47295, 12011, 47296-47306, 39432, 47307, 15854, 47308-47312, 5748, 47313-47317, 14388, 47318, 13181, 47319-47321, 13138, 47322-47325, 14695, 47326-47328, 14475, 47329-47332, 15347, 11731, 47333-47335, 14478, 47336-47348, 14483, 47349-47359, 14487, 47360-47367, 15828, 47368, 11746, 47369-47377, 14841, 47378-47381, 15420, 47382-47395, 16246, 47396-47401, 13165, 47402, 14503, 47403-47419, 11766, 47420-47424, 39541, 47425-47431, 16994, 47432-47437, 14758, 47438-47441, 13345, 47442, 14528, 47443-47444, 16385, 16116, 47445-47458, 11779, 47459-47460, 15348, 47461-47462, 12874, 235, 14541, 47463-47465, 14504, 47466-47469, 13196, 47470, 11788, 47471-47478, 13804, 47479-47503, 16069, 47504-47509, 14537, 47510-47512, 13293, 47513-47526, 14367, 12832, 47527-47529, 16888, 47530-47531, 13180, 47532-47536, 13218, 47537-47538, 13220, 47539-47566, 14440, 47567, 14587, 47568, 11829, 47569-47576, 31593, 47577-47585, 15960, 47586-47588, 14595, 47589-47592, 14598, 16287, 12539, 47593-47599, 11859, 47600-47603, 16082, 47604-47613, 16559, 47614-47620, 16129, 15849, 47621-47623, 14938, 47624-47626, 12639, 11858, 47627-47630, 13267, 47631, 15982, 47632, 14691, 15605, 13749, 47633-47638, 13213, 47639-47640, 12680, 47641-47649, 14540, 47650-47655, 14627, 47656-47660, 15548, 47661-47672, 12756, 47673-47685, 12910, 47686-47687, 11883, 11906, 47688-47691, 15999, 47692-47710, 11890, 47711, 16822, 47712-47718, 11893, 14907, 47719-47727, 15844, 13128, 47728-47729, 14622, 47730-47734, 13335, 47735-47738, 16026, 47739, 12490, 47740-47746, 15622, 47747-47754, 15579, 47755-47756, 14662, 47757-47759, 12130, 47760-47770, 15813, 47771-47779, 15794, 47780-47783, 11914, 47784-47791, 2216, 47792-47799, 11917-11918, 14677, 47800-47818, 14685, 11728, 47819-47832, 16707, 2493, 47833-47836, 14862, 47837-47841, 23, 47842-47856, 15045, 47857-47859, 14701, 47860-47866, 467, 47867-47869, 11943, 47870-47879, 14687, 47880-47895, 13953, 47896-47899, 13357, 47900-47901, 11959, 47902-47908, 11980, 47909-47911, 15936, 47912, 16968, 47913-47914, 15694, 47915-47919, 14944, 47920-47935, 11976, 14739, 47936-47950, 14744, 47951-47961, 11988, 16089, 47962, 13375, 47963-47978, 11989, 47979-47985, 15812, 47986-47988, 11993, 47989-48001, 15515, 48002-48003, 16235, 12000, 48004-48007, 15986, 48008-48011, 16103, 48012-48014, 12005, 13389, 48015, 28987, 48016-48017, 2564, 48018-48046, 16113, 48047-48052, 16117, 48053-48054, 14263, 48055-48065, 11775, 48066-48074, 12643, 13651, 13921, 16532, 48075-48088, 12027, 48089-48091, 16130, 48092-48109, 15674, 48110-48121, 13762, 48122-48131, 14807, 48132-48143, 14424, 48144-48148, 12135, 48149-48156, 13108, 48157, 14129, 48158-48159, 13019, 48160-48189, 15852, 48190, 13871, 48191-48194, 14826, 48195-48196, 16375, 48197-48225, 13466, 48226-48251, 13474, 48252-48264, 12069, 48265, 13477, 48266-48273, 14851, 48274-48279, 16185, 48280-48286, 42102, 48287-48301, 12086, 13491, 48302-48313, 13495, 48314-48330, 14867, 12097, 48331-48346, 16108, 13166, 48347-48354, 14217, 48355-48363, 13520, 48364-48369, 16184, 48370-48373, 12943, 48374-48406, 13531, 48407-48414, 14896, 48415-48419, 16231, 48420-48438, 13543, 48439-48442, 12139, 15906, 48443-48451, 16171, 48452-48460, 14910, 48461-48471, 14416, 48472-48486, 16252, 48487-48499, 13562, 48500, 13563, 48501, 14923, 12159, 48502, 17090, 48503-48509, 11848, 48510-48514, 14927, 48515, 13401, 48516-48517, 13245, 48518-48521, 38776, 48522-48524, 12580, 48525-48527, 13263, 48528-48532, 14930, 12167, 13583, 48533-48534, 15646, 48535-48551, 13858, 30431, 48552-48556, 14406, 48557-48560, 11868, 48561-48568, 2402, 48569-48572, 15873, 48573-48579, 16625, 48580-48587, 16249, 48588-48592, 14873, 48593-48600, 13995, 48601-48610, 14623, 48611-48616, 13346, 48617-48622, 14310, 48623-48631, 16298, 13615, 48632-48650, 13622, 48651-48660, 16896, 12739, 14531, 48661, 12098, 48662, 13083, 48663-48670, 12215, 48671-48672, 2437, 48673-48674, 16637, 48675-48680, 16771, 48681-48684, 12350, 48685-48698, 15006, 13646, 48699-48707, 15012, 48708-48709, 16308, 12227, 14456, 48710-48728, 13828, 48729, 14802, 48730-48738, 13661, 48739-48744, 14705, 48745-48746, 13286, 48747-48749, 16346, 48750-48759, 16580, 48760, 15709, 48761-48766, 14132, 48767-48772, 12339, 48773-48775, 16662, 48776-48795, 15040, 48796, 14544, 48797-48799, 13820, 48800, 15042, 48801-48803, 12262, 48804-48806, 12702, 48807-48824, 11805, 48825-48827, 13695, 48828-48834, 12276, 48835, 13699, 48836-48841, 12741, 48842-48843, 16503, 48844-48852, 13993, 48853-48859, 13709, 48860-48862, 15882, 48863-48878, 10479, 48879-48899, 12640, 48900-48905, 12305, 48906-48909, 14101, 48910-48923, 12311, 48924-48930, 16702, 48931, 11840, 48932, 12315, 48933-48938, 11911, 48939-48942, 15119, 48943, 13735, 48944-48952, 12770, 48953-48965, 13430, 48966-48982, 13746, 48983-48987, 12050, 48988-48998, 12025, 48999-49008, 15431, 49009-49011, 2580, 49012-49016, 12336, 49017-49025, 16435, 15112, 49026-49042, 17034, 49043-49046, 14580, 49047-49057, 15120, 12351, 49058-49059, 14659, 49060-49063, 16866, 49064-49070, 16459, 49071-49082, 15131, 49083-49094, 10589, 49095-49097, 15134, 49098-49110, 15317, 49111-49123, 15459, 49124-49131, 15869, 49132-49161, 12390, 49162-49168, 14218, 49169, 15154, 49170-49172, 2285, 49173-49175, 14746, 49176, 16796, 49177-49184, 13507, 49185-49196, 2451, 49197-49210, 14113, 16884, 49211-49216, 16498, 49217-49219, 16499, 49220-49223, 13691, 49224-49228, 12418, 49229-49251, 11810, 49252-49259, 15183, 49260-49261, 16511, 49262-49263, 16512, 49264-49265, 16607, 49266, 15190, 49267-49269, 16074, 49270-49276, 15494, 49277-49278, 13148, 49279-49281, 15196, 49282-49284, 13846, 49285, 15954, 49286, 3867, 12839, 49287-49292, 12194, 49293-49304, 15176, 16527, 49305-49314, 13855, 49315-49318, 16061, 49319-49325, 15211, 49326-49327, 13857, 16784, 49328-49361, 15153, 49362, 13265, 14208, 49363-49370, 13868, 49371-49373, 15225, 49374, 13276, 49375-49377, 12996, 49378-49387, 13874, 49388-49392, 15231, 49393-49404, 15796, 49405-49413, 13008, 49414-49415, 12485, 49416-49420, 12324, 49421-49434, 16163, 49435-49457, 16583, 49458, 12947, 49459-49463, 16586, 49464-49475, 16310, 49476-49480, 13003, 1399, 49481-49484, 13907, 49485-49489, 16600, 49490-49500, 12509, 49501-49503, 13944, 49504, 15271, 36268, 49505-49509, 15274, 49510-49514, 15541, 49515-49519, 15841, 49520-49531, 15278, 49532-49535, 12508, 49536-49544, 12529, 49545-49547, 16620, 49548-49560, 16626, 49561-49565, 16629, 49566-49574, 15291, 49575-49577, 16635, 49578-49588, 13956, 49589-49597, 16520, 49598-49602, 15301, 49603-49605, 13962, 49606-49616, 13933, 49617-49630, 16216, 49631-49642, 14893, 49643, 16655, 49644-49646, 16079, 49647-49656, 13976, 49657-49679, 15627, 49680-49690, 15259, 49691-49696, 13269, 13986, 49697-49700, 16479, 49701-49703, 15321, 49704-49711, 15010, 17094, 49712-49714, 13809, 49715-49719, 13382, 49720-49731, 12611, 15076, 49732-49733, 12604, 49734-49749, 12803, 49750, 12616, 49751-49754, 12431, 49755-49757, 16690, 49758-49764, 15727, 49765-49770, 14011, 49771-49773, 12790, 49774-49780, 12623, 49781-49786, 16146, 49787, 14018, 49788-49800, 13, 49801-49821, 13062, 49822-49831, 11712, 49832-49836, 16713, 49837-49839, 15365, 49840-49846, 13436, 49847-49848, 12654, 49849-49851, 16439, 15299, 49852-49871, 15671, 49872-49875, 16724, 49876-49880, 12668, 49881, 12823, 49882-49888, 13936, 49889-49895, 12675, 49896-49909, 11862, 49910-49916, 12822, 49917-49918, 13947, 49919-49934, 15404, 14149, 14070, 49935-49938, 12160, 49939-49941, 13222, 49942-49947, 29048, 49948-49951, 12121, 49952-49953, 15929, 49954-49955, 15413, 49956-49957, 16756, 49958-49961, 15417, 49962-49963, 13899, 49964-49973, 15908, 49974, 12178, 49975-49977, 17067, 49978-49979, 17062, 49980, 14963, 49981-49983, 12721, 49984, 12494, 49985-49987, 12767, 49988-49989, 12541, 49990-49992, 12619, 49993-49994, 14865, 49995-49998, 15428, 49999-50006, 16782, 50007-50011, 14087, 50012-50016, 14097, 50017, 6492, 50018-50022, 16941, 12736, 50023-50024, 16091, 12740, 50025-50033, 12582, 50034-50047, 15443, 50048-50057, 14107, 50058-50068, 12092, 50069, 16328, 50070, 14866, 50071-50074, 15179, 15458, 50075-50083, 36845, 50084-50126, 15836, 50127-50141, 14065, 50142-50173, 2297, 50174-50179, 14147, 50180-50190, 14143, 50191-50193, 12799, 50194-50198, 12800, 50199-50209, 14151, 50210-50225, 15516, 13499, 50226-50238, 12811, 12357, 13515, 50239-50265, 15555, 50266-50271, 14872, 50272-50273, 15525, 50274-50275, 15526, 50276-50285, 12827, 50286-50290, 16870, 50291-50292, 12600, 50293-50297, 14192, 50298, 15535, 50299-50306, 12837, 50307-50315, 12841, 50316-50318, 12111, 50319-50349, 15610, 50350-50354, 16011, 50355-50362, 13056, 50363-50364, 2366, 50365-50384, 12860, 50385-50387, 2372, 50388-50393, 12865, 50394-50413, 16473, 50414-50415, 12877, 50416, 4741, 50417-50434, 15583, 50435-50436, 14434, 50437, 14257, 50438, 12290, 50439-50444, 15587, 12889, 12233, 50445-50473, 16915, 50474-50475, 16918, 50476-50482, 14249, 50483-50486, 15862, 12685, 50487-50498, 15608, 50499-50522, 16928, 12914, 50523-50526, 13952, 50527-50553, 12928, 50554-50563, 16947, 50564-50566, 16949, 50567, 13074, 50568-50576, 12127, 50577-50580, 15645, 50581-50594, 16962, 50595, 15649, 50596-50599, 15655, 50600-50606, 16966, 50607, 17072, 50608-50609, 12424, 50610-50616, 13739, 50617-50618, 12132, 50619-50624, 14297, 50625-50627, 12953, 50628-50634, 13141, 50635-50642, 12481, 50643-50648, 13203, 50649-50655, 16981, 50656-50664, 16644, 50665-50668, 14817, 50669-50677, 2559, 50678-50681, 15410, 16643, 50682-50691, 16488, 50692-50694, 12976, 50695-50704, 2498, 50705-50709, 12986, 50710-50732, 15707, 50733-50745, 1406, 50746-50749, 14029, 50750-50755, 15717, 50756-50757, 13761, 50758-50759, 14336, 50760-50779, 16028, 50780-50782, 14903, 50783-50788, 15345, 50789-50810, 13631, 50811-50813, 32687, 50814-50821, 12562, 50822, 15747, 50823-50826, 15549, 50827-50830, 16027, 50831-50835, 13581, 50836-50843, 12205, 50844-50850, 15372, 50851-50865, 14153, 50866, 14955, 50867-50897, 13089, 50898-50904, 14387, 50905-50911, 17064, 50912, 17065, 14394, 50913-50915, 13510, 14934, 50916-50922, 15788, 50923-50924, 15467, 50925-50934, 15682, 50935-50940, 17076, 50941-50967, 12960, 50968, 13092, 50969-50970, 13588, 50971-50982, 15821, 50983-50987, 13737, 50988-50995, 19321, 50996-51003, 18229, 51004-51007, 18288, 51008-51012, 19437, 17110, 17726, 51013-51025, 19704, 51026-51029, 17124, 51030-51041, 17130, 51042-51090, 23, 18783, 19686, 51091-51094, 19363, 51095, 18621, 19318, 51096-51107, 18443, 51108-51109, 17167, 51110-51121, 17173, 51122-51132, 17177, 51133-51148, 17185, 51149-51151, 17143, 51152-51160, 18324, 51161-51163, 18054, 51164-51169, 18936, 51170-51175, 17198, 51176-51189, 18354, 51190, 17775, 51191-51198, 17214, 51199-51213, 17221, 51214-51221, 18923, 51222-51230, 18315, 51231-51237, 17398, 17238, 51238-51241, 17242, 51242-51248, 18059, 51249-51258, 19763, 51259-51266, 17256, 51267-51273, 2737, 51274-51279, 18470, 51280-51325, 18376, 51326-51345, 18449, 51346-51383, 17335, 51384-51408, 18040, 51409-51415, 17858, 51416-51421, 17360, 51422-51449, 17197, 51450-51461, 17447, 51462-51463, 17382, 51464-51465, 17384-17385, 18962, 51466-51470, 19432, 51471-51480, 18566, 51481-51506, 19793, 51507-51524, 17419, 51525-51526, 17422, 51527-51573, 17351, 51574-51581, 19424, 51582-51597, 18047, 51598-51617, 17391, 51618-51652, 17945, 51653-51678, 17594, 51679-51691, 19693, 19405, 51692-51723, 19176, 51724, 17533, 51725-51726, 17535, 51727-51744, 17542, 51745-51746, 17607, 51747-51749, 17502, 51750-51760, 467, 51761-51774, 17564, 51775-51789, 17571, 51790-51792, 17596, 51793-51804, 17583, 51805-51816, 17749, 51817-51851, 17602, 51852-51864, 18159, 51865-51867, 2947, 51868-51889, 18392, 51890-51891, 17628, 51892-51896, 17152, 51897-51921, 18450, 51922, 19794, 51923-51930, 18920, 51931-51935, 17693, 51936-51958, 2993, 51959, 2578, 51960-51963, 18900, 51964-51981, 17682, 51982-51990, 17688, 51991-52007, 18175, 52008-52031, 19527, 52032-52038, 18280, 17724, 52039-52055, 19631, 52056-52069, 17742, 52070-52074, 17329, 52075-52092, 17347, 52093-52100, 3162, 17770, 52101-52113, 17780, 52114-52126, 17786, 52127-52129, 17789, 52130-52168, 28939, 17811, 52169-52173, 17817, 52174-52196, 17782, 52197-52214, 19287, 52215-52237, 17275, 52238, 36671, 52239-52258, 17875, 19148, 52259-52264, 17880, 52265, 17556, 29009, 52266-52288, 6287, 52289-52303, 17898, 52304-52311, 17903, 52312-52314, 17272, 52315-52316, 18902, 52317, 17673, 18564, 52318-52321, 19406, 52322-52324, 17912, 52325, 17913, 52326-52331, 32172, 52332-52335, 17920, 52336-52342, 19120, 52343-52360, 5444, 52361-52379, 17663, 52380-52391, 17795, 52392-52401, 19412, 52402-52404, 19554, 52405-52408, 19804, 52409-52413, 37352, 52414-52421, 19397, 52422-52425, 19098, 52426-52443, 19727, 52444-52450, 18011, 52451-52472, 17867, 52473-52479, 18032, 52480-52492, 18038, 52493-52501, 19754, 52502-52508, 18048-18049, 52509-52514, 19375, 52515-52518, 17731, 52519-52525, 19026, 52526-52551, 18507, 52552-52553, 18075, 52554-52561, 19201, 52562-52563, 18082, 52564-52574, 40486, 52575-52579, 38332, 52580-52591, 18094-18095, 52592-52599, 18099, 52600, 17845, 52601-52628, 18397, 52629-52635, 18859, 52636-52647, 19266, 52648-52651, 18144, 52652-52663, 18722, 52664-52689, 18122, 52690-52701, 18169, 52702-52719, 17407, 52720-52743, 18193, 52744-52746, 18967, 52747-52752, 18858, 52753, 18200, 18269, 52754-52758, 18202, 52759-52763, 18793, 52764-52768, 18664, 52769-52772, 18706, 52773, 19520, 52774-52789, 17135, 52790-52791, 17294, 52792-52796, 17610, 52797-52805, 18642, 52806-52814, 18227, 52815-52825, 18237, 52826-52835, 17171, 52836-52842, 18710, 52843-52870, 19104, 52871-52878, 9956, 52879-52922, 18312, 52923-52940, 18322, 52941-52955, 18500, 52956-52959, 18337, 52960-52971, 18352, 52972-52973, 18355, 52974-52983, 18359, 52984-53001, 18696, 53002-53011, 18258, 53012-53017, 17199, 53018-53020, 18383, 53021-53039, 18463, 53040-53065, 19153, 18799, 53066-53079, 7863, 53080-53084, 17711, 53085-53092, 18442, 53093-53101, 18448, 53102-53125, 18458, 53126-53132, 17291, 53133-53136, 17981, 19440, 53137-53145, 19319, 53146-53155, 19134, 53156-53160, 18489, 53161-53186, 18502, 53187-53194, 18509, 53195-53205, 18518, 53206-53207, 17768, 53208-53240, 17686, 53241-53251, 18796, 53252-53253, 17518, 53254-53282, 18559, 53283-53290, 18966, 53291-53297, 19174, 53298-53316, 18930, 53317-53330, 18063, 53331-53337, 19496, 53338-53340, 18234, 53341, 27629, 18595, 53342-53353, 17551, 53354-53431, 18656, 53432-53436, 17929, 53437-53440, 17464, 53441-53442, 18707, 53443-53451, 17450, 53452-53476, 18908, 53477-53493, 18699, 53494-53511, 18838, 53512-53518, 18713, 53519-53532, 18514, 53533-53540, 18730, 53541, 18731-18732, 53542-53551, 19585, 53552-53562, 16439, 53563-53568, 18754, 4826, 53569-53578, 19377, 53579-53584, 18763, 53585-53612, 16640, 53613-53632, 18794, 19487, 53633-53668, 19506, 53669-53677, 19444, 53678-53691, 18833, 53692-53693, 17754, 53694-53700, 19038, 53701-53713, 19200, 53714-53747, 19339, 53748-53757, 33911, 18877, 53758-53771, 17344, 53772-53789, 17986, 53790-53812, 18527, 17639, 53813-53816, 31144, 18268, 53817-53818, 18922, 53819-53832, 18934, 53833, 18951, 53834-53889, 18423, 53890-53893, 18281, 53894-53904, 19624, 53905-53925, 2300, 53926-53929, 18903, 53930-53935, 17964, 53936, 18995, 53937-53952, 17735, 53953-53979, 19024, 53980-53987, 19030, 53988-54000, 18761, 54001-54024, 19054, 54025-54052, 19070, 54053-54056, 19073, 54057-54074, 19086, 18556, 54075-54104, 19109, 54105, 19110, 54106-54110, 19113, 54111, 19115, 54112-54125, 17872, 18739, 54126-54163, 17290, 54164, 19150, 54165-54167, 19152, 54168-54176, 19156, 54177-54181, 18780, 54182-54198, 19569, 54199-54204, 18561, 54205-54208, 19182, 19184, 54209-54229, 19204, 54230-54235, 19301, 54236-54246, 18918, 54247-54250, 17431, 54251-54253, 17897, 54254-54265, 19618, 54266, 19445, 54267-54269, 17904, 54270-54280, 19237, 54281, 19027, 54282-54288, 19243, 54289-54307, 7411, 54308-54342, 19283, 54343-54355, 17349, 54356-54360, 19428, 54361-54386, 19636, 54387-54392, 19317, 54393-54410, 18909, 54411-54421, 19813, 54422-54431, 18515, 54432-54466, 18476, 54467-54469, 4061, 54470-54473, 19719, 54474-54492, 19381, 54493-54501, 19391, 54502-54508, 19400, 54509-54528, 19755, 54529-54530, 17463, 54531-54548, 19127, 54549-54614, 19460, 54615-54634, 19475, 54635-54640, 19479, 54641-54646, 19484, 54647-54666, 12341, 54667-54675, 19504, 54676-54681, 17662, 54682-54683, 19509, 54684-54685, 19512, 54686-54693, 19492, 54694-54707, 19523, 54708-54710, 19526, 17778, 54711-54717, 18749, 54718-54741, 19335, 18070, 54742-54744, 18079, 54745-54760, 19562, 54761-54806, 19587, 54807-54825, 19760, 19388, 54826-54838, 19610, 54839-54846, 19354, 54847-54871, 12985, 54872-54895, 18715, 54896-54917, 17252, 54918-54931, 19395, 54932-54939, 19571, 54940-54958, 18424, 54959-54973, 19698, 54974-54976, 19700, 54977-54978, 19702, 54979-54996, 19711, 54997-55000, 18205, 55001-55009, 18875, 55010-55018, 17203, 55019-55074, 19756, 55075-55081, 3128, 55082-55085, 19463, 55086-55096, 19773, 55097-55116, 19577, 55117-55124, 19790, 55125-55129, 17750, 55130-55150, 19809, 55151-55183, 17983, 55184-55202, 21608, 55203-55227, 19876, 55228-55267, 20385, 55268-55273, 19998, 55274-55290, 19973, 55291-55306, 20002, 55307-55326, 20032, 20131, 55327, 898, 55328-55329, 21251, 55330-55343, 22844, 55344-55348, 20087, 55349-55362, 21540, 55363-55369, 20130, 55370-55380, 22680, 55381-55382, 22758, 55383-55389, 20171, 55390-55412, 21963, 55413-55414, 21103, 55415-55416, 21752, 55417-55435, 20260, 55436-55438, 20025, 55439-55450, 20280, 55451, 25964, 55452-55466, 20304, 55467-55505, 467, 55506-55526, 22923, 55527-55571, 20500, 55572-55574, 20503, 55575-55579, 20516, 55580-55584, 20521, 55585, 20528, 55586-55589, 20540, 55590-55594, 20558, 55595-55621, 19937, 55622-55629, 21805, 55630-55633, 21355, 55634-55636, 2082, 55637-55654, 20058, 55655-55685, 20695, 55686-55689, 20701, 55690-55715, 19962, 55716-55720, 20765, 55721-55731, 22058, 55732-55745, 22770, 55746-55753, 21179, 55754-55756, 41, 55757-55765, 20823, 55766, 21988, 55767, 20997, 22474, 55768-55779, 20848, 55780-55784, 20697, 55785-55787, 20861, 55788-55789, 20337, 55790-55816, 20918, 55817-55823, 12174, 55824-55825, 20931, 55826-55834, 20310, 55835-55864, 21664, 55865-55882, 22465, 55883-55885, 21033, 55886-55888, 21037, 55889-55890, 19847, 55891-55895, 21053, 55896-55913, 21070, 55914, 21086, 55915-55916, 21093, 55917-55931, 21117, 55932-55942, 22364, 55943-55958, 21765, 55959-55987, 21194, 55988-55996, 20355, 55997-56002, 21220, 56003-56016, 21241, 56017-56034, 22280, 56035, 21284, 56036-56045, 25360, 56046-56057, 20060, 56058-56063, 21671, 56064-56098, 21399, 22888, 56099-56119, 22809, 56120-56146, 21483, 56147-56168, 20096, 56169-56173, 21889, 56174, 21537, 21262, 56175-56196, 21579, 56197-56216, 21063, 21327, 56217-56262, 20499, 56263-56281, 21923, 21459, 56282-56297, 5906, 56298, 19890, 56299-56304, 21754, 56305-56321, 21776, 56322-56331, 20727, 21749, 21790, 56332-56354, 21837, 56355-56361, 21848, 56362-56379, 21871, 56380-56389, 5575, 56390-56406, 21924, 56407-56440, 20350, 56441, 22826, 20386, 56442-56445, 22295, 56446-56449, 21987, 56450, 22137, 56451-56454, 20312, 56455-56462, 22013, 56463, 22018, 56464-56466, 22025, 56467-56468, 22037, 56469, 21681, 56470-56486, 22549, 21730, 56487-56489, 22076, 56490-56500, 21157, 56501-56514, 22121, 56515-56527, 19945, 56528-56533, 19883, 56534-56542, 21193, 56543-56558, 22203, 56559, 20537, 56560-56583, 22235, 56584-56596, 21791, 56597-56600, 22263, 56601-56625, 21083, 56626-56636, 20766, 56637-56638, 22325, 56639-56650, 22345, 56651-56661, 22373, 56662-56665, 25358, 56666-56690, 22412, 56691-56720, 22459, 56721-56728, 21478, 56729-56748, 21242, 56749-56758, 21539, 56759-56765, 22570, 56766-56781, 21391, 56782-56785, 22605, 56786, 22606, 56787-56797, 22623, 56798-56799, 22245, 20670, 56800-56810, 27147, 56811-56814, 21210, 56815, 22657, 56816-56851, 22703, 56852-56870, 22747, 56871-56882, 5150, 56883-56907, 22823, 56908-56911, 19913, 56912-56933, 21801, 56934-56963, 22712, 56964-56966, 21787, 56967-56986, 20031, 56987-56991, 22161, 19868, 56992-56998, 20357, 56999-57016, 20982, 57017-57020, 19920, 57021-57033, 20773, 57034-57041, 19947, 57042-57046, 19952, 57047-57055, 19969, 57056-57057, 22447, 57058-57061, 21853, 57062-57069, 20003, 57070-57078, 20381, 57079-57082, 20036, 57083-57103, 22850, 57104-57111, 5871, 57112-57116, 20527, 57117-57159, 21036, 57160-57172, 20196, 57173-57180, 20919, 57181-57186, 20219, 21576, 57187-57212, 22423, 57213-57240, 20443, 57241-57270, 23, 57271-57274, 24147, 57275-57281, 20013, 57282-57292, 21900, 57293-57319, 20456, 57320-57335, 20479, 57336-57337, 21967, 57338-57342, 20510, 57343-57350, 20368, 57351-57367, 20567, 57368-57428, 20654, 57429-57439, 20671, 57440-57445, 20677, 57446-57470, 20721, 57471-57563, 22270, 57564-57598, 20849, 57599-57627, 20932, 57628-57634, 20941, 57635-57643, 20953, 57644-57704, 20992, 57705, 21068, 57706-57709, 21061, 57710-57716, 21088, 57717-57781, 21196, 57782-57790, 21214, 57791-57793, 21218, 57794-57811, 21255, 57812-57814, 21771, 57815-57839, 20582, 57840-57858, 21331, 57859-57862, 21342, 57863-57890, 21400, 57891-57899, 21441, 57900-57905, 21422, 57906-57913, 19908, 57914-57922, 2285, 57923-57926, 22880, 57927-57929, 21074, 57930-57933, 21974, 57934-57935, 21475, 21479, 57936-57937, 20156, 57938-57944, 21506, 57945-57955, 21691, 57956-57970, 21553, 57971-57974, 21558, 57975-58040, 1371, 58041-58074, 31197, 58075-58103, 20681, 58104-58113, 21655, 58114-58144, 22435, 58145-58151, 21846, 58152, 21850, 58153-58177, 21652, 20630, 58178-58180, 22248, 58181, 21894, 58182-58195, 21911, 58196-58202, 21002, 58203-58225, 22593, 58226-58237, 20429, 58238-58243, 21981, 58244-58254, 22160, 22780, 58255-58259, 20556, 58260-58264, 22036, 58265-58273, 22050, 58274-58313, 21501, 58314-58339, 22159, 58340-58345, 22166, 58346-58357, 20430, 58358, 21480, 58359-58366, 20883, 58367-58373, 22206, 58374-58393, 22222, 22225, 58394-58403, 22249, 20084, 58404-58406, 22256, 58407-58473, 22340, 58474-58489, 22367, 58490-58508, 20378, 58509, 22400, 58510-58544, 21637, 58545-58559, 20807, 58560, 30964, 58561-58574, 30872, 58575-58581, 22598, 58582-58601, 30908, 58602-58611, 21081, 58612, 22561, 58613-58622, 6148, 58623-58637, 20829, 58638-58641, 22595, 58642-58665, 22883, 58666-58679, 2051, 58680-58691, 22679, 58692, 22685, 58693-58695, 22687, 58696, 22907, 58697-58713, 22700, 58714-58754, 22760, 58755-58767, 22771, 58768-58772, 22879, 58773-58786, 20110, 58787-58857, 21426, 58858-58879, 22935, 58880-58888, 22512, 58889-58901, 19864, 58902-58943, 21929, 58944-58969, 19968, 58970-58972, 20822, 58973-58988, 19997, 58989-59003, 22308, 59004-59026, 20080, 59027-59031, 21517, 59032-59051, 20135, 59052-59059, 23214, 59060-59083, 20188, 59084-59101, 31122, 59102-59104, 29741, 59105-59110, 20245, 59111-59140, 20283, 59141-59155, 22719, 59156-59166, 21980, 59167, 28577, 59168-59192, 20360, 59193-59203, 19835, 59204-59219, 27903, 59220-59278, 20485, 59279-59295, 20811, 59296-59299, 20529, 59300-59302, 21899, 59303-59319, 6206, 59320-59332, 605, 22455, 59333-59340, 20607, 59341-59357, 20629, 20632, 59358-59359, 21592, 59360-59386, 20665, 59387-59445, 21432, 59446-59451, 22209, 59452-59470, 21376, 59471-59472, 22060, 59473-59480, 20307, 59481-59520, 22898, 59521-59525, 20875-20876, 59526-59537, 21435, 20897, 59538-59550, 19984, 59551-59556, 20473, 59557-59564, 20926, 59565-59571, 20579, 59572, 20270, 59573-59587, 20961, 59588-59606, 20593, 59607-59629, 21077, 59630-59666, 235, 59667-59668, 19974, 59669-59673, 21121, 59674-59715, 21184, 59716-59732, 20064, 59733-59734, 22546, 21212, 59735-59745, 21237, 59746-59763, 22197, 59764-59783, 22839, 59784-59791, 21311, 59792-59794, 5825, 59795, 20584, 59796-59811, 22288, 59812-59818, 21363, 59819-59821, 21370, 59822-59868, 21430, 59869-59906, 21487, 59907-59950, 25693, 59951-59952, 22344, 59953-59956, 21569, 59957-59967, 20129, 59968-59987, 21624, 59988, 21626, 59989-59991, 19860, 59992-59993, 21743, 20779, 59994-60024, 21703, 60025-60037, 21724, 60038-60049, 20137, 60050-60081, 11104, 60082-60088, 21510, 60089-60093, 22566, 60094-60107, 22237, 60108-60125, 30187, 60126-60147, 22875, 60148-60155, 22908, 60156, 21914, 60157-60172, 19993, 60173-60174, 19885, 60175-60180, 5903, 60181-60187, 21956, 60188-60209, 21975, 60210-60262, 20890, 60263-60315, 22192, 60316-60332, 30530, 60333-60359, 21643, 60360-60386, 22189, 60387-60389, 22306, 60390-60395, 22317, 22571, 60396-60442, 22015, 60443-60446, 21147, 60447-60450, 39081, 60451-60467, 22153, 60468-60510, 22472, 60511-60521, 20268, 60522-60558, 20736, 60559-60573, 21590, 60574-60583, 22085, 60584-60598, 22626, 60599-60629, 27191, 60630-60654, 22764, 60655-60676, 21429, 60677-60703, 21127, 60704, 20476, 60705-60712, 20894, 60713-60729, 22833, 60730-60765, 22741, 60766-60783, 20628, 60784-60830, 19897, 60831, 22236, 60832-60852, 19930, 60853-60854, 19942, 60855-60859, 19950, 60860-60863, 22502, 60864-60870, 6287, 60871-60872, 19976, 60873-60923, 20062, 60924, 21278, 60925-60931, 21414, 60932-60950, 20119, 60951-60969, 20324, 60970-60971, 19848, 60972-60993, 20625, 21838, 60994-61001, 22568, 61002-61018, 20232, 61019-61023, 20943, 61024-61025, 22199, 61026-61049, 21458, 61050-61058, 20289, 61059-61064, 20617, 61065, 20301, 61066-61072, 20322, 61073-61085, 20338, 61086-61092, 20348, 61093-61109, 21488, 61110-61115, 21072, 61116-61126, 21146, 61127-61128, 21286, 61129-61149, 20457, 61150, 20488, 61151-61187, 21983, 61188-61196, 20539, 20542, 61197-61200, 20216, 61201-61205, 20565, 61206-61244, 20624, 61245-61252, 21418, 61253-61260, 20199, 61261-61292, 20679, 61293-61306, 21433, 61307-61318, 20732, 61319-61342, 20769, 61343-61363, 22310, 61364-61368, 20815, 61369-61375, 21710, 61376-61386, 21951, 61387-61393, 21378, 20850, 61394-61397, 22335, 61398-61402, 19834, 61403-61411, 21584, 61412-61418, 12217, 61419-61428, 20908, 61429-61486, 21812, 61487-61491, 21040, 61492-61495, 21044, 61496-61502, 21055, 61503-61535, 21104, 61536-61552, 19850, 61553-61566, 24913, 61567-61581, 20590, 61582-61586, 22939, 61587-61590, 21188, 61591-61642, 21256, 61643-61644, 21260, 61645-61646, 21263, 61647-61651, 21940, 61652-61658, 22331, 61659-61666, 21312, 61667-61673, 21323, 21326, 22723, 61674-61675, 21030, 61676-61678, 21354, 61679-61685, 21719, 61686-61690, 25453, 61691-61704, 21407, 61705-61714, 21582, 61715-61726, 28581, 61727, 20792, 61728-61756, 21493, 61757-61764, 20704, 61765-61775, 21543, 61776-61778, 21300, 61779-61794, 21198, 61795-61799, 21598, 61800-61806, 21609, 61807-61815, 21708, 61816-61850, 20011, 61851-61880, 22731, 61881-61899, 31269, 61900-61906, 22139, 61907-61915, 4826, 61916-61934, 20070, 61935-61936, 21802, 61937-61947, 22362, 61948-61990, 22729, 22736, 61991-62002, 22202, 62003-62008, 20206, 62009-62016, 21925, 62017-62022, 22077, 62023-62045, 21046, 62046-62069, 22016, 62070-62083, 3867, 62084-62091, 22059, 62092-62104, 22091, 62105-62114, 21949, 62115-62127, 22629, 22131, 62128, 22135, 62129-62145, 22165, 62146-62199, 22234, 62200-62201, 22240, 62202-62206, 21920, 62207-62210, 21470, 62211-62237, 30630, 62238-62250, 7411, 62251-62264, 22332, 62265-62266, 22333, 62267-62270, 19852, 62271-62320, 22940, 62321, 22755, 62322-62361, 21810, 62362-62366, 22514, 62367-62380, 22864, 62381-62398, 22558, 62399-62404, 20789, 62405-62418, 22283, 62419-62430, 22600, 62431-62435, 21078, 62436-62449, 22634, 62450-62461, 22132, 22652, 62462-62464, 20937, 62465-62472, 22675, 62473-62540, 22767, 62541-62554, 31178, 62555-62583, 20201, 62584-62598, 22861, 62599-62615, 20042, 62616-62642, 21685, 62643-62645, 26528, 62646-62653, 22948, 62654-62664, 22956, 5903, 62665-62669, 23243, 62670-62698, 21086, 62699, 23657, 62700, 2280, 62701-62703, 60815, 62704, 21802, 62705-62708, 24955, 62709-62712, 22989, 62713-62726, 23410, 62727-62757, 23394, 62758-62760, 23173, 23230, 62761-62803, 21540, 62804-62805, 5506, 21159, 7411, 62806, 23384, 62807, 22997, 62808-62814, 56211, 62815-62820, 24689, 62821-62829, 58973, 62830-62853, 58987, 62854-62863, 23058, 62864, 23060, 62865-62866, 23282, 62867, 23062, 62868, 5825, 62869-62891, 24196, 20350, 62892, 20359, 62893-62898, 5834, 23200, 62899-62900, 23201, 62901, 23405, 23393, 62902-62906, 23202, 62907-62910, 23248, 62911-62915, 23090, 62916-62924, 23095, 62925-62935, 21673, 62936-62946, 23215, 62947-62957, 23316, 62958-62960, 23292, 62961-62971, 23074, 62972-62978, 23129, 62979-62991, 59559, 62992-62994, 23002, 62995-62996, 23144, 62997-63000, 60968, 63001, 24913, 63002-63003, 23371, 63004-63012, 25581, 63013-63021, 23266, 23163, 63022-63024, 26140, 63025-63026, 62366, 63027-63030, 23169, 20961, 63031-63034, 23937, 63035-63041, 4826, 63042-63043, 37, 63044-63050, 23180, 63051-63052, 23378, 63053-63056, 23184, 38513, 58113, 63057-63060, 22502, 63061, 23191, 63062-63064, 5527, 63065-63071, 25004, 63072-63079, 20232, 23207, 23407, 63080-63085, 23211, 63086-63107, 23007, 63108-63111, 22634, 63112-63124, 57619, 63125, 23895, 63126, 58350, 23232, 63127-63128, 26939, 63129, 23322, 23052, 63130-63139, 23098, 63140-63142, 23245, 63143-63155, 23190, 63156-63157, 59157, 63158-63171, 21884, 63172-63173, 22977, 63174-63178, 5575, 63179-63190, 27191, 63191-63192, 23148, 63193-63194, 23154, 63195-63198, 23164, 20346, 63199, 23283, 23324, 63200-63202, 23175, 63203-63210, 23310, 63211-63224, 22962, 63225-63230, 23225, 63231-63240, 13, 63241-63242, 60195, 63243-63253, 24774, 63254-63262, 23307, 63263-63272, 23314, 63273-63276, 23252, 63277-63285, 23132, 63286-63287, 23325, 63288-63289, 23359, 59242, 63290-63301, 23331, 63302-63307, 23369, 63308-63314, 62307, 63315-63319, 31178, 23351, 61330, 25964, 63320-63330, 23357, 63331-63342, 22973, 63343-63344, 23362, 63345, 23363, 23174, 63346-63350, 23372, 63351, 23373, 23271, 63352, 23374, 63353-63354, 23317, 63355, 34329, 6145, 63356-63367, 27369, 63368-63373, 23383, 23188, 63374-63378, 23249, 63379-63381, 3983, 63382-63385, 23273, 63386-63408, 6206, 63409-63413, 23023, 63414-63429, 23401, 63430, 23402, 23404, 63431-63433, 23152, 63434-63435, 12039, 63436-63438, 23048, 63439-63441, 61632, 23416, 63442-63444, 15845, 63445-63446, 23171, 63447-63448, 3305, 63449-63476, 23450-23451, 63477-63485, 23491, 63486-63490, 23506, 27145, 63491-63494, 23519, 36872, 63495-63515, 26011, 63516-63528, 23456, 63529-63541, 23665, 63542-63543, 23675, 63544-63555, 23133, 63556-63565, 17281, 63566-63586, 27088, 24996, 63587-63598, 23854, 63599-63602, 23876, 11852, 63603-63611, 23895, 63612-63614, 23903, 63615-63619, 23923, 63620-63631, 23985, 63632-63650, 24034, 27126, 63651-63658, 24079, 63659-63660, 26070, 23781, 63661-63662, 34564, 63663-63668, 24118, 63669-63675, 24142, 63676-63698, 23555, 63699-63700, 63133, 24068, 63701-63702, 24238, 63703-63713, 24270, 24556, 63714-63716, 23564, 63717-63731, 25075, 63732-63737, 24043, 63738-63745, 24907, 24373, 63746, 24381, 63747-63754, 26404, 63755-63764, 26158, 63765-63776, 24465, 63777-63789, 23539, 63790-63791, 25337, 63792-63794, 27135, 63795-63800, 24524, 63801-63809, 24570, 63810-63831, 27383, 24635, 63832-63833, 25117, 63834-63836, 26551, 63837-63850, 26603, 24691, 63851-63857, 26855, 63858-63863, 24967, 63864, 35186, 63865, 25040, 63866-63869, 24774, 63870-63874, 24773, 63875, 23477, 63876-63879, 35257, 63880-63881, 25522, 63882, 24814, 63883-63886, 24734, 63887-63903, 25058, 63904-63908, 26426, 63909-63917, 23496, 63918-63925, 26390, 25216, 63926-63935, 24979, 25606, 23977, 63936, 26096, 63937-63943, 25003, 63944-63963, 25212, 63964-63970, 24304, 63971-63975, 25103, 63976-63997, 25542, 63998-64008, 12278, 64009-64019, 26482, 25221, 64020-64038, 27113, 24847, 64039, 23779, 64040-64054, 24661, 64055-64059, 25350, 64060, 61741, 64061-64073, 23605, 64074-64079, 25421, 64080, 25425, 64081-64090, 23993, 64091-64097, 26198, 25490, 64098-64099, 25500, 64100-64103, 25262, 64104-64107, 27220, 64108-64109, 25544, 64110-64113, 27095, 64114, 25246, 64115-64119, 25079, 64120-64127, 63161, 64128-64132, 25770, 25634, 64133-64136, 27072, 64137-64147, 25668, 64148-64156, 24622, 64157-64167, 25335, 64168-64174, 27101, 64175-64181, 23573, 64182-64189, 25768, 64190-64193, 25790, 64194-64203, 23857, 64204-64210, 23833, 64211-64239, 26107, 25122, 64240-64244, 63007, 64245-64250, 38833, 64251-64255, 6148, 64256-64260, 25974, 64261-64262, 24334, 64263, 25979, 64264-64265, 36336, 64266-64273, 25511, 24921, 64274-64284, 26106, 64285-64286, 26063, 26910, 64287, 26073, 64288-64297, 26111, 64298-64300, 26127, 64301-64302, 23552, 26424, 64303-64312, 23978, 64313-64330, 6062, 64331-64342, 26037, 27479, 64343-64348, 26284, 64349-64359, 24196, 64360-64374, 34329, 64375, 24586, 64376-64378, 26402, 64379-64382, 26417, 64383-64385, 25504, 64386-64387, 24955, 64388-64389, 27187, 26442, 64390-64396, 26465, 64397-64403, 26484, 64404-64418, 26540, 24463, 64419, 27332, 64420-64425, 24882, 26768, 24510, 64426-64442, 26420, 64443-64456, 24864, 64457, 26481, 64458-64469, 21871, 64470-64472, 24028, 64473-64476, 26696, 64477-64480, 24376, 64481-64484, 26548, 64485-64491, 26727, 64492-64498, 25063, 64499, 26742, 64500-64501, 25866, 64502-64509, 26767, 6034, 64510-64514, 26780, 64515-64518, 23717, 64519-64528, 24737, 61458, 64529-64542, 26262, 26857, 64543, 26992, 64544, 25373, 64545-64550, 26881, 64551-64556, 26436, 64557-64562, 26917, 64563-64564, 25417, 64565-64580, 25581, 26956, 64581-64588, 27418, 64589-64590, 26985, 64591-64601, 25748, 24541, 64602-64607, 26866, 64608-64629, 24053, 39071, 64630-64637, 27147, 64638-64645, 27183, 64646-64659, 25725, 64660-64662, 27255, 64663-64673, 27085, 27281, 64674-64686, 26134, 64687, 25453, 64688, 26139, 64689, 24981, 64690-64692, 25121, 64693, 26251, 64694-64696, 26018, 64697, 26706, 64698, 24208, 64699-64706, 25538, 64707-64712, 27432, 64713-64731, 27474, 64732-64739, 24913, 27364, 64740-64746, 27528, 64747-64759, 23463, 64760-64768, 23475, 64769-64777, 23493, 64778-64779, 23502, 25512, 64780-64782, 26701, 64783-64787, 23538, 64788-64795, 25392, 26844, 64796, 23586, 64797-64807, 23620, 64808-64811, 24777, 64812, 24554, 64813-64818, 23683, 64819, 39561, 64820, 57076, 64821-64828, 5834, 64829-64830, 26202, 23749, 64831-64863, 26989, 64864-64874, 24197, 64875-64888, 26870, 26940, 23866, 64889, 26816, 64890-64903, 57199, 64904-64914, 38596, 64915-64916, 24935, 64917-64918, 25095, 64919-64920, 26125, 25601, 64921-64934, 25758, 64935-64940, 25857, 64941, 24112, 64942-64946, 24879, 64947-64952, 26971, 64953-64956, 24147, 64957-64958, 25328, 64959-64963, 25939, 64964-64966, 25113, 64967, 25277, 24185, 64968-64973, 23704, 64974-64977, 24256, 24324, 64978-64987, 24249, 64988-64992, 24263, 64993-64995, 24277, 64996-64999, 25994, 65000-65010, 26334, 65011, 26501, 65012-65018, 24104, 65019, 24346, 65020, 24783, 65021-65028, 24075, 65029-65035, 24404, 65036-65047, 24435, 65048-65086, 24606, 65087-65093, 24538, 65094-65098, 24318, 65099-65103, 24589, 24596, 65104-65105, 24612, 65106-65133, 24682, 65134-65135, 24684, 65136-65145, 24987, 65146-65157, 27099, 65158-65165, 5835, 65166-65168, 27352, 65169-65172, 26086, 65173-65176, 24808, 65177-65203, 25729, 57617, 65204-65218, 24951, 24954, 65219-65222, 26012, 65223-65225, 37629, 65226-65230, 24687, 65231-65255, 26712, 65256-65266, 25087, 65267-65272, 26851, 65273-65275, 25432, 65276-65279, 25363, 65280-65284, 25142, 65285-65290, 25598, 65291-65292, 27074, 65293-65296, 25184, 65297-65304, 25203, 65305-65310, 25975, 65311-65317, 27526, 65318-65321, 26664, 65322-65323, 18265, 65324-65325, 25259, 65326-65329, 25891, 65330-65334, 25306, 25577, 65335-65369, 38513, 65370-65381, 25433, 65382-65392, 25483, 65393-65395, 25496, 65396-65401, 26889, 65402, 25161, 65403-65407, 25527, 65408-65411, 25537, 65412, 25165, 65413-65416, 25559, 65417-65431, 23727, 65432-65434, 25615, 65435, 26957, 25618, 65436-65481, 23973, 65482-65483, 25742, 65484-65493, 26987, 65494-65504, 24271, 65505-65506, 24551, 65507, 5854, 25622, 65508, 36186, 65509-65515, 25836, 25841, 65516-65522, 25856, 65523, 25860, 65524-65526, 25872, 65527-65530, 26679, 65531-65542, 36273, 65543-65548, 25199, 65549-65566, 25600, 26784, 65567-65570, 36360, 65571-65593, 26075, 65594-65595, 58604, 65596-65610, 27162, 65611-65613, 27274, 26124, 65614-65616, 25460, 26137, 65617-65628, 26324, 65629-65636, 23829, 26216, 65637-65647, 23998, 65648-65660, 23575, 65661-65681, 26351, 65682-65693, 26213, 26210, 65694-65697, 26392, 65698, 23956, 25482, 65699-65702, 26413, 26030, 65703-65711, 27116, 65712-65716, 36335, 65717-65718, 27120, 25701, 65719-65724, 26478, 65725-65741, 26528, 26530, 65742, 24248, 65743-65748, 26559, 65749-65787, 24390, 65788-65800, 26691, 65801-65810, 23824, 65811-65823, 27013, 65824-65834, 26793, 65835-65838, 27227, 65839-65848, 26835, 65849-65854, 23482, 65855-65857, 24845, 65858-65860, 24316, 65861-65863, 25984, 65864-65870, 17326, 65871-65886, 26955, 65887-65899, 26990, 65900-65904, 27000, 65905-65921, 24214, 27044, 27046, 65922-65926, 27063, 27065-27066, 65927-65950, 27122, 65951-65956, 27412, 25061, 65957-65964, 25208, 65965-65966, 27181, 65967-65970, 27201, 65971-65974, 24716, 65975, 26192, 65976-65978, 27248, 65979-65992, 27286, 65993-66004, 27322, 66005-66009, 27334, 27341, 26279, 66010-66019, 26320, 66020-66027, 27394, 66028-66034, 27421, 66035-66036, 41078, 66037-66039, 27435, 62979, 66040-66052, 26972, 66053, 27468, 66054-66066, 27511, 66067-66077, 24650, 66078-66085, 47269, 23662, 66086-66097, 23505, 26853, 66098-66099, 26690, 66100-66115, 25140, 66116-66125, 23617, 66126-66138, 26594, 66139-66141, 23657, 66142-66143, 24947, 23687, 66144-66146, 26222, 66147-66152, 23715, 66153, 23721, 25171, 66154-66155, 27373, 66156-66166, 23969, 25530, 26287, 66167, 6206, 66168, 25531, 66169, 24849, 66170-66175, 23809, 66176-66180, 26899, 66181-66203, 27069, 66204-66205, 26140, 66206-66207, 23890, 66208-66212, 26330, 66213-66215, 35365, 66216-66219, 23931, 66220-66242, 26278, 66243-66245, 24074, 66246, 24026, 66247-66249, 27455, 66250-66253, 24049, 66254-66257, 24056, 26939, 66258-66259, 59157, 66260-66261, 26290, 66262-66265, 24113, 66266-66275, 23870, 66276-66283, 26062, 39891, 66284-66292, 24276, 66293, 24194, 66294-66300, 24212, 26873, 66301, 25046, 66302-66310, 23579, 66311-66315, 24267, 66316-66335, 26586, 66336-66337, 25802, 66338-66341, 24128, 66342-66344, 60491, 66345-66346, 41005, 66347-66349, 26524, 66350-66356, 24858, 24370, 66357-66360, 24378, 66361-66362, 23757, 66363-66386, 24454, 66387, 24460, 66388-66401, 24502, 66402-66442, 15845, 66443-66448, 24670, 66449-66469, 24742, 66470-66482, 24723, 66483-66485, 35240, 66486-66488, 24799, 66489-66520, 24886, 66521-66535, 26147, 66536-66538, 24751, 66539-66540, 25106, 66541-66551, 24964, 66552-66553, 25440, 66554-66557, 26342, 66558-66562, 26511, 66563-66577, 25051, 66578-66579, 26026, 66580-66606, 25498, 66607-66608, 25148, 66609-66611, 27298, 66612-66613, 25648, 66614-66623, 25204, 66624-66625, 27447, 66626-66628, 26041, 66629-66632, 24539, 66633-66635, 25241, 66636-66637, 26188, 26490, 66638-66641, 25267, 66642-66648, 25028, 66649, 27025, 66650-66653, 24044, 66654-66669, 25410, 25464, 66670-66674, 24284, 66675-66678, 5825, 66679-66684, 26922, 66685-66690, 25406, 23523, 66691-66692, 25420, 66693-66707, 25847, 25477, 66708-66709, 25487, 66710-66712, 25501, 66713-66724, 26653, 66725-66734, 62684, 66735-66737, 25563, 66738-66755, 24574, 25200, 66756-66759, 27265, 66760-66770, 26758, 66771-66774, 25677, 66775-66778, 24655, 66779, 25660, 66780-66782, 25702, 66783-66784, 25715-25716, 66785-66799, 25753, 5724, 26670, 66800-66809, 23808, 66810-66829, 27319, 66830-66839, 26108, 25791, 66840-66845, 27327, 66846-66848, 23806, 66849-66854, 26406, 66855-66859, 25941, 25767, 24744, 66860-66863, 25949, 66864-66872, 37570, 66873-66880, 25985, 66881-66885, 26001, 66886-66893, 24944, 66894-66897, 26052, 66898-66899, 53657, 66900-66905, 26868, 26076, 30, 66906-66907, 26081, 66908-66918, 26116, 66919-66920, 26945, 66921-66922, 26543, 66923-66933, 27467, 66934-66940, 24685, 26625, 66941-66957, 26220, 66958, 26228, 26230, 66959, 5903, 66960-66967, 23901, 66968, 27390, 66969, 26270, 66970-66971, 26272, 66972-66973, 24014, 66974, 26281, 66975-66989, 27197, 66990-67003, 22973, 62679, 26369, 67004-67006, 26385, 67007-67018, 26769, 67019-67023, 26432, 67024-67029, 26087, 67030-67033, 25130, 67034-67038, 26483, 67039-67045, 26507, 67046-67051, 26519, 67052-67055, 23716, 67056-67058, 27475, 67059-67094, 39009, 67095, 25638, 67096-67105, 24936, 67106-67107, 26666, 24919, 67108-67110, 26050, 67111-67129, 26723, 67130-67136, 27209, 67137-67146, 27124, 67147-67148, 25360, 67149-67157, 23989, 67158-67163, 23515, 67164-67171, 26874, 67172-67198, 27518, 67199-67203, 24141, 67204-67208, 27043, 25497, 67209-67213, 25969, 67214, 24422, 67215-67219, 26765, 67220-67227, 27071, 67228, 34690, 67229, 27082, 37145, 67230-67232, 27091, 67233-67237, 62571, 67238-67249, 26838, 67250-67262, 25795, 67263-67268, 23891, 67269-67272, 26148, 67273-67279, 27245, 67280-67281, 26459, 67282-67291, 26180, 67292-67293, 27279, 67294-67306, 27309, 67307-67314, 27342, 67315-67320, 20894, 67321, 25805, 67322, 24587, 24121, 67323-67324, 24099, 67325-67330, 25656, 26925, 67331-67338, 6145, 27410, 67339-67342, 27429, 27433-27434, 67343-67349, 24943, 67350, 27454, 67351-67356, 27211, 67357-67381, 24451, 24714, 67382-67390, 26513, 67391-67399, 23462, 35893, 67400-67409, 23535, 67410, 25334, 67411-67430, 25150, 67431-67436, 23590, 67437-67444, 23619, 67445, 34247, 67446-67453, 25983, 67454-67463, 23677, 67464, 24780, 67465-67480, 24865, 67481-67491, 24200, 67492-67497, 25957, 67498-67500, 63413, 67501-67520, 25750, 67521-67529, 23612, 67530-67541, 24461, 67542, 24683, 67543-67546, 23881, 67547-67548, 24171, 23892, 67549-67552, 25535, 67553-67569, 23958, 67570, 23961, 23963, 67571-67576, 23643, 67577-67592, 27503, 67593, 24070, 67594-67603, 24109, 26604, 67604, 24689, 67605-67623, 24160, 67624-67632, 23574, 67633-67642, 23966, 67643-67662, 24246, 67663-67665, 24254, 24759, 67666-67675, 24302, 67676-67678, 23520, 23490, 67679-67688, 24374, 37370, 67689-67694, 23837, 67695-67700, 24411, 67701-67726, 23667, 67727-67728, 24507, 67729-67744, 26880, 67745-67759, 24008, 67760, 23514, 67761-67772, 24639, 67773-67774, 26468, 67775-67777, 27001, 67778-67785, 24468, 67786-67790, 24698, 67791-67802, 24466, 67803-67821, 24787, 67822-67829, 27133, 67830-67844, 23668, 67845-67846, 23439, 67847-67848, 24881, 67849-67856, 24902, 67857-67861, 24700, 67862-67866, 24949, 67867-67877, 24969-24970, 67878-67911, 25070, 67912-67914, 26112, 67915-67917, 25893, 26077, 67918-67920, 25094, 67921, 25097, 67922-67932, 25139, 67933-67942, 25175, 67943-67946, 24756, 25187, 67947, 25195, 67948-67949, 25914, 24709, 67950, 23646, 67951-67956, 25244, 67957-67961, 26682, 67962-67987, 25115, 24937, 67988-67993, 25358, 67994-68004, 27414, 68005-68009, 26261, 68010-68035, 26440, 27510, 68036-68057, 26257, 68058-68060, 24728, 68061-68067, 25597, 68068-68071, 24036, 68072-68075, 25637, 68076-68082, 62781, 68083-68089, 25275, 68090-68094, 25704, 68095-68097, 24813, 68098-68115, 25766, 68116-68117, 24528, 25902, 68118-68132, 25422, 68133-68135, 25828, 68136, 25833, 68137-68138, 25844, 25886, 68139, 24089, 27436, 68140, 25854, 68141-68142, 25865, 68143-68152, 6198, 68153-68156, 25906, 68157-68170, 25950, 68171-68173, 23971, 4826, 25964, 68174-68178, 23609, 68179, 23936, 68180-68182, 25989, 68183-68191, 26023, 68192-68196, 23867, 68197-68201, 37138, 68202-68208, 26067, 68209-68235, 26163, 68236-68237, 25614, 68238-68243, 26187, 68244-68247, 26196, 68248-68252, 62905, 68253-68259, 34355, 68260-68270, 26965, 68271-68278, 24402, 68279, 63301, 68280-68281, 26756, 68282, 25673, 68283-68289, 26345, 68290-68302, 24557, 68303, 25305, 68304-68315, 26591, 12039, 68316-68317, 38923, 68318-68328, 26452, 68329-68335, 26480, 68336-68356, 23828, 68357, 26533, 24747, 68358-68360, 26186, 68361-68370, 26574, 68371-68377, 25665, 25992, 68378-68381, 5795, 41724, 68382, 26630, 68383-68390, 26654, 68391-68396, 26675, 68397-68401, 7411, 68402-68409, 25261, 68410-68417, 26600, 68418-68421, 26755, 68422-68434, 26813, 68435-68438, 26821, 68439-68442, 25379, 68443-68462, 23786, 68463, 27496, 68464-68468, 23841, 68469-68491, 26983, 26581, 68492-68499, 27004-27005, 68500-68505, 24948, 27042, 68506-68508, 27462, 68509-68539, 26779, 27143, 68540, 26093, 68541-68543, 24762, 27165, 68544-68553, 27196, 68554-68565, 27235, 68566, 40189, 68567-68574, 27262, 68575-68578, 27272, 68579-68586, 26933, 68587-68599, 27317, 68600, 26456, 68601-68618, 26808, 68619-68626, 27125, 27369, 68627-68632, 27450, 68633-68639, 24279, 68640-68642, 25248, 68643-68645, 27480, 68646-68671, 27553, 68672-68687, 27211, 68688-68690, 57495, 68691-68698, 27977, 68699-68732, 675, 68733-68736, 15845, 68737-68771, 6061, 2300, 68772-68774, 67112, 68775-68800, 27637, 68801-68806, 7411, 68807-68821, 27650, 68822-68828, 27698, 68829-68860, 10, 68861-68874, 27736, 68875-68881, 27814, 68882-68895, 20359, 28036, 68896, 5834, 68897-68911, 15231, 68912-68918, 5356, 68919-68941, 37317, 68942-68948, 27574, 68949-68958, 5871, 68959, 27923, 68960-68967, 6348, 68968-68982, 23779, 68983-68990, 6360, 68991-69017, 27751, 69018-69029, 65992, 69030-69032, 62366, 69033-69035, 9776, 69036-69038, 38743, 69039-69042, 59082, 69043-69044, 4826, 69045-69049, 24970, 69050-69061, 27858, 69062-69076, 22561, 69077, 27547, 69078-69104, 27810, 69105-69134, 27588, 69135-69175, 27847, 69176-69187, 25334, 69188-69205, 27871, 69206-69230, 27572, 69231-69233, 38535, 69234-69246, 7052, 27953, 69247-69261, 28015, 69262-69269, 5903, 69270-69296, 38865, 69297-69320, 64526, 69321-69322, 27927, 69323-69342, 12039, 69343-69352, 58774, 69353-69355, 27984, 69356-69371, 5198, 69372-69381, 27965, 6206, 69382-69391, 27980, 69392-69397, 6154, 27598, 5455, 69398-69405, 5825, 69406-69422, 28000, 69423-69434, 34329, 69435-69440, 56935, 69441-69445, 57865, 69446-69462, 26077, 69463-69473, 27548, 26402, 69474-69483, 23344, 69484-69490, 60306, and 69491-69506, respectively, in order of appearance.

TABLE A4 in the Appendix is a full list of antibodies identified as unique to COVID-19 survivors. Among survivors the most antibodies categorize as Class 3 with other classes of antibodies having fewer reads. Table discloses SEQ ID NOS 2-19, 32-33, 20-21, 24238, 69507, 4864, 1241, 2568, 10139, 2855, 2300, 6244, 4772, 2745, 5347, 69508-69509, 2970, 4186, 2518, 18448, 9961, 26483, 69510, 965, 27034, 4828, 11277, 27802, 4932, 69511-69516, 24550, 69517, 26309, 843, 69518, 26995, 25469, 69519-69522, 3556, 9712, 4138, 69523-69525, 27703, 69526-69528, 2294, 69529-69530, 2368, 3503, 5240, 69531, 24443, 69532, 26941, 7368, 2635, 11177, 69533-69534, 1316, 720, 369, 394, 11105, 25518, 24107, 69535-69536, 25397, 1648, 9602, 69537-69539, 2975, 5100, 27148, 69540, 11683, 11324, 23601, 69541, 27391, 25178, 4610, 23901, 7252, 761, 5980, 69542, 2509, 24435, 2769, 69543, 5193, 9799, 69544-69546, 26686, 27954, 23537, 27736, 2429, 2494, 6711, 27728, 244, 3081, 31, 1878, 2410, 7328, 28032, 5290, 69547, 27158, 3784, 69548, 27586, 69549, 2589, 2304, 25184, 69550, 24700, 3364, 6606, 1429, 940, 4010, 5353, 10426, 4314, 69551, 24627, 69552, 1824, 4790, 4818, 69553, 27039, 69554, 2620, 24985, 1060, 7037, 186, 4695, 69555-69556, 6377, 2951, 3098, 6601, 3974, 5189, 5228, 11210, 10548, 326, 1354, 69557, 9752, 5106, 10038, 6993, 69558, 1143, 3897, 6977, 69559-69562, 412, 69563-69564, 2898, 27890, 2856, 4404, 9745, 24265, 1445, 2682, 6456, 69565, 3888, 69566-69567, 4082, 25974, 1921, 6654, 69568-69569, 225, 1526, 69570-69571, 1257, 11478, 24921, 27426, 5033, 27113, 3882, 3402, 26790, 7685, 459, 69572, 3625, 11554, 69573-69575, 1081, 3872, 69576-69577, 2164, 1399, 23569, 3569, 957, 27874, 25634, 69578, 10706, 69579, 23595, 69580, 27323, 26388, 69581, 2533, 24803, 69582, 2237, 69583, 6876, 69584, 1266, 25502, 242, 9727, 1541, 4897, 5095, 69585-69586, 3165, 27361, 4387, 26433, 27049, 526, 24524, 69587, 1831, 25852, 27491, 69588, 10607, 3847, 294, 26893, 69589, 24365, 25526, 9599, 69590, 78, 7387, 10397, 3382, 4755, 1837, 3929, 2097, 5118, 24840, 26629, 3990, 28008, 69591, 10113, 69592, 5218, 26710, 1228, 7035, 2272, 10948, 24908, 69593-69594, 2090, 119, 69595, 6467, 6706, 25690, 6782, 1466, 24789, 69596-69597, 24873, 6875, 69598, 7374, 69599, 2835, 4274, 11446, 27517, 69600, 10137, 69601, 25205, 27645, 69602-69603, 5069, 69604-69605, 26367, 69606-69607, 1025, 69608, 11244, 1514, 26459, 9768, 69609, 2967, 11117, 69610, 10813, 69611, 3545, 265, 69612-69614, 25454, 26919, 69615-69620, 24345, 69621-69624, 5017, 69625-69626, 10936, 69627, 2086, 10099, 69628-69631, 25970, 2457, 4470, 69632, 1865, 69633, 3541, 2748, 69634, 11273, 69635, 11279, 27619, 4516, 6483, 6754, 2345, 69636-69637, 11403, 4947, 69638, 10755, 16529, 1652, 24178, 25750, 24787, 5256, 69639, 4630, 69640-69641, 4359, 1150, 5340, 27268, 28014, 69642, 6507, 27717, 69643, 11026, 69644, 27914, 3885, 26162, 11459, 24408, 4027, 69645, 2423, 69646, 27394, 6557, 3340, 69647, 25406, 2585, 69648, 1888, 69649-69650, 26270, 10694, 69651, 27951, 24991, 69652, 26434, 69653, 3173, 414, 7151, 4995, 3582, 2579, 25562, 10004, 69654-69655, 1995, 6976, 26095, 4505, 6426, 3670, 7682, 7099, 11062, 69656, 6418, 261, 7157, 25405, 7090, 2728, 1248, 69657, 4188, 5322, 27597, 2018, 69658-69659, 23443, 26522, 1554, 26663, 27145, 69660, 27668, 24641, 389, 69661, 26207, 23784, 2965, 2866, 69662, 685, 69663, 7104, 10966, 7644, 3979, 69664, 6558, 26841, 69665, 2870, 69666, 6395, 27730, 69667, 24660, 69668-69669, 6237, 27832, 4005, 718, 69670, 4375, 69671, 24436, 69672, 1178, 23692, 69673-69674, 27075, 27719, 4385, 7396, 25101, 6622, 2476, 7548, 10447, 25841, 2167, 24782, 69675, 6920, 1188, 7501, 11095, 23563, 1403, 1312, 69676, 7546, 2452, 3629, 25933, 69677-69678, 24990, 25372, 69679, 2534, 27172, 2896, 2679, 9793, 5145, 6357, 4444, 69680, 24, 69681, 26740, 2339, 6449, 2279, 69682, 2353, 23, 5010, 69683-69684, 2054, 7047, 475, 69685, 22, 6457, 2674, 5164, 25, 24069, 24877, 69686, 7558, 69687, 27033, 6540, 2183, 2586, 856, 69688, 938, 9751, 3942, 6695, 2477, 2371, 3774, 1465, 23915, 1600, 69689-69690, 27, 3761, 26, 27884, 1810, 69691, 6279, 713, 9840, 28, 69692, 3350, 69693, 3886, 5364, 25787, 69694, 29, 762, 69695, 6462, 6803, 10551, 69696, 30, 1565, 69697-69704, 26012, 9776, 593, 69705-69707, 10643, 69708-69710, 7697, 4200, 27735, 69711-69712, 4688, 27711, 69713-69714, 24146, 69715-69719, 27623, 69720-69724, 1598, 69725-69728, 5359, 69729, and 69726, respectively, in order of appearance.

TABLE A5 in the Appendix is a full list antibodies identified as unique to COVID-19 non-survivors. Among non-survivors many of the top antibodies were categorized as Class 4 with other classes of antibodies having fewer reads. Table discloses SEQ ID NOS 34-53, 594, 69730, 23352, 3867, 13855, 69731-69732, 23203, 69733, 9074, 5470, 5417, 18923, 23163, 5654, 7863, 22947, 23401, 22000, 5640, 21229, 5899, 16588, 5562, 21706, 19542, 5466, 16865, 6155, 14171, 18735, 8511, 18817, 19448, 69734, 13565, 19582, 9351, 9187, 18984, 69735, 14468, 69736-69737, 17363, 69738, 6196, 69739-69740, 8595, 13140, 69741, 5691, 21352, 16875, 5444, 23041, 69742, 6106, 16640, 20246, 69743-69745, 20035, 14151, 69746-69748, 22262, 13804, 19352, 19509, 11931, 69749, 11951, 69750, 21859, 8344, 17495, 5767, 22200, 8867, 21535, 20016, 69751, 22341, 12798, 69752, 15407, 14223, 12114, 22031, 20914, 17311, 20654, 17493, 15574, 19077, 69753, 15451, 69754, 22030, 23268, 16846, 17069, 14959, 69755, 23298, 69756, 15358, 69757, 12562, 7811, 69758, 16936, 18937, 22319, 16085, 18361, 17857, 14523, 69759-69761, 14777, 69762-69763, 14389, 69764, 5400, 69765, 21736, 17355, 5882, 69766, 20870, 19317, 69767, 18914, 15369, 14855, 12058, 14016, 69768, 13287, 15763, 21753, 21824, 14407, 8932, 20535, 15595, 69769, 8446, 69770, 15178, 69771-69772, 15176, 22183, 69773, 21649, 16432, 5465, 22226, 8288, 12462, 20338, 20046, 23343, 69774, 5953, 16187, 14287, 8097, 69775, 20287, 23141, 69776, 11892, 69777-69778, 17317, 8050, 12282, 23131, 22785, 8465, 12518, 21217, 16102, 15919, 19730, 14452, 20116, 69779, 21534, 5376, 69780, 8200, 69781, 21092, 69782-69783, 16702, 16452, 20117, 14018, 21929, 19879, 69784-69785, 5411, 6049, 69786, 21115, 16655, 18697, 20778, 69787, 14845, 8708, 17679, 9433, 16206, 69788-69789, 20325, 14012, 17671, 11858, 8418, 6014, 8491, 69790-69791, 15599, 7816, 13466, 19182, 69792, 17861, 12333, 8063, 19964, 69793, 17022, 12636, 18438, 5762, 69794-69795, 20769, 69796, 14390, 69797, 9483, 21122, 18147, 13295, 12499, 18870, 69798-69799, 14550, 20566, 69800, 12864, 69801-69806, 18062, 69624, 18579, 69807-69810, 13181, 14233, 18363, 69811-69816, 16943, 8567, 69817, 23204, 9147, 69818-69819, 9242, 14008, 6057, 69820, 16126, 6046, 69821, 22769, 22167, 15499, 69822, 16162, 69823, 5661, 69824, 19175, 19513, 13920, 69825, 9371, 12880, 12719, 22432, 69826, 21159, 69827, 21212, 9132, 18462, 69828, 12723, 69829, 22970, 20314, 8659, 19050, 8456, 12629, 14812, 16275, 69830-69833, 20061, 69834-69835, 16003, 19745, 15528, 69836, 23265, 13972, 19842, 69837-69838, 15755, 69839, 16243, 5675, 5484, 69840, 15692, 5605, 17213, 13434, 13886, 69841-69843, 21794, 69844, 23418, 11814, 23281, 69845, 23139, 9501, 20776, 12557, 69846, 20013, 23144, 5685, 6068, 22562, 23060, 18442, 22773, 69847, 21356, 69848, 23055, 13257, 17482, 69849, 17271, 19150, 23171, 5750, 3960, 14467, 13435, 69850, 23383, 13601, 22566, 23121, 5436, 12686, 69851-69853, 22452, 69854, 18286, 5586, 69855, 19781, 69856-69857, 19086, 69858-69861, 23179, 69862, 8579, 69863-69865, 23167, 69866-69870, 18523, 69871, 17640, 69872, 8459, 69873-69874, 23422, 69875, 22976, 69876-69881, 5595, 69882-69899, and 69766, respectively, in order of appearance.

### DETAILED DESCRIPTION OF THE INVENTION

### Industrial Applicability

COVID-19. When looking at COVID-19 patients across several time points, the inventors identified what was shown to be a critical intervention point in patients with reduced levels of COVID-19 antibodies. A cocktail of antibodies from TABLE 6 may be beneficial in treating COVID-19 patients, as a treatment in addition to COVID-19 vaccinations.

Antimicrobial-resistant bacteria cause almost five million deaths each year. Global burden of bacterial antimicrobial resistance in 2019: A systematic analysis (2022). Early identification of the causative pathogen and its antibiotic resistance pattern is central to infection management by focusing on antimicrobial administration. Typical clinical practice in patients with infections would benefit by starting treatment with broad-spectrum antibiotics as early as possible.

Despite the benefit of broad-spectrum antibiotics in reducing infection mortality, there are also negative consequences. Broad-spectrum antibiotics are costly and labor-intensive. They increase the risk of Clostridioides difficile colitis and select new, antibiotic-resistant pathogens.

Culturing the site of infection identifies the pathogen and its associated antibiotic resistance but can take days to generate actionable information. Antibiotics administered before sample acquisition can reduce culture yields.

Faster pathogen identification for severe infections. Sepsis causes one out of five deaths in the world. Rudd et al. (2020). The diagnosis of septic infection is a significant challenge for sepsis care. Duncan, Youngstein, Kirrane, & Lonsdale (2021). This invention provides better diagnoses of bacterial infections and associated antimicrobial resistance to improve outcomes.

The Surviving Sepsis Campaign standardized treatment for sepsis that includes blood cultures before broad-spectrum antibiotics and initiation of antibiotics within one hour. See Evans et al. (2021). In a multivariate analysis of factors impacting mortality in patients with septic shock, the time to begin antibiotic treatment was the most impactful variable. Kumar et al. showed this impact, reporting a 79.9% survival in septic shock patients with antibiotics in the first hour and a reduction of 7.6% for every hour delay. Kumar et al. (2006). Vazquez-Guillamet et al. determined that the number needed to treat with antibiotics to save one life was five. Vazquez-Guillamet et al., (2014). Faster pathogen identification improves sepsis outcomes by guiding antibiotic selection. Current methods take too much time, such as days. Sepsis kills in hours.

This invention provides diagnostic tests for three pathogens that cause bacteremia and three pathogens that cause pneumonia to shorten the time to pathogen-specific treatment for diseases such as sepsis.

Antimicrobial resistance is a significant health problem. Across the world, antimicrobial-resistant bacteria cause almost five million deaths each year. Global burden of bacterial antimicrobial resistance in 2019: A systematic analysis (2022 Antimicrobial-resistant bacteria cause more than 100,000 deaths in the United States with costs of over $21 billion. With each antibiotic, resistance follows shortly after that. See Clatworthy, Pierson, & Hung (2007). The United Nations convened a high-level meeting on Antimicrobial Resistance on Sep. 21, 2016, that included statements by global leaders such as Secretary-General Ban Ki-moon: “Drug resistance imposes huge costs on health systems and is taking a growing—and unnecessary—toll in lives and threatening to roll back much of the progress we have made.” Locally the trend is increased in resistance also increasing among many pathogens. Kassakian & Mermel (2014).

Importance of phenotypic antibacterial susceptibility. Bacteria have multiple antimicrobial resistance mechanisms and are easily transferred, resulting in pathogens with extensive resistance profiles. Harbottle, Thakur, Zhao, & White (2006). Despite advances in genomic testing, resistance genes found in DNA do not always correlate with phenotypic resistance. Bortolaia et al. (2020). Some computational approaches were suggested to handle this data. Bortolaia et al. (2020). Reasons for the lack of correlation between genomic data and resistance phenotypes include lack of transcription and DNA not associated with a living cell. Using RNA sequencing data allows for a better correlation between the genomic data and the expression of resistance. Using RNA data identifies only genes actively being transcribed, thereby measuring gene expression levels.

Facilitate antimicrobial stewardship. Antibiotic stewardship was suggested as a method to combat resistance. Broad-spectrum antibiotics, although appropriate initially, have an adjusted increased mortality risk. Webb et al. (2019). Other studies specifically state that unnecessary broad-spectrum antibiotics increase the risk of in-hospital death when no resistance is identified. Rhee et al. (2020). Unnecessary broad-spectrum coverage is used in 67.8% of patients. Rhee et al. (2020). De-escalation is important because new resistance emerges each day of inappropriate antibiotic exposure. Teshome et al. (2019). In the hospital setting, empiric therapy is only de-escalated 16% of the time. De Bus et al. (2020). Costs are reduced when de-escalation is used. Seok, Jeon, & Park (2020). Narrowing antibiotics reduces labor in the intensive care unit. Mei-Sheng, Riley & Olans (2021). A diagnostic test that rapidly identifies a pathogen and its antimicrobial resistance should assist in antimicrobial stewardship.

In the twelfth embodiment above, PCRs informed by a large dataset are performed in four hours, yielding direct from blood results independent of culture. Al-Hasan, Winders, Bookstaver, & Justo recommend directly assessing stewardship programs rather than looking for adverse events. Al-Hasan, Winders, Bookstaver, & Justo (2019). With faster bacteria identification, serial testing could assess treatment efficacy. Serial testing could be an additional metric in stewardship programs as antibiotics could be stopped sooner.

Similarities between COVID-19 and sepsis immunopathogenesis and pathophysiology. Similarities between COVID-19 and sepsis conditions include multiple organ dysfunction, immunosuppression, disseminated intravascular coagulation (DIC) and abnormal coagulation, elevated bilirubin, hypoxia, reduced glomerular filtration rate, and hypoalbuminemia, and acute respiratory failure and cytokine storm. Olwal et al., Front. Immunol. (February 2021); Lu et al., Front. Immunol. (August 2022). Recommendations for management of severe inflammatory response syndrome (SIRS) in sepsis may benefit severe COVID-19 patients. Olwal et al., Front. Immunol. (February 2021); Lu et al., Front. Immunol. (Aug. 31, 2022). In a preliminary set of assays, gene expression profiles of COVID-19 and sepsis patients were obtained from the Gene Expression Omnibus (GEO) database and compared to extract common differentially expressed genes (DEGs). Based on enrichment analysis of common DEGs, many pathways closely related to inflammatory response were observed, such as Cytokine-cytokine receptor interaction pathway and NFκB signaling pathway. Protein-protein interaction networks and gene regulatory networks of common DEGs were constructed, and the analysis results showed that ITGAM may be a potential key biomarker base on regulatory analysis. Potential therapeutic agents, including progesterone and emetine, were screened through drug-protein interaction networks and molecular docking simulations.

Unmapped reads identify bacterial RNA with deep RNA sequencing. In the initial assessment of RNA sequencing data, the reads are aligned to the genome of the species the sample came from, commonly the human genome. Unmapped reads can account for up to 20% of the data. These data are typically discarded. In the samples of humans with critical illness, there are more unmapped reads (˜35%). Monaghan et al. (2021). The Read Origin Protocol (ROP) (Mangul et al. (2018)) and Kraken (Wood, Lu, & Langmead (2018)) have been developed to determine the origin of unmapped reads. The Read Origin Protocol analysis of multiple data sets mapped 99.9% of all reads. The data that were typically discarded were analyzed in a seven-step process. One step is of particular interest because of the relevance to the patient population in this work: bacterial reads. Using ROP, or more recently Kraken2, bacterial RNA was identified in the blood samples of patients with sepsis, which mapped to the bacteria found in blood culture. RNA sequencing data can inform primer design to produce better diagnostic tests.

Diagnostic solution. This invention leverages a large data set of unmapped reads from patients diagnosed with infections by the gold standard of bacterial culture. This deep RNA sequencing data suggest PCR primers to identify pathogens, such as S. aureus, E. coli, P. aeruginosa, and H. influenza, and clinically relevant resistance genes. These tests are culture-independent and allow for direct from blood testing where PAXgene tubes stabilize the RNA. The PAXgene Blood RNA Tubes (QIAGEN, MD, USA; Cat. No./ID: 762165) are used for in vitro diagnostic testing (IVD). Sensitivity and specificity align with the requirements of the FDA as it relates to molecular diagnostic tests. Because RNA is used, phenotypic identification is done better than past attempts at DNA sequencing. See BMJ Global Health, 5(11) (2020).

Conceptual innovation. Reads that do not align to the genome of interest (human in these assays) are typically discarded. In this invention, the unmapped reads are the focus of the investigation to identify novel PCR targets in patients with bacterial infections.

Deep RNA sequencing, greater than 100 million reads, allows for identifying bacterial RNA in the blood of patients with infections.

Focusing on RNA rather than DNA improves phenotypic correlation with antimicrobial resistance.

Globin and ribosomal RNA are reduced to enhance the identification of the bacterial genes expressed.

Clinical management is guided by these RNA-based PCR tests designed to identify target genes directly affecting treatment decisions.

The RNA-based PCR tests are developed with the specific objective of rapid dissemination to clinical microbiology laboratories.

Technical innovation. Unmapped reads from deep RNA sequencing are an untapped resource of new information. Typically, 30% of reads are unmapped, so deep RNA sequencing of 100 million reads contains 30 million reads for further analysis.

The invention uses analytical algorithms that include mapping reads to genomes created for each pathogen based upon standard features across large numbers of strains.

The computational analysis is enhanced with customized algorithms and improved computing power, shortening the time to primer identification.

Workflow is optimized and automated to protect RNA, including PAXgene tubes for phlebotomy.

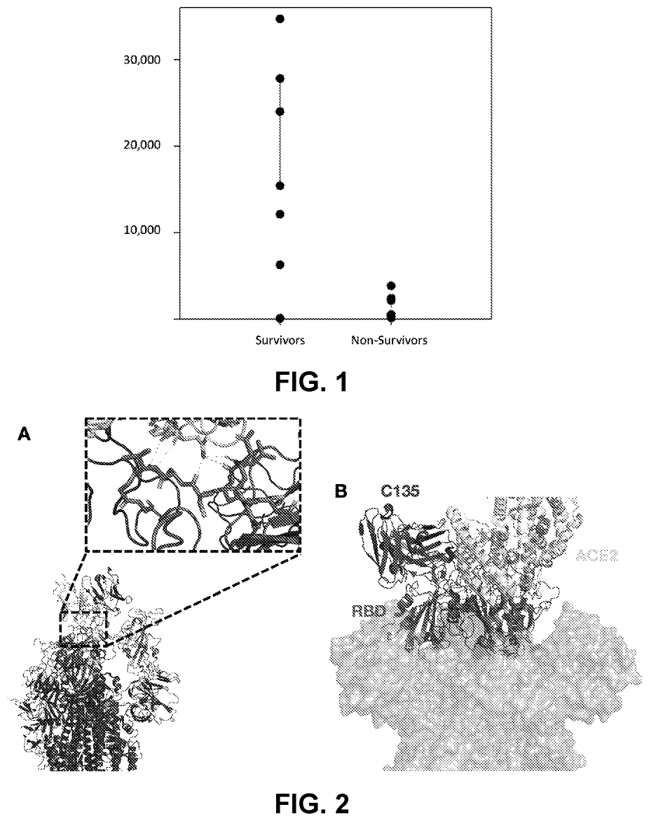

Deep RNA sequencing identifies pathogen RNA and informs PCR primer. Preliminary data was created using RNA sequencing from COVID-19 patients in the intensive care unit. Data from the deep RNA-sequencing study indicated limited regions of the viral genome were detected in the bloodstream of critically ill COVID-19 patients. This information was used to design primers to validate the sequencing results with a different methodology. cDNA generated from patients' RNA was subjected to quantitative, real-time, reverse transcriptase PCR using two sets of primers for the N gene. One primer pair corresponded to the peak of sequencing reads. Another primer pair was selected at a different site of the gene. See FIG. 1A. Using standard SYBR green methodology, amplicons for the peak N sequence were identified in all tested samples. By contrast, a template corresponding to the off-peak sequence was detected in only nine of the fifteen patients. When present, the abundance of the off-peak sequence was 4 to 16-fold lower than the peak sequence. See FIG. 1A.

This work is important because detecting SARS-CoV-2 in the blood was difficult. Yan, Chang, & Wang (2020).

In one aspect, unmapped RNA reads from patients with infections that align with pathogens can inform a better diagnostic test. The invention uses deep sequencing (>100 million reads) to identify the most highly expressed RNAs in the blood of patients with bacteremia or pneumonia. Cultures and antibiotic susceptibility testing are performed as the gold standard. RNA sequences from pathogens in transcription analysis are typically discarded because they would not align with the human genome. The inventors identify these “unmapped reads” in patients' blood and align them to a custom “genome” derived from the pathogens of interest to identify the causative organism. RNA that aligns with resistance genes is also specified. The commonly identified and organism-specific sequences are the template for designing oligonucleotide primers for use in RT-qPCR tests.

TABLE 3

| | Method | Result | Impact |
|---|---|---|---|
| A1a and A1b | Deep RNA sequencing | PCR is validated to | Common bacteria |
| | identifies RNA to | test for bacteremia | causing bacteremia |
| | target pathogens in | caused by S. | and pneumonia are |
| | infected patients | aureus, E. coli, P. | identified more |
| | with bacteremia due | aeruginosa in | quickly, allowing |
| | to S. aureus, E. coli, | samples from | faster administration |
| | P. aeruginosa to | patients with and | of tailored antibiotics, |
| | create PCR | without blood | improving survival, and |
| | | infections | enhancing antimicrobial |
| | | | stewardship. |
| A2a and A2b | Deep RNA sequencing | PCR is validated to | |
| | identifies RNA to | test for pneumonia | |
| | target pathogens in | caused by S. aureus, | |
| | infected patients | P. aeruginosa, or | |
| | with pneumonia due | H. influenzae in | |
| | to S. aureus, P. | samples from patients | |
| | aeruginosa, or H. | with and without lung | |
| | influenzae to create | infections | |
| | PCR | | |
| A3a and A3b | Deep RNA sequencing | PCR is validated to | Infections due to |
| | identifies RNA to | test for clinically | resistant organisms |
| | target related to | relevant resistance | are identified quickly. |
| | clinically relevant | genes to S. aureus, | Antibiotics are used |
| | resistance genes to | E. coli, P. aeruginosa, | immediately, reducing |
| | create PCR | or H. influenzae in | the unnecessary use |
| | | samples from patients | of broad-spectrum |
| | | with and without | antibiotics. |
| | | resistant infections | |

RNA sequencing can be a medically useful tool for personalizing the care of sepsis patients. With these advances, this tool will be used by clinicians in the Intensive Care Unit caring for sepsis patients.

The improvements listed above are also useful for designing better platforms and reagents for direct from blood, without the need for culture, reverse transcriptase polymerase chain reaction (RT-qPCR) tests for bacteria. QIAGEN (Germantown, MD, USA) is a manufacturer of platforms and reagents for RNA isolation and sequencing. Abbott (Abbott Park, IL, USA), Cepheid (Sunnyvale, CA, USA), Thermo Fisher Scientific (Waltham, MA, USA), and ELITech Group (Puteaux, FR) are also manufacturer of platforms and reagents for RNA isolation and sequencing.

### Definitions

For convenience, the meaning of some terms and phrases used in the specification, examples, and appended claims, are listed below. Unless stated otherwise or implicit from context, these terms and phrases shall have the meanings below. These definitions aid in describing particular embodiments but are not intended to limit the claimed invention. Unless otherwise defined, all technical and scientific terms have the same meaning as commonly understood by a person having ordinary skill in the biomedical art. A term's meaning provided in this specification shall prevail if any apparent discrepancy arises between the meaning of a definition provided in this specification and the term's use in the biomedical art.

Acute respiratory distress syndrome (ARDS) has the medical art-recognized meaning. ARDS is a type of respiratory failure characterized by rapid onset of widespread inflammation in the lungs. Symptoms include shortness of breath, rapid breathing, and bluish skin coloration. Causes may include sepsis, pancreatitis, trauma, pneumonia, and aspiration.

Aldo/keto reductase gene has the biomedical art-recognized meaning.

Alternative splicing (AS) has the biomedical art-recognized meaning. RNA splicing is a fundamental molecular function that occurs in all cells directly after RNA transcription but before protein translation, in which introns are removed and exons are joined. Alternative splicing or alternative RNA splicing, or differential splicing, is a regulated process during gene expression that results in a single gene coding for multiple proteins. Exons of a gene can be included within or excluded from the final, processed messenger RNA (mRNA) produced from that gene. The proteins translated from alternatively spliced mRNAs can contain differences in their amino acid sequence and, often, in their biological functions.

Base R is an R-based computer program.

Mann Whitney U tests have the statistical art-recognized meaning. The Mann-Whitney U test (also called the Mann-Whitney-Wilcoxon (MWW), Wilcoxon rank-sum test, or Wilcoxon-Mann-Whitney test) is a nonparametric test of the null hypothesis that it is equally likely that a randomly selected value from one population is less than or greater than a randomly selected value from a second population. This test can investigate whether two independent samples were selected from populations having the same distribution.

mountainClimber is a cumulative-sum-based approach to identifying alternative transcription start (ATS) and alternative polyadenylation (APA) as change points. Unlike many existing methods, mountainClimber runs on a single sample and identifies multiple ATS or APA sites anywhere in the transcript. Cass & Xiao, Cell Systems, 9(4), 23, 393-400.e6 (October 2019).

Next Generation Sequencing (NGS) has the biomedical art-recognized meaning. NGS technology is typically highly scalable, allowing the entire genome to be sequenced at once. Usually, this is accomplished by fragmenting the genome into small pieces, randomly sampling for a fragment, and sequencing it using various technologies.

Principal Component Analysis (PCA) has the biomedical art-recognized meaning. The principal component analysis is a statistical procedure that uses an orthogonal transformation to convert a set of observations of possibly correlated variables (entities, each of which takes on various numerical values) into a set of values of linearly uncorrelated variables called principal components.

Read has the biomedical art-recognized meaning of reading sequencing results to determine nucleotide base structure.

Read origin protocol (ROP) has the biomedical art-recognized meaning of a computational protocol that aims to discover the source of all reads, including those originating from repeat sequences, recombinant B and T cell receptors, and microbial communities. The Read Origin Protocol was developed to determine what the unmapped reads represented. Mangul al., Genome Biology 19, 36 (2018). Recent development of Read Origin Protocol (ROP) has demonstrated that unmapped reads align to bacterial, viral, fungal, and B/T rearrangement genomes.

Sepsis has the medical art-recognized meaning of a life-threatening condition that arises when the body's response to infection injures its tissues and organs. See Bone et al., Chest, 101, 1644-1655 (1992); Singer et al., JAMA, 315, 801-810 (February 2016).

STAR aligner is the Spliced Transcripts Alignment to a Reference (STAR), a fast RNA-seq read mapper, with support for splice-junction and fusion read detection. STAR aligns reads by finding the Maximal Mappable Prefix (MMP) hits between reads (or read pairs) and the genome, using a Suffix Array index. Different parts of a read can be mapped to different genomic positions, corresponding to splicing or RNA-fusions. The genome index includes known splice-junctions from annotated gene models, allowing for sensitive detection of spliced reads. STAR performs local alignment, automatically soft clipping ends of reads with high mismatches. Dobin et al., STAR: Ultrafast universal RNA-seq aligner. Bioinformatics, 29(1), 15-21 (January 2013).

Therapy that is efficacious for treating COVID-19 patients has the biomedical art-recognized meaning. In addition to the therapies disclosed in this specification, therapies efficacious for treating COVID-19 patients are disclosed from the United States Center for Disease Control. Among the therapies efficacious for treating COVID-19 patients are nirmatrelvir with ritonavi (Paxlovid®), remdesivir (Veklury®), bebtelovimab, and molnupiravir (Lagevrio®). New therapies are in the process of being discovered and recommended. Also, therapies efficacious for treating COVID-19 patients include treatments for sepsis, such as corticosteroids, insulin, drugs that modify the immune system responses, and painkillers or sedatives.

Treatment for sepsis has the medical art-recognized meaning. Sepsis is treatable, and timely implementation of targeted interventions improves outcomes. The Mayo Clinic informs the public that several medications are used in treating sepsis and septic shock. They include antibiotics. Broad-spectrum antibiotics, which are effective against various bacteria, are usually used first. After learning the results of blood tests, a doctor may switch to a different antibiotic targeted to fight the specific bacteria causing the infection. Other medications include low doses of corticosteroids, insulin to help maintain stable blood sugar levels, drugs that modify the immune system responses, and painkillers or sedatives.

V(D)J recombination has the biomedical art-recognized meaning. V(D)J recombination occurs in developing lymphocytes during the early stages of T and B cell maturation, involves somatic recombination, and results in the highly diverse repertoire of antibodies/immunoglobulins and T cell receptors (TCRs) found in B cells and T cells, respectively.

Whippet (OMICS_29617) is a program that enables the detection and quantification of alternative RNA splicing events of any complexity with computational requirements compatible with a laptop computer. Whippet applies the concept of lightweight algorithms to event-level splicing quantification by RNAseq. The software can facilitate the analysis of simple to complex alternative splicing events that function in normal and disease physiology. Alternative splicing events with high entropy are identified using Whippet. Sterne-Weiler et al., Molecular Cell, 72, 187-200.e186 (2018).

Unless otherwise defined herein, scientific, and technical terms used with this application shall have the meanings commonly understood by persons having ordinary skill in the biomedical art. This invention is not limited to the particular methodology, protocols, and reagents, etc., described herein and as such can vary.

The disclosure described herein does not concern a process for cloning humans, processes for modifying the germ line genetic identity of humans, uses of human embryos for industrial or commercial purposes or processes for modifying the genetic identity of animals which are likely to cause them suffering with no substantial medical benefit to man or animal, and also animals resulting from such processes.

Guidance from Materials and Methods

A person having ordinary skill in the biomedical art can use these materials and methods as guidance to predictable results when making and using the invention:

Deep sequencing methods. The concept of diagnostics is analogous to using a fishing lure to find a single protein, gene, or RNA sequence. The invention provides a specific concept, using a fishing net to obtain all the RNA data in a sample, and use computational biology to better sort through all the data (fish) to identify patients with sepsis and the bacteria causing the immune response. The invention provides an initial diagnostic for sepsis that can also monitor the indicia of treatment and recovery (bacterial counts reduce, physiology returns to steady state). The invention can be used for many other hospital conditions, particularly those needing an intensive care unit stay with the attendant risk of bacterial infection, such as trauma, stroke, myocardial infarction, or major surgery.

In producing the listed embodiments, one of ordinary skill in the biomedical art uses the following steps: The first step is for one of ordinary skill in the biomedical art to obtain RNA sequencing from a body sample, such as a bodily fluid sample, for example, blood. The target is 100,000,000 reads/sample. The second step is for one of ordinary skill in the biomedical art to align the RNA sequencing data (reads) to a genome of interest, such as a human genome. The third step is to select the un-mapped reads and analyze the reads using a Read Origin Protocol (ROP). From the ROP, one of ordinary skill in the biomedical art identifies COVID-19 variants that are present in the sample. The fourth step is to identify the T/B cell epitopes present in the samples.

Study design, population, and setting. Intensive care unit patient-participants at a single tertiary care hospital were enrolled after they or their surrogates provided informed consent (Institutional Review Board Approval #411616). SARS-CoV-2 infection was based on positive PCR from the nasopharynx. Patients were followed until discharge or death and clinical information was collected prospectively. Blood samples were collected on day 0 of intensive care unit admission. Some patients also had samples collected on day 3 of intensive care unit admission.

RNA extraction, sequencing, data protection and quality assurance. Blood was collected directly from the patient into PAXgene tubes (Qiagen, Germantown, MD, USA) to stabilize the RNA. The tubes were then stored as described by the manufacturer. Cohorts of samples (all from day 0 and all from day 3) were sent to Genewiz (South Plainfield, NJ, USA) for RNA extraction, ribosomal RNA depletion, and sequencing on Illumina HiSeq machines with greater than 100 million reads per sample.

To ensure security of the genomic data to the United States Health Insurance Portability and Accountability Act (HIPAA) standards, the raw files were returned on password-protected external hard drives. All analysis was done on servers within the hospital firewalls. As previously described, the inventors assessed data quality using FastQC. See Monaghan et al., medRxiv (2021). Sequencing data was aligned using STAR aligner with standard parameters. Unmapped reads were included in the output.

Identification of antibodies. Alignment files were then parsed for reads mapping to the V(D)J locus using ImReP9 to identify novel antibodies produced by the patients with active COVID-19 disease requiring intensive care unit admission. The resulting CDR3 sequences were then compared across survivors and non-survivors. Only sequences that appeared in every patient in each group were considered distinct to that group. Comparison was done using a NCBI Blast program, with a threshold of 66% length match and 70% sequence match. Querying sequences by time point, across time points, further filtered blast output and survivors vs. non-survivors by time point and across time points.

Classification of CDR3 regions and model of neutralizing Fab. The patient-derived light chain CDR3 sequences were aligned to known CDR3 regions of previously identified neutralizing antibodies. See TABLE A1. CDR3 sequences with similar primary sequences to the CDR3 classifications were placed into categories. When the primary sequence did not align to any of those sequences, the CDR3 was left unclassified.

The model of the Fab binding to the spike protein described in FIG. 2 was created from the Cryo-EM structure of C110 NAb binding to the spike Protein (7K8V). The C110 NAb CDR3 sequence was mutated to CQQYNNYWAF (SEQ ID NO: 1) in Pymol (Schrodinger, Inc.) and the rotamers were optimized. The mutated light chain and RBD were isolated. The binding interface was optimized using the GalaxyRefineComplex Server. The lowest energy model was then selected for further analysis. The optimized light chain and RBD were then inserted back into the 7K8V structure and analyzed in Pymol for hydrogen bonding networks.

Statistical analysis. SigmaPlot 14.5 (Sysstat Software) was used for analysis. T-tests were used to compare survivors and non-survivors, but paired t-tests were used to compare across time points. Alpha was set at 0.05.

Human subjects. The inventors are enrolling patients in the Intensive care units with sepsis and sending their blood for deep RNA sequencing. After Institutional Review Board approval, patients are recruited for this research program from the emergency department and hospital patients when blood cultures are ordered. Through alerts from the electronic health record (EPIC), research assistants are notified of when blood cultures are ordered. Patients have consented before the collection of the blood culture. Samples are drawn in collaboration with the phlebotomy service and the bedside nurse. Blood is collected in two PAXgene tubes, 5 mL of blood, and stored in an −80° C. freezer until RNA is isolated for sequencing. As a test of feasibility, the last six months of data in the hospital were reviewed, and many samples were available. Over the six-month time course, 2,453 patients had blood cultures drawn in the emergency department, and 602 patients had blood cultures drawn in the Intensive Care Units. Blood is also collected from patients who undergo bronchial alveolar lavage (BAL) in the Intensive Care Unit to diagnose pneumonia. Samples are collected before the bronchial alveolar lavage and stored as described. Over the six-month time course, 46 patients had BAL samples obtained in the emergency department and 51 patients had bronchial alveolar lavage samples obtained in the Intensive Care Units.

RNA isolation and sequencing. Blood from patients are collected using the PAXgene tubes (PreAnalytix, Switzerland). All samples require at least 1400 nanograms of RNA for deep sequencing. With the PAXgene system, one routinely obtains >3000 nanograms. After RNA samples are processed, they are sent out for RNA sequencing. Because of the high concentration of globin and ribosomal RNA in blood samples, these samples are then further processed at the sequencing company to reduce globin RNA and human ribosomal RNA. This optimizes the yield of clinically relevant reads. Each sample are sent out for deep RNA sequencing with a goal of 100 million reads per sample.

RNA sequencing are done on non-CLIA machines because this data is not used in clinical practice. The vendor has Clinical Laboratory Improvement Amendment (CLIA) certified machines to allow for ease of translation in future studies. Not all the blood samples collected are sent for deep RNA sequencing. One of the two PAXgene tubes are kept for the PCR tests.

Sample size calculation. Patients with bacteremia are compared to patients without bacteremia to identify targets for the creation of the PCR. Based upon the positive culture rate (TABLE 4), the inventors would need to collect 2200 blood cultures to obtain fifty that are positive for Staphylococcus aureus. These rates are for all samples. The inventors are targeting the collection for the Emergency Department and the Intensive Care Units, so the positive rate are higher. A total of 3500 samples are obtained to get a representation of each type of organism. The institution averages 3000 blood cultures in the Emergency Department and the Intensive Care Units every six months. This testing results in at least sixty patients with S. aureus, thirty with E. coli, and ten with P. aeruginosa. All samples with a corresponding positive blood cultures for these three pathogens are sent for deep RNA sequencing. The inventors also send samples for RNA sequencing with a corresponding positive blood culture, including those judged to be contaminants, for a total of approximately 135, with an additional 115 of samples from patients with negative blood cultures. This process would result in 350 samples sent for RNA sequencing for EXAMPLE 2. The second PAXgene tube drawn on these patients is used to verify the PCR tests. Patients with pneumonia are compared to patients without pneumonia to identify targets for the creation of the PCR. Based upon the positive culture rate (see TABLE 4), the inventors would ideally collect all patients with a BAL from the Emergency Department or Intensive Care Unit. Over six months, this process would include about 100 patients and would result in eleven with S. aureus, ten with P. aeruginosa, and four with H. influenza. The inventors collect eighteen months of samples to obtain about 300 blood tubes to sequence for the pneumonia section of the invention. Because two pathogens are being studied, these patients have complementary BAL and blood cultures sent simultaneously. Resistance genes are identified using the same samples collected.

TABLE 4
Microbiological Culture Positivity Rates

| Bacteria | Blood Culture | BAL |
|---|---|---|
| Staphylococcus aureus | 2.2% | 11% |
| Escherichia coli | 1% | N/A |
| Pseudomonas aeruginosa | 0.3% | 9.5% |
| H. influenza | N/A | 3.7% |

Assessment of clinical information. RNA sequencing data are interpreted with clinical data collected from the electronic medical record including endpoints such as mortality, Intensive Care Unit length of stay, hospital length of stay, SOFA score (Shankar-Hari et al. (2016)), ventilator days, renal failure, ARDS (Ferguson et al. (2012)). Culture data are based upon the test results in the microbiology lab and are the gold standard. Clinical response to antibiotics are also be tracked to see if the treatment based upon microbiology data is correct. Changes in treatment are assessed to ensure culture data is utilized in treatment and antimicrobial stewardship practices are being followed.

Computing resources. Computational biology work is performed on servers on premise. These servers are secured because they contain clinical data. All HIPAA standards are applied. The server operates on 6× VxRail E560F nodes (PowerEdge R640 1 U rack mount servers) and has dual Intel Xeon Platinum 8260 (24c) 2.4 Ghz with 1,152 GB RAM, 2×1.6 TB SAS SSD cache, 8×7.68 TB SAS SSD capacity, 4×10 Gb data ports, and 1×1 Gb iDRAC management port. This server includes vSphere Enterprise Plus with 3 Years 24×7 Mission Critical Support per node configured to provide the computational infrastructure. The server consists of 288c (691.2 GHz) CPU, and 6.75 TB RAM. Storage estimates reflect 368.64 TB RAW/222 TB usable memory on a RAID6 configuration with 20% vSAN overhead. This server manages all large data sets from RNA sequencing. Because of the depth of sequencing that is needed for RNA splicing analysis (100 million reads vs. 40 million), more data is generated from both sequencing and analysis.

In a preliminary project, the inventors generated one terabyte of sequencing data and another terabyte from the alignment to the genome. Because RNA sequencing data is always identifiable, the data from humans are treated as though it is protected health information (PHI), even though none of the typical identifiers (such as name, date of birth, etc.) are associated with the data.

The following pipeline encompasses the typical analysis: differential expression, RNA analysis is done with Whippet (Sterne-Weiler et al. (2018)). After this, the unmapped reads are analyzed for microbial RNA. The inventors curate a reference genome of all identified species of S. aureus, E. coli, P. aeruginosa, and H. influenza.

Bacterial rearrangements are common across strains. This tool adjusts for rearrangement with a consensus genome to align the un-mapped reads to them. Noureen, Tada, Kawashima, & Arita (2019). This tool allows for visualization and construction of a consensus genome. The conserved and strain specific sequences are kept. Tada, Tanizawa, & Arita (2017). Targets are preferentially chosen from conserved regions. Strain specific targets are used if clinically relevant. Specific resistance genes are also be searched for in the unmapped reads using the STAR aligner.

Polymerase chain reaction (PCR) design. Optimized PCR parameters are essential to ensuring accuracy and reproducibility in qPCR reactions. See Bustin & Huggett (2017); Bustin, Mueller, & Nolan (2020)). Target selection is critical. The preliminary data demonstrate that bacterial reads are measurable from patient with bacteremia and pneumonia and that the reads can be aligned to the organism's genome. RNA sequencing data accumulated from patients with bacteremia or pneumonia due to the specified pathogens are used to identify sequences of interest. These sequences are compared to a pan genome of the same organism to confirm the target is generalizable to the pathogen. Wang et al. (2022). The inventors use Beacon Designer (Premier Biosoft) to design several primer/probe combinations for the sequences, the specificity of which are confirmed by BLAST searches. Primers with low specificity, dimer formation, or that create amplicons with complex secondary structures are excluded. Bustin & Huggett (2017). Primer-BLAST (NCBI) are used as an independent, complementary design strategy; primers identified by both approaches are prioritized. PCR reactions are optimized in the laboratory for temperature and primer concentration for the mastermix. The objective is to establish a standard set of testing conditions.

Testing the PCR. The PCR tests are validated in two ways. First, cDNA libraries used for RNA sequencing are tested. Next, RNA from the blood of patients, both with and without the infection, are used as templates for cDNA synthesis and then PCR. PCRs are applied to the samples from RNA sequencing and an independent cohort of patients to validate the assays. Several primer combinations are evaluated for each target sequence. Bustin & Huggett (2017). SYBR green methodology are used initially to prioritize different primer combinations. Hydrolysis (“Taqman”) probes for qPCR, which were already designed with the primers, is then synthesized for the prioritized primer combinations.

Rigor and reproducibility. RNA is less stable than DNA because of its vulnerability to hydrolysis. This invention uses RNA sequencing and uses methodology to ensure stability. The preliminary data show isolated RNA from critically ill patients and high quality RNA sequencing results. The inventors also focus on isolation methods that are standard and can easily be applied followed so the results can be translated to clinical practice. To enhance robustness during development, it is standard practice for each step of the PCRs (setup, cycling, analysis) to be performed in separate rooms, reducing reactions being contaminated with amplicons from prior runs.

Biological variables. Variables such as age (patients are included across the lifespan, weight, and medical co-morbidities are collected and compared across groups. If these variables, or sex, are significantly different (t test or rank sum), these factors are adjusted for in the analysis via regression.

Cloud based computing. Because of the depth of sequencing that is needed for RNA splicing analysis (100 million reads vs. forty million), more data is generated from both sequencing and analysis (a small study generated one terabyte of sequencing data and another terabyte from the alignment to the genome). With such a large amount of data predicted, the ability to expand and contract the storage space and computing power in the cloud is the ideal choice. This server stores and analyzes data from both mouse and human samples. Because RNA sequencing data is always identifiable, the data from humans are treated as though it is protected health information (PHI), even though none of the typical identifiers (such as name, date of birth, etc.) are associated with the data. The cloud server is only accessible through a hospital virtual desktop and data are saved only to the Azure server or a hospital computer. Data are encrypted while stored, and when in transit to or from the hospital. Any link to typical identifiers (name, date of birth, etc.) are kept separate from the sequencing data. The cloud-based server allows for large data analysis with computing and storage needs changing on a per-use basis. The Azure server is Linux based and uses programming in R and Python. The following pipeline encompasses the typical analysis: differential expression, RNA analysis is done with Whippet. This also includes an entropy measure, and genes of interest undergo gene ontology term analysis. Genes with alternative transcription start and end sites identified through Whippet are correlated with findings from the mountainClimber analysis.

Computational analysis and statistics. RNA sequencing data from the mouse was first checked for quality using FASTQC. RNA-sequencing data collected from the GTEx consortium, and the mouse ARDS model was analyzed with the Whippet software for differential gene processing. Alternative transcription events are those events identified by Whippet as ‘tandem transcription start site,’ ‘tandem alternative polyadenylation site,’ ‘alternative first exon,’ and ‘alternative last exon.’ Alternative RNA splicing events are those events labeled ‘core exon,’ ‘alternative acceptor splice site,’ ‘alternative donor splice site,’ and ‘retained intron.’ Alternative mRNA processing events were determined by a log 2 fold change of greater than 1.5+/−0.2. Statistical significance was calculated by the chi-square p-value of a contingency table based on 1000 simulations of the probability of each result.

Gene ontology (GO) was assessed using The Gene Ontology Resource Knowledgebase. Ashburner et al., Nature Genetics, 25, 25-29 (2000); The Gene Ontology Resource. Nucleic Acids Research, 47, D330-d338 (2019). Genes from the analyses were entered, and outputs were displayed. Outputs from gene ontology do not correlate with actual increase or decrease in a gene's expression but are related to expected based upon the set of genes entered.

Blood sample collection. Blood samples are collected on day 0 of Intensive Care Unit admission. Clinical data including COVID specific therapies was collected prospectively from the electronic medical record and participants were followed until hospital discharge or death. Ordinal scale can be collected as described by Beigel et al., New England Journal of Medicine (2020); along with sepsis and associated sequential organ failure assessment (SOFA) score, and the diagnosis of ARDS. See Singer et al., The Third International Consensus Definitions for Sepsis and Septic Shock (Sepsis-3). JAMA, 315:801-810 (2016); Ferguson et al. The Berlin definition of ARDS. Intensive Care Medicine, 38:1573-1582 (2012).

RNA extraction and sequencing. Whole blood can be collected in PAXgene tubes (QIAGEN, Germantown, MD, USA) and sent to Genewiz (South Plainfield, NJ, USA) for RNA extraction, ribosomal RNA depletion and sequencing. Sequencing can be done on Illumina HiSeq machines to provide 150 base pair, paired end reads. Libraries were prepared to have three samples per lane. Each lane provided 350 million reads ensuring each sample had >100 million reads.

Computational biology and statistical analysis. All computational analysis can be done blinded to the clinical data. The data can be assessed for quality control using FastQC. See Andrews, A quality control tool for high throughput sequence data. FastQC (2014). RNA sequencing data can be aligned to the human genome utilizing the STAR aligner. Dobin et al., Bioinformatics (Oxford, England), 29, 15-21 (2013). Reads that aligned to the human genome can be separated and called ‘mapped’ reads. Reads that do not align to the human genome, which are typically discarded during standard RNA sequencing analysis, were identified as ‘unmapped’ reads. The unmapped reads then align to the relevant comparator and counted per sample using Magic-BLAST. See Boratyn et al., BMC Bioinformatics, 20, 405 (2019). The unmapped reads were further analyzed with Kraken2. See Wood, Lu, & Langmead, Genome Biology, 20, 257 (2019). The analysis used the PlusPFP index to identify other bacterial, fungal, archaeal, and viral pathogens. See Kraken2/Bracken Refseq indexes maintained by BenLangmead, which uses Kutay B. Sezginel's modified version of the minimal GitHub pages theme.

Reads that align to the human genome, the mapped reads, also can undergo analysis for gene expression, alternative RNA splicing, and alternative transcription start/end by Whippet. See Sterne-Weiler et al., Molecular Cell, 72, 187-200.e186 (2018). When comparisons are made between groups (died vs. survived) differential gene expression can be set with thresholds of both p<0.05 and +/−1.5 log 2 fold change. Alternative splicing was defined as core exon, alternative acceptor splice site, alternative donor splice site, retained intron, alternative first exon and alternative last exon. Alternative transcription start/end events can be defined as tandem transcription start site and tandem alternative polyadenylation site. Alternative RNA splicing and alternative transcription start/end events can be compared between groups. See Sterne-Weiler et al., Molecular Cell, 72, 187-200.e186 (2018). Significance was set at great than 2 log 2 fold change as described by Fredericks et al., Intensive Care Medicine (2020). Genes identified from the analysis of mapped reads can be evaluated by GO enrichment analysis (PANTHER Overrepresentation released 20200728). See Mi et al. Nature Protocols, 8, 1551-1566 (2013).

Whippet can generate an entropy value for each gene's identified alternative splicing and transcription event. These entropy values are created with no groups used in the gene expression analysis. To visualize this data, a principal component analysis (PCA) can be conducted to reduce the dataset's dimensionality and obtain an unsupervised overview of trends in entropy values among the samples. Raw entropy values from all samples can be concatenated into one matrix, and missing values were replaced with column means. Mortality can be overlaid onto the PCA plot to assess the ability of these raw entropy values to predict this outcome in this sample set. This analysis was done in R (version 3.6.3).

Kraken2. The following tools are compatible with both Kraken1 and Kraken2. Both tools assist users in analyzing and visualizing Kraken results. Bracken allows users to estimate relative abundances within a specific sample from Kraken2 classification results. Bracken uses a Bayesian model to estimate abundance at any standard taxonomy level, including species/genus-level abundance. Pavian has also been developed as a comprehensive visualization program that can compare Kraken2 classifications across multiple samples. KrakenTools is a suite of scripts to help analyze Kraken results. For more information, persons having ordinary skill in the biomedical art can refer to Wood, Lu, & Langmead, Improved metagenomic analysis with Kraken2, Genome Biology (Nov. 28, 2019).

Assessment of clinical information. RNA sequencing data are interpreted with clinical data collected from the electronic medical record, including endpoints such as mortality, Intensive Care Unit length of stay, hospital length of stay, SOFA score (Shankar-Hari et al. (2016)), ventilator days, renal failure, ARDS (Ferguson et al. (2012)). Culture data are based upon the test results in the microbiology lab and are the gold standard. Clinical responses to antibiotics are also tracked to see if the treatment based upon microbiology data is correct. Changes in treatment are assessed to ensure culture data is utilized in treatment. Antimicrobial stewardship practices are being followed.

Computing resources. All computational biology work is performed on servers on-premises. These servers are secured because they contain clinical data. All HIPAA standards are applied. The server operates on 6× VxRail E560F nodes (PowerEdge R640 1 U rack mount servers) and has dual Intel Xeon Platinum 8260 (24c) 2.4 Ghz with 1,152 GB RAM, 2×1.6 TB SAS SSD cache, 8×7.68 TB SAS SSD capacity, 4×10 Gb data ports, and 1×1 Gb iDRAC management port. This server includes vSphere Enterprise Plus with 3 years of 24×7 mission critical support per node configured to provide the computational infrastructure. The server consists of a 288c (691.2 GHz) CPU and 6.75 TB RAM. Storage estimates reflect 368.64 TB RAW/222 TB usable memory on a RAID6 configuration with 20% vSAN overhead. This server manages all large data sets from RNA sequencing. Because of the depth of sequencing that is needed for RNA splicing analysis (100 million reads vs. 40 million), more data is generated from both sequencing and analysis. In a preliminary project, the inventors generated one terabyte of sequencing data and another terabyte from the alignment to the genome. Because RNA sequencing data is always identifiable, the data from humans are treated as protected health information (PHI), even though none of the standard identifiers (such as name, date of birth, etc.) are associated with the data.

SEQUENCE LISTING

Mutated C110 NAb CDR3 sequence CQQYNNYWAF (SEQ
ID NO: 1).
CDR3 region CQQYNNYWAF (SEQ ID NO: 2).
CDR3 region CQQYNNWPPLTF (SEQ ID NO: 3).
CDR3 region CQQYDNHF (SEQ ID NO: 4).
CDR3 region CQQYNRYWTF (SEQ ID NO: 5).
CDR3 region CQQYNNWPPLFTF (SEQ ID NO: 6).
CDR3 region CQQYNNWPPGFTF (SEQ ID NO: 7).
CDR3 region CQQLNSYPGGTF (SEQ ID NO: 8).
CDR3 region CQQYDSYPWTF (SEQ ID NO: 9).
CDR3 region CQQYNSYSRTF (SEQ ID NO: 10).
CDR3 region CQQYGSFF (SEQ ID NO: 11).
CDR3 region CQQYDKWPFF (SEQ ID NO: 12).
CDR3 region CMQALQTPLTF (SEQ ID NO: 13).
CDR3 region CMQALQTPRTF (SEQ ID NO: 14).
CDR3 region CQQRSNWPPLTF (SEQ ID NO: 15).
CDR3 region CHQYNNW (SEQ ID NO: 16).
CDR3 region CQQFNTWPPE (SEQ ID NO: 17).
CDR3 region CQQYQTWPPLF (SEQ ID NO: 18).
CDR3 region CQQFNNWPPGFSF (SEQ ID NO: 19).
CDR3 region CQQYNTYSQHTF (SEQ ID NO: 20).
CDR3 region CQSYDSSPLF (SEQ ID NO: 21).
CDR3 region CAVWDDSLSGRVF (SEQ ID NO: 22).
CDR3 region CAAWDDSLSGWVF (SEQ ID NO: 23).
CDR3 region CAAWDDSLNGPVF (SEQ ID NO: 24).
CDR3 region CAAWDDSLSGPVF (SEQ ID NO: 25).
CDR3 region CAAWDDNLSGPVF (SEQ ID NO: 26).
CDR3 region CAAWNDSLSGPNWVF (SEQ ID NO: 27).
CDR3 region CATWDDSLNGYAVF (SEQ ID NO: 28).
CDR3 region CAAWDDSLNGHVVF (SEQ ID NO: 29).
CDR3 region CATWDDTLSGLNWVF (SEQ ID NO: 30).
CDR3 region CAGYGDYSDYW (SEQ ID NO: 31).

The following EXAMPLES are provided to illustrate the invention and shall not limit the scope of the invention.

### Example 1

Analysis of Patients with COVID-19

The inventors conducted a prospective analysis of patients with COVID-19 who were admitted to the intensive care unit at a single center. The patients' peripheral blood underwent deep RNA sequencing (>100 million reads) to identify antibodies (Ig heavy, Ig lambda, Ig kappa) created in response to infection. The amino acid sequence for the CDR3 segments of the antibodies were identified.

Samples were collected on day 0 and day 3 of intensive care unit admission. Clinical data were prospectively collected.

The antibodies were characterized and compared to known COVID-19 antibodies using structural methods. Fifteen patients were enrolled with samples from intensive care unit day 0. Twelve patients had samples from intensive care unit day 3. Antibody type and unique CDR3 segment were identified. Patients who survived had significantly more of a type 3 antibody (C135) to SARS-CoV-2 compared to non-survivors (16,315 reads vs. 1,412 reads, p=0.02).

When looking at patients across time points, there appears to be a critical intervention point in patients with reduced levels of this antibody.

Results. Study population, participant characteristics, and RNA sequencing. A total of fifteen patients had samples collected on day 0 and of those fifteen, twelve had samples collected on day 3. Two patients who did not provide a second time point were discharged from the intensive care unit and one died prior to the second time point.

TABLE 5 shows demographic data and antibody types per group (survivor vs. non-survivor) at ICU day 0 and ICU day 3.

TABLE 5
Clinical and demographic data

| | Day 0 | |
|---|---|---|
| Demographics and Antibody Types | Survivor (8) | Non-Survivor (7) |
| Median age (SD) | 53.9 (22.7) | 64.1(13.8) |
| Female - no. (%) | 2 (25) | 2 (29) |
| Race | | |
| White - no. (%) | 2 (25) | 4 (57) |
| Black or African American - no. (%) | 1 (13) | 1 (14) |
| Other Race - no. (%) | 5 (62) | 2 (29) |
| Ethnicity | | |
| Hispanic - no. (%) | 4 (50) | 5 (71) |
| Median BMI (SD) | 28.1 (4.1) | 31.5 (4.9) |
| Ig Heavy (SD) | 18,77.6 (31,136.3) | 17,247 (17,780) |
| Ig Kappa (SD) | 70,548.6 (136,600.2) | 43,630.7 (44,591.6) |
| Ig Lambda (SD) | 9,289.9 (11,302.4) | 19,000.9 (24,621.1) |
| Total Reads (SD) | 99,478.5 (178,615) | 80,942.6 (86,484.6) |
| | Day 3 | |
| Demographics and Antibody Types | Survivor (6) | Non-Survivor (6) |
| Median age (SD) | 53.5 (18.5) | 60.9 (11.8) |
| Female - no. (%) | 2 (33) | 2 (33) |
| Race | | |
| White - no. (%) | 1 (17) | 3 (50) |
| Black or African American - no. (%) | 1 (17) | 1 (17) |
| Other Race - no. (%) | 4 (66) | 2 (33) |
| Ethnicity | | |
| Hispanic - no. (%) | 3 (50) | 4 (66) |
| Median BMI (SD) | 26.6 (3.6) | 31.7 (5.4) |
| Ig Heavy (SD) | 45,029.8 (48,919.6) | 39,982.1 (33,397.3) |
| Ig Kappa (SD) | 65,278.3 (83,406) | 34,784.3 (22,616.4) |
| Ig Lambda (SD) | 31,087.3 (29,572.1) | 36,133 (30,801) |
| Total Reads (SD) | 142,306.8 (141,072.9) | 111,939.2 (84,022.7) |

The results showed no differences between the numbers of counts for Ig Heavy, Ig kappa, and Ig lambda between survivors and non-survivors on days 0, 3, or in aggregate (TABLE 5). When comparing across time points, there was a significant increase Ig lamda across all patients (16,590 vs 33,610, p=0.009). When looking at survivors alone, this difference was present (11,313 vs 31,087, p=0.0426) but not in those who died (21,868 vs 36,133, p=0.1504).

All CDR3 sequences identified in each patient with associated counts and categorization for antibody type, COVID-19 antibody class, and structure of COVID-19 antibody are present for day 0 (see TABLE A2) and day 3 (see TABLE A3).

When comparing classes of COVID-19 antibodies (Class 1, 2, 3, 4) there is no difference between survivors and non-survivors or across time points. However, when assessing the number of counts that align to the C135 antibody, survivors had significantly more (15,059.4) than non-survivors (1,412.7, p=0.016; FIG. 1) on day 0 but not on day 3. All other distinct COVID-19 antibodies described in TABLE A1 had no difference across survivors vs. non-survivors or across time points.

Antibodies identified as unique to survivors and non-survivors on day 0 are presented in TABLE 6. See the full list TABLE A4 for survivors and TABLE A5 for non-survivors. Among survivors the most antibodies categorize as Class 3. Among non-survivors many of the top antibodies were categorized as Class 4 with other classes of antibodies having fewer reads.

The Class 3 sequence of light chain CDR3 from the most frequent RNAseq reads exclusive to surviving patients was modeled into a Cryo-EM structure of the C135 antibody bound to the SARS-CoV-2 spike protein (FIG. 2A, PDB: 7K8Z). This model shows the strong intermolecular forces between the light chain CDR3 and the RBD epitope. The antibody bound structure was then aligned to a structure of the SARS-CoV-2 spike protein bound to the extracellular domain of ACE2 (FIG. 2B, PDB: 6M0J). The latter model highlights Class 3 CDR3 binding at a location distinct from ACE2. This contrasts Class 2 antibodies commonly detected in non-survivors, which share a binding site with the ACE2 receptor on the SARS-CoV-2 spike RBD.

Conclusions. Deep RNA sequencing is a tool to identify antibodies produced in response to COVID-19 infections. Typical identification of antibodies is time consuming. Shrestha, Tedla, & Bull, Frontiers in Immunology, 12, 752003 (2021). Antibodies identified from plasma do not provide as much detail as is gleaned from the RNA sequencing data, such as Ig classes and the actual amino acid sequence of the antibody. By identifying antibodies that are unique to survivors, persons having ordinary skill in the biomedical art can identify the CDR3 sequences most likely to positively impact infection. Combining the amino acid sequence and structural biology techniques, persons having ordinary skill in the biomedical art can identify the binding locations for these antibodies created in vivo in a critically ill patient.

Most previous works assessing immunoglobulin light chain types of lambda and kappa were limited to hematologic malignancies and HIV infection. Andrei & Wang, Hematology/Oncology and Stem Cell Therapy 12, 71-81 (2019); Müller & Köhler, International Reviews of Immunology, 14, 339-49 (1997); & Terrier et al., Autoimmunity Reviews, 13, 319-26 (2014). The impact of an increase of Ig lambda at ICU day 3 in survivors is unknown. This increase can be used as a marker of potential recovery. The use of this technology could enhance the study of class types in antibodies not only during infectious disease but also enhancing the previous work in hematologic malignancies and possible transplant medicine.

All four classes of antibodies (TABLE A1) bind to the SARS-CoV-2 spike RBD with strong interactions between the light chain CDR3 and the respective epitopes. Barnes et al., Nature, 588, 682-7 (2020). Class 1 and 2 antibodies bind to an epitope that causes steric clash with the ACE2 binding site. Antibodies that bind to the ACE2 motif on the RBD are known to be influenced by mutations in the virus. Starr et al., Nature (2021). The proliferation of variants continues as the pandemic continues. Drews et al., Transfusion (2021). It is important to ensure that antibodies exist that are not prone to mutations at the ACE2 motif. Class 3 and 4 antibodies bind to an epitope orthogonal to the ACE2 binding site. Non-ACE2 competitive antibodies (such as class 3 and 4) increase attenuation of the virus in cells that overexpress ACE2.4 ACE inhibitors are known to increase ACE2 expression. Vaduganathan et al., The New England Journal of Medicine, 382, 1653-9 (2020). There was no definitive correlation with outcome. Reynolds et al., The New England Journal of Medicine, 382, 2441-8 (2020). This lack of impact of ACE inhibitors on outcome may be due to the class of antibodies that a patient creates in response to infection. This sample size is not sufficient to assess the impact of pre-infection ACE inhibitor use but binding to the spike protein away from ACE2 binding site (FIG. 2) appears to be beneficial and supports why having more C135 (Class 3) antibodies occurs in survivors (FIG. 1).

The spike protein forms a trimmer where all must be in the “up” position for RBD binding to ACE2. Lobo & Warwicker, Computational and Structural Biotechnology Journal, 19, 5140-8 (2021). Simulations of spike trimers showed its transition between up, down, and locked (stabilized down) acidic pH confers stability to the locked structure. Lobo & Warwicker, Computational and Structural Biotechnology Journal, 19, 5140-8 (2021). Class 2 and 3 antibodies can bind the spike protein in both the up and down confirmations. Class 1 and 4 can only bind in the up confirmation. Binding spike in both the up and down position can prevent cell fusion. Starr et al., Nature (2021). Among antibodies exclusive to survivors, on ICU day 0, they had many more class 3 antibodies while antibodies exclusive to non-survivors on day 0 aligned more frequently to class 4. See TABLE 6, TABLE A4, and TABLE A5. Class 3 antibodies can bind separate from the ACE2 epitope and are able to bind in both up and down orientation. pH influences the up/down confirmation. Patients who are critically ill are typically acidotic. Hence, an antibody that binds in both confirmations is useful for the ICU patient.

Some engineered antibodies appear to use sites that cross class 1 and class 4 antibodies. Rappazzo et al., Science, 371, 823-9 (2021). The widely reported Regeneron Antibody is of class 3, but distinct from C135. Barnes et al., Nature, 588, 682-7 (2020). C135 increased in survivors compared to non-survivors on ICU day 0. In one patient with a low level of C135 compared to other survivors, an increase in another class 3 antibody was like REGN10987 (TABLE A2 and TABLE A3). If patients with low levels of class 3 antibodies, specifically C135, are identified by deep RNA sequencing on ICU day 0, the patients could be treated with commercially available antibodies.

The immune system unleashes a volley of antibodies when confronted with a pathogen such as COVID-19. Mutant spike protein from SARS-CoV-2 is resistant to sera from convalescent or mRNA vaccine recipients but not to patients infected and then vaccinated. Schmidt et al., Nature (2021). This response shows the importance of both antibody development and vaccination to care for COVID-19. Deep RNA sequencing with analysis for CDR3 sequences could find novel antibodies in patients who are hospitalized with future variants. Deep RNA sequencing could also assess response to vaccines, especially in patients with immune suppression. Yahav et al., BMJ Open, 11, e055611 (2021). A cocktail of antibodies from TABLE 6 may be beneficial in treating COVID-19 in addition to ubiquitous vaccination.

TABLE 6 shows the top twenty CDR3 Alignments that were exclusive to survivors and non-survivors on ICU day 0. The alignment is the common sequence to which several CDR3 from each patient aligned (Cluster Counts). The CDR3 counts represent the number of times a CDR3 mapped from the survivors or non-survivors. The class and ID are defined. See Barnes et al., Nature, 588, 682-7 (2020) and references in TABLE A1.

TABLE 6A
CDR3 Alignments Exclusive to Survivors Day 0

| Alignment | Cluster Counts | CDR3 Counts | Class | ID |
|---|---|---|---|---|
| CQQYNNYWAF (SEQ ID | 43 | 75616 | C3 | C135 |
| NO: 2) | | | | |
| CQQYNNWPPLTF (SEQ | 52 | 60021 | C3 | REGN10987 |
| ID NO: 3) | | | | |
| CQQYDNHF (SEQ ID | 42 | 48510 | C3 | REGN10987 |
| NO: 4) | | | | |
| CQQYNRYWTF (SEQ ID | 26 | 44085 | C3 | C135 |
| NO: 5) | | | | |
| CQQYNNWPPLFTF | 31 | 36103 | C3 | REGN10987 |
| (SEQ ID NO: 6) | | | | |
| CQQYNNWPPGFTF | 35 | 35532 | C3 | REGN10987 |
| (SEQ ID NO: 7) | | | | |
| CQQLNSYPGGTF (SEQ | 26 | 35395 | C1 | B38 |
| ID NO: 8) | | | | |
| CQQYDSYPWTF (SEQ | 26 | 34909 | C3 | C135 |
| ID NO: 9) | | | | |
| CQQYNSYSRTF (SEQ | 32 | 34521 | C3 | C135 |
| ID NO: 10) | | | | |
| CQQYGSFF (SEQ ID | 25 | 33616 | C1 | C102 |
| NO: 11) | | | | |
| CQQYDKWPFF (SEQ ID | 25 | 30823 | C4 | S2A4 |
| NO: 12) | | | | |
| CMQALQTPLTF (SEQ ID | 18 | 26607 | C2 | C002 |
| NO: 13) | | | | |
| CMQALQTPRTF (SEQ | 17 | 26599 | C2 | C104 |
| ID NO: 14) | | | | |
| CQQRSNWPPLTF (SEQ | 39 | 20622 | C3 | REGN10987 |
| ID NO: 15) | | | | |
| CHQYNNW (SEQ ID NO: | 30 | 19583 | C3 | REGN10987 |
| 16) | | | | |
| CQQFNTWPPE (SEQ ID | 17 | 19345 | C1 | B38 |
| NO: 17) | | | | |
| CQQYQTWPPLF (SEQ | 28 | 17810 | C1 | B38 |
| ID NO: 18) | | | | |
| CQQFNNWPPGFSF | 17 | 16145 | C1 | B38 |
| (SEQ ID NO: 19) | | | | |
| CQSVDSSGIYI (SEQ ID | 27 | 15458 | C4 | S2A4 |
| NO: 32) | | | | |
| CQSADSSGTYPDVVF | 33 | 15132 | C4 | S2A4 |
| (SEQ ID NO: 33) | | | | |

TABLE 6b
CDR3 Alignments Exclusive to Non-Survivors Day 0

| Alignment | Cluster Counts | CDR3 Counts | Class | ID |
|---|---|---|---|---|
| CQQSYSTPLFAF (SEQ | 28 | 140569 | C4 | EY6A |
| ID NO: 34) | | | | |
| CQQYYTTPMYAF (SEQ | 23 | 125715 | C4 | CR3022 |
| ID NO: 35) | | | | |
| CQQYYSTPPSF (SEQ ID | 34 | 110321 | C4 | CR3022 |
| NO: 36) | | | | |
| CQSADSSGTWVF (SEQ | 37 | 62077 | C4 | S2A4 |
| ID NO:37) | | | | |
| CQSADTSGTYGNWVF | 22 | 61809 | C3 | S309 |
| (SEQ ID NO: 38) | | | | |
| CQSADTSGPYRDVVF | 17 | 61626 | C3 | S309 |
| (SEQ ID NO: 39) | | | | |
| CQSPDSSATYQV (SEQ | 11 | 61577 | C4 | S2A4 |
| ID NO: 40) | | | | |
| CQSADNSGSYGIF (SEQ | 22 | 51193 | C4 | S2A4 |
| ID NO:41) | | | | |
| CQQYGSSVTF (SEQ ID | 27 | 48316 | C1 | C102 |
| NO: 42) | | | | |
| CQSADSSGTYKMLF | 16 | 30329 | C4 | S2A4 |
| (SEQ ID NO: 43) | | | | |
| CQRYNNYPYIF (SEQ ID | 15 | 25022 | C3 | C110 |
| NO: 44) | | | | |
| CQRYNTAPYTF (SEQ ID | 12 | 22628 | C3 | C110 |
| NO: 45) | | | | |
| CQQVYDTPRTF (SEQ ID | 12 | 19935 | C2 | C002 |
| NO: 46) | | | | |
| CMQGTHWPPIIF (SEQ ID | 19 | 14254 | C1 | B38 |
| NO: 47) | | | | |
| CIQGTHWPPSAF (SEQ | 14 | 14235 | C1 | B38 |
| ID NO: 48) | | | | |
| CMQGTHWPPSTF (SEQ | 16 | 14233 | C1 | B38 |
| ID NO: 49) | | | | |
| CMQGTHWPPAF (SEQ | 20 | 14141 | C1 | B38 |
| ID NO: 50) | | | | |
| CMQGTLWPPSF (SEQ ID | 13 | 14091 | C1 | B38 |
| NO: 51) | | | | |
| CMQGTHWPPGLTF | 13 | 14077 | C1 | B38 |
| (SEQ ID NO: 52) | | | | |
| CMQGTHWPPSFTF (SEQ | 14 | 14076 | C1 | B38 |
| ID NO: 53) | | | | |

### Example 2

Design a Direct from Blood, without the Need for Culture, Reverse Transcriptase Polymerase Chain Reaction (RT-qPCR) Test for Bacteria Causing Bacteremia, Specifically S. aureus, E. coli, P. aeruginosa, Based on the RNA Identified in Patients with Bacteremia Caused by these Organisms (A1a)

Rationale. Blood cultures are the current gold standard for pathogen diagnostics but take days. Blood cultures have a known contaminant rate, which can adversely affect treatment and disease progression, as shown in the COVID pandemic. Yu et al. (2020). RNA sequencing is an emerging technology that can enhance the diagnostic capabilities. Unmapped reads, i.e., reads that do not align to the human genome, are typically discarded in RNA sequencing data from humans. When the depth of the RNA sequencing is sufficient, these unmapped reads can provide useful clinical information. The unmapped reads found in the blood of patients with bacteremia are used to inform the development of a diagnostic PCR.

The gene expression of the bacteria discriminates between infection and simply colonization. D'Mello et al. (2020). Targeting RNA is more specific than DNA by eliminating the signals of free DNA from dead bacteria or pathogen DNA released from immune cells combating the infection. Opota, Jaton, & Greub (2015).

TABLE 7
Creation of Bacterial Genomes

| Bacteria | Genomes | Plasmids |
|---|---|---|
| S. aureus | https://www.ncbi.nlm.nih.gov/genome/154 | 958 |
| E. coli | https://www.ncbi.nlm.nih.gov/genome/167 | 5895 |
| P. aeruginosa | https://www.ncbi.nlm.nih.gov/genome/187 | 155 |
| H. influenza | https://www.ncbi.nlm.nih.gov/genome/165 | 1 |

Assay 1. Assess the RNA sequencing data from patients with blood infections due to S. aureus, E. coli, and P. aeruginosa. Unmapped reads or reads that do not align to the organism of interest, are typically discarded. These reads are used to identify bacterial RNA in the blood. This was initially done using Kraken2. Wood, Lu, & Langmead (2019). For more granularity, the inventors assembled custom genomes to which the unmapped reads are aligned using STAR RNA-sequencing aligner. Dobin et al. (2013). These genomes are based upon the common genome in TABLE 7 but also include sequences from other chromosomes and the plasmids attributed to those bacteria, creating a pan-genome. Eizenga et al. (2020). Samples from patients with S. aureus are used to identify the significant reads that align to this bacterium and repeated for other pathogens of interest. This gives a total read count for each bacterium and the portions of the genome with the most abundant reads. From these abundant reads, PCR primers are designed to test for the pathogens based upon large reads of common areas across many patients.

Assay 2. Create RT-qPCR primers to identify S. aureus, E. coli, and P. aeruginosa causing bacteremia. Using the targets of interest from the deep RNA sequencing data, PCR primers are designed to cover these parts of the bacterial genome identified. It may be necessary to have multiple primers for multiple targets to identify one pathogen, however this are accomplished through multiplexing that is possible with the NeuMoDx instrument from the industry partner. The target of these primers are the RNA in the blood, a reverse transcriptase reaction are used to create the cDNA for the PCR.

Expected results. The preliminary data show that patients with bacteremia have bacterial RNA in their blood that correlates with the causative organism. The inventors anticipate there are a set of highly expressed genes from each of the bacteria during infection that can be the basis for identification. The inventors expect genes like the coagulase gene to be detected in patients with Staphylococcus aureus bacteremia. Cheng et al. (2010). The inventors prioritize PCR targets unique to the bacteria being tested and distinct from other bacteria. Additional findings include observations that gene expression of the bacteria can also determine colonization versus infection based on expression pattern and abundance. D'Mello et al. (2020). The number of reads (i.e., transcript abundance) may correlate with patient condition or patient outcomes. Abundant clinical data is associated with patients from with the samples are derived. Read frequency or abundance on RT-qPCR are evaluated for these correlations.

Potential pitfalls and alternatives. Maybe the bacteria that are identified in the sequencing cannot be correlated to microbiology culture. This could be due to blood cultures being negative in 50% of blood stream infections, likely due to low numbers of bacteria in the blood or the impact of antibiotics before the sample is obtained. Opota, Jaton, & Greub (2015). Blood culture could identify the wrong pathogen while another pathogen could cause infection, i.e., a contaminant. the approach includes aligning unmapped reads using Kraken2 to identify background levels of sequences from unrelated bacteria that could be commensals or contaminants. A single gene may not uniquely identify an organism, reducing specificity of the test. In that situation, the inventors test gene combinations as described above. Alternatively, unique alleles/SNPs are used to define a specific pathogen. Established techniques are used to measure SNPs in the RT-qPCR format.

### Example 3

Validate RT-PCR Tests in Samples from Patients with and without Bacteremia (A1b)

Rationale. After targets for identification of the pathogens are determined, it is imperative to ensure that these targets can be used clinically. RT-qPCR allow for the identification of pathogens, directly from blood, without culture, in less than four hours. RT-qPCR also allow for faster, pathogen-directed antibiotic selection. Blood culture collection is recommended before antibiotic administration to enhance the diagnostic sensitivity of the blood culture. See Evans et al. (2021). With this diagnostic test proposed here, antibiotics are not expected to influence the RNA present at the blood draw.

Assay 1. Test PCR primers on samples used for RNA sequencing. The cDNA libraries created for RNA sequencing are accessed as the initial test of the PCR primers. Because the RNA sequencing data determined these RNA segments were present, this are the first step in assessment of the utility of these novel PCR tests for the bacteria. The cDNA from all samples with positive cultures for each of the bacteria are used for this assay. Each cDNA sample are tested using the PCR primers for all pathogens. As a negative control for specificity, the inventors also cDNA from the blood of patients and normal controls that have no infections.

Assay 2. Validate PCR primers on samples that mimic collection for clinical use. To obtain sensitivity and specificity in line with FDA requirements, samples from patients with and without confirmed blood infections due to the pathogens of interest are identified from banked clinical specimens, including PAXgene tubes. The PAXgene tubes are being collected at the time of blood culture collection and stored. In the experience, PAXgene tubes completely stabilize high quality RNA. To enhance robustness of the testing, these tubes are blinded to the team performing the PCR assays. The RNA are extracted and globin and rRNA are reduced using commercially available kits. cDNA libraries are made with reverse transcriptase and then PCR are done with the primers. PAXgene tubes are used but the RT-qPCR are done immediately to ensure the result returns in less than four hours.

Expected results. A panel of PCR primers are developed and optimized on a machine that can be easily translated to a clinical microbiology laboratory. The tests identify S. aureus, E. coli, and P. aeruginosa directly from the blood through extraction of RNA, reduction of globin and rRNA, and creation of cDNA for the PCR. These tests have sensitivity and specificity in line with requirements of the FDA. These direct from blood PCR are initially done for S. aureus, E. coli, and P. aeruginosa. Through collecting samples from patients in PAXgene tubes with other infections, the PCR panel can be expanded as new targets from additional pathogens are identified. Because this PCR is rapid, it could be used to monitor treatment impact, a practice not currently done as culture takes days to return. If successful treatment is detected, antibiotic course could be shortened and therefore enhance antimicrobial stewardship.

Potential pitfalls and alternatives. Blood is known to interfere with PCR when identifying DNA, but not RNA. Sidstedt et al. (2018). The number of reads may need to be a minimal amount to be translatable to detection by PCR. Deep RNA sequencing may find a gene that identifies infection, but PCR conditions cannot be optimized to replicate the finding. This problem can be solved when RNA sequencing costs and time are reduced. RNA sequencing should take less than four hours at a depth of 100 million reads or more.

### Example 4

Design a Direct from Blood, without the Need for Culture, Reverse Transcriptase Polymerase Chain Reaction (RT-PCR) Test for Bacteria Causing Pneumonia, Specifically S. aureus, P. aeruginosa, H. influenzae, Based on the RNA Identified in Patients with Pneumonia Caused by these Organisms (A2a).

Rationale. The diagnosis of hospital-acquired pneumonia is complex. Modi & Kovacs (2020). Bronchial alveolar lavage (BAL) is the gold standard, similar to blood culture for bacteremia. Because BAL requires an invasive intervention (bronchoscopy) that can worsen the clinical picture, screening tools are used to decide when to perform them, hence the yield is higher than blood culture. A direct from blood test that provides the same diagnosis obviates the need for the invasive bronchoscopy intervention. The assays described below parallel those in EXAMPLE 2 with an independent cohort of patients diagnosed with pneumonia and who undergo BAL.

Assay 1. Assess the RNA sequencing data from patients with pneumonia due to S. aureus, P. aeruginosa, and H. influenzae. As described in the EXAMPLE above for the blood infections, unmapped reads are aligned to genomes of interest to identify genes with increased expression in patients with infection diagnosed by BAL. The blood are collected in PAXgene tubes at the time of BAL. S. aureus and P. aeruginosa genes that are identified are compared to the genes identified for bacteremia. From these reads, PCR primers are developed.

Assay 2. Create RT-PCR primers to identify S. aureus, P. aeruginosa, and H. influenzae causing pneumonia. Using the reads generated from Assay 1, PCR primers are developed to identify pathogens causing hospital acquired pneumonia and applied to the sequenced samples and an independent cohort.

Expected results. The preliminary data show that patients in the ICU have bacterial RNA in the blood. There are a set of highly expressed genes from the bacteria during infection that can be used as the basis for identification. One outcome is that these genes differ from the genes expressed during bacteremia because some suggested that gene expression changes from the bacteria based on site of infection/colonization. The inventors have RNA sequencing data from patients with bacteremia and pneumonia due to similar pathogens and can see if different genes are expressed at higher rates. Primers can be developed for each pathogen based upon site of infection to guide diagnosis. An alternative outcome is that the same target sequences are found in bacteremia and pneumonia. This would simplify product development on the NeuMoDx and require the test be integrated into other clinical diagnostics such as X-rays.

Potential pitfalls and alternatives. The technical approach is similar to EXAMPLE 2, which the inventors have shown is feasible. Because the infection is in the lung, there may be no bacterial RNA identified in the blood of these patients, but other studies dispute this possibility. D'Mello et al. (2020). Bacterial DNA was detected in the blood of pneumonia patients. Langelier et al. (2020). The lung has a large surface area for gas exchange that would facilitate transfer of stable RNA or RNA in micro vesicles from the infection into the bloodstream. Blenkiron et al. (2016). Bacteremia complicates pneumonia in 6-17% of cases, depending on severity. Zhang, Yang, & Makam (2019). A subset of the pneumonia patients are expected to have target sequences shared with the patients studied in EXAMPLE 1. If the sequencing analysis cannot distinguish between the subgroups of pneumonia patients with and without bacteremia, clinical data are used to guide management.

### Example 5

Validate RT-qPCR Tests in Samples from Patients with and without Pneumonia (A2b).

Rationale. The PCR targets identified by sequencing can be used clinically. Hospital acquired pneumonia typically rapidly deteriorates a patient, so faster diagnosis is essential. RT-qPCR allows for the identification of pathogens, directly from blood, without culture, in less than four hours, and faster selection of pathogen-directed antibiotics. The goal is to eliminate invasive bronchoscopies, which can delay antibiotic administration and increase risks to the patient.

Assay 1. Test PCR primers on samples used for RNA sequencing. RNA sequencing cDNA libraries again are the initial test of the PCR primers. The cDNA from all samples with positive BAL cultures for each of the bacteria are used for this assay. Each cDNA sample are tested using the PCR primers for all pathogens. The inventors also use cDNA from patients that had no hospital-acquired pneumonia.

Assay 2. Test PCR primers on samples that mimic collection for clinical use. This test is done obtain sensitivity and specificity in line with FDA requirements. PAXgene tubes from patients with and without confirmed hospital acquired pneumonia due to the pathogens of interest are identified. The PAXgene tubes are collected at the time BAL collection. The patients' infection status are blinded to the researchers performing the PCRs. The stabilized RNA are extracted from the PAXgene tubes and globin and human rRNA are depleted using a commercial kit from New England Biolabs. cDNA are made with reverse transcriptase and then PCR are done with the primers. PAXgene tubes are used. RT-PCR are done immediately to ensure result in less than four hours.

Expected results. Specific RT-qPCR assays validate the sequencing and diagnose hospital-acquired pneumonia due to S. aureus, P. aeruginosa, and H. influenzae from a direct from blood sample in fewer than four hours. Target abundance vary among patients (see, e.g., TABLE 1), which are correlated with severity of the pneumonia. Efforts are directed to finding primers that diagnose pneumonia and that are distinct from those for bacteremia despite being due to the same pathogen.

### Example 6

Using the RNA from Patients with Infections, Design an RT-qPCR for the Most Common Resistance Genes Expressed that would Influence Treatment for S. aureus, E. coli, P. aeruginosa, and H. influenzae (A3a).

Rationale. While identifying the causative pathogen faster aids antibiotic selection, knowledge of antimicrobial resistance is critical to managing patients with serious infections. Delays in antibiotics worsen outcomes for all patients, including those with resistant organisms. Bonine et al. (2019). Overtreatment of organisms that do not carry resistance determinants also worsens outcomes. Rhee et al. (2020). The objective of this EXAMPLE is to harness data from RNA sequencing to inform PCR-based diagnostics of antimicrobial resistance that are clinically relevant. Reads from the sequencing studies described above are aligned to a “genome” of resistance genes of interest, then novel PCR primers are created to test against clinical specimens. These are “phenotypic” measurements of antibiotic resistance because gene expression and resistance phenotypes are closely linked. Suzuki, Horinouchi, & Furusawa (2014).

Assay 1. Assess the RNA sequencing data from patients with infections due to S. aureus, E. coli, and P. aeruginosa, and H. influenza for resistance genes. A “genome” are made using clinically relevant resistance genes. For Staphylococcus aureus, the inventors include mecA (methicillin resistance) (Chambers & Deleo (2009); Guo et al. (2020)), qacA, norA, smr (efflux transporters of quinolones and tetracyclines) Guo et al. (2020), beta-lactamase (hydrolyses cefazolin) (Guo et al. (2020)), and VRSA (vanA, vanB, vanC, vanX, vanY, vanA). Escherichia coli targets include multiple beta-lactamases: basic beta-lactamases cleaving ampicillin, ESBL genes (TEM-1, TEM-2, and SHV-1) CTX-M (see TABLE 1), ampC, carbapenemases: KPC (class A), metallo (class B: IMP, VIM, NDM-1), OXA (class D) (Bajaj, Singh, & Virdi (2016), GyrA and ParC (fluoroquinolone resistance) (Tchesnokova et al. (2019)), acrB (Karczmarczyk et al., 2011), ompF, Efflux pumps PabetaN, and qnr (Salah et al. (2019)). For Pseudomonas aeruginosa ampC, oprM, mexY (efflux transporters for quinolones and aminoglycosides) (Islam et al. (2009)), bla, gyrA, gyrB, parC (for quinolones) (Yang et al. (2015)), and aac(6′)-lb,aphA1, and aadB (aminoglycosides) (Teixeira et al. (2016)). For Haemophilus influenzae TEM-1 and ROB-1 (Gutmann, Williamson, Collatz, & Acar (1988); Tristram, Jacobs, & Appelbaum (2007)). (52, 53) Using these genes, PCR primers are identified based upon RNA data from patients with these infections. This again are a primer for RT-PCR as the target are RNA. Using RNA as the target yields better results rather than DNA. This tool adapted for use with RNA data could enhance the phenotypic correlation using this data set. Bortolaia et al. (2020).

Assay 2. Create RT-PCR primers to identify resistance genes. Using the targets of interest from the deep RNA sequencing data, PCR primers are designed to cover resistance genes. Multiple primers may be needed to identify resistance genes for one pathogen. This are accomplished through multiplexing that is possible with the machine from the industry partner. The target of these primers are the RNA in the blood, a reverse transcriptase are used to create the cDNA for which the primers interact. Targeting RNA has a better phenotypic correlation than targeting DNA from the pathogen because RNA signifies that the gene is being actively expressed.

TABLE 8
Antibiotic resistance markers and therapeutic considerations.

| | Resistance | Antibiotic if | Antibiotic if | |
|---|---|---|---|---|
| Bacteria | Gene | present | absent | Ref. |
| S. aureus | mecA | vancomycin | nafcillin | Chambers (1997) |
| | blaZ | nafcillin | cefazolin | Dingle et al. (2022) |
| | VRSA (vanA) | daptomycin | vancomycin | |
| E. coli | ESBL | carbapenem or | ampicillin or | Paterson et al. (2001); |
| | | ceftolozane-tazo | 3GenCeph | Rupp & Fey (2003) |
| | Class A | ceftazidime-avi | ampicillin or | Rivera-Izquierdo et al. |
| | carbapenemase; | or aztreonam or | 3GenCeph | (2021) |
| | Class B | tigecycline | | |
| | carbapenemase; | | | |
| | Class D | | | |
| | carbapenemase | | | |
| | gyrA, parC | ampicillin or | fluoroquinolone | Tchesnokova et al. |
| | | cephalosporin | | (2019) |
| P. | ampC | carbapenem | cefepime/ | Jacoby (2009) |
| aeruginosa | | | ceftazidime | |
| | oprM, mexY | non- | fluoroquinolone | Yang et al. (2015) |
| | | fluoroquinolone | | |
| | gyrA/parC point | non- | fluoroquinolone | Yang et al. (2015) |
| | mutation | fluoroquinolone | | |
| H. | tem-1, rob-1 | amoxicillin-clav | amoxicillin | Gutmann, Williamson, |
| influenza | | | | Collatz, & Acar (1988); |
| | | | | Tristram, Jacobs, & |
| | | | | Appelbaum (2007) |

(tazo, tazobactam; avi, avibactam; 3GenCeph, 3^{rd} generation cephalosporin; clav, clavulanic acid.)

Potential pitfalls and alternatives. In one detailed study of transcription and protein abundance in Escherichia coli, there was a lack of correlation between RNA and protein levels. Taniguchi et al. (2010). Though the overall abundances were not correlated, enzyme transcription and translation were closely correlated. Taniguchi et al. (2010). Some resistance phenotypes, such as fluoroquinolone resistance due to gyrA, gyrB, and parC, are mediated by SNPs. The PCR primers are adapted for SNP detection such as the TaqMan assay. Easterday, Van Ert, Zanecki, & Keim (2005). For some resistance mechanisms, such as beta-lactamases, there are too many individual genes to test. In this situation, the inventors use k-mer analysis to identify primers capable of detecting entire classes of beta-lactamases. Marini et al. (2022). Ultimately, there are concerns for whether RNA-based detection of resistance is sufficiently comprehensive to be used in clinical practice. Regulatory RNA may play a role in resistance but not be detected by the sequencing approach. Dersch, Khan, Mühlen, & Görke (2017). In this situation, the inventors evaluate more patient specimens and alter sequencing protocols to detect unconventional RNAs.

### Example 7

Validate PCR Tests for Resistance Genes in Samples from Patients with and without Infections (A3b).

Rationale. Although the resistance genes can be identified with deep RNA sequencing, fast identification with PCR is necessary to be clinically relevant. If the genes are identified, treatment can be changed (TABLE 8).

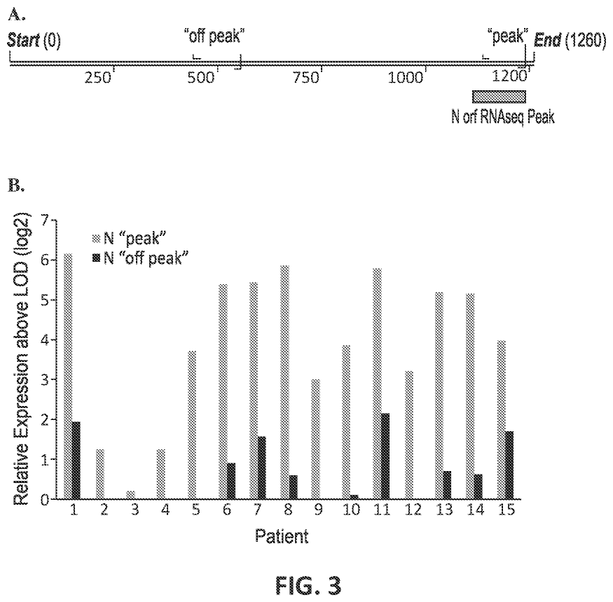

Assay 1. Test PCR primers on samples used for RNA sequencing. cDNA libraries used for RNA sequencing are the initial test of the PCR primers. See FIG. 3. The cDNA from all samples with positive cultures with resistance are used as the positives. Each cDNA sample are tested using the PCR primers for all resistance genes to assess primer specificity.

Assay 2. Test PCR primers on samples that mimic collection for clinical use. These tests are done obtain sensitivity and specificity in line with FDA requirements. PAXgene tubes from patients with and without confirmed infections with resistance are used as essential negative controls. The assays include a positive control gene like actin to confirm PCR reaction in each specimen.

Expected results. Sets of PCR primers identified in this EXAMPLE detect resistance in these validation studies. The most straightforward tests are for the presence or absence of RNA encoding a resistance mechanism, such as mecA in MRSA. Although a molecular test is used in the clinical microbiology lab to diagnose MRSA, this test requires a positive blood culture bottle. The objective is to validate an RNA-based blood test that alters treatment described in TABLE 8. That returns results in less than four hours without having to culture the patient's blood.

Potential pitfalls and alternatives. The principal concerns are for the level of target sequence found in blood, i.e., sensitivity, and the ability to identify primers that amplify the expected sequence, i.e., specificity. Strategies for improving sensitivity include using more cDNA in the PCR reaction and conducting a nested PCR. The inventors have not encountered evidence for inhibition of PCR reactions, which is due to the additional processing involved with using RNA as a PCR template. The inventors also continue to use a positive reference gene, such as actin, to test for PCR inhibitors. There may be a large number of potential sequences that could convey a phenotype, such as the large family of beta-lactamases. k-mer analysis are used to identify sequences that represent the family, and primers are designed against that analysis. Modified PCR reactions, such as TaqMAMA, are used when mutations of pre-existing genes convey a resistant phenotype, as SNPs in gyrA and parC that are responsible for fluoroquinolone resistance. Another possibility is that important resistance mechanisms are infrequently encountered in the patient population, such as carbapenemase production. To create a more comprehensive test under those circumstances, the inventors can evaluate appropriate resistant strains in vitro, such as from the CDC & FDA Antibiotic Resistance Isolate Bank that is available to researchers. It may also be necessary to expand sample collection through collaborations with researchers at outside institutions. Another theoretical concern is that the test finds target sequences in patients without infections or normal controls. RT-qPCR holds a distinct advantage over endpoint PCR, so the inventors can establish a threshold cutoff for test positivity using relative abundance measurements of targets by the ΔCt calculation: Ct of assay−Ct of actin gene.

### Example 8

Deep RNA Sequencing can Identify RNA from Pathogens of Interest

Deep RNA sequencing data was taken from two patients with bacteremia due to Escherichia coli infection. The unmapped reads were aligned to the Escherichia coli genome. Each patient had reads that aligned to fourteen genes in TABLE 9. Bacterial ribosomal RNA was identified because the depletion kits are designed for human ribosomal RNA. Although previous work has looked at ribosomal RNA for pathogen identification, this method is different because the inventors are looking at RNA and not DNA so that the inventors can look for actively expressed genes. Like the probe design for SARS-CoV-2 above, the inventors identify an exact region of the gene of interest covered by the RNA reads identified by the sequencing data. They can also target multiple genes with PCR based on those with the most reads in sick patients. Interestingly, the patient with more reads died, while the other patient survived. This increase in read counts based on the clinical deterioration could be akin to a molecular equivalent of time to a positive culture, which is sometimes used clinically. Bläckberg et al. (2022).

Patient 2 died of an ESBL Escherichia coli bacteremia. In this patient, genes CTX-M (twelve counts) and blaCTX-M (twelve counts) were identified. These genes result in an ESBL pathogen, confirming the culture diagnosis.

These data demonstrate the ability to isolate RNA from the blood, sequence the RNA, and use computational approaches to identify bacterial sequences and create PCR primers to identify infection and resistance.

TABLE 9
Reads of E coli genes per patient with E coli bacteremia

| Gene | Patient 1 | Patient 2 |
|---|---|---|
| rrlA 23S | 244 | 12645 |
| rrsB 16S | 385 | 12274 |
| rrlC 23S | 232 | 12205 |
| rrsE 16S | 384 | 12179 |
| rrsA 16S | 369 | 12036 |
| rrsC 16S | 446 | 11447 |
| rrlE 23S | 282 | 11044 |
| rrlB 23S | 294 | 10629 |
| rrlD 23S | 248 | 10462 |
| rrlH 23S | 208 | 10335 |
| rrsG 16S | 314 | 9875 |
| rrsD 16S | 308 | 9717 |
| rrlG 23S | 285 | 9340 |
| rrsH 16S | 444 | 7912 |
| (bla)CTX-M | Negative | Positive |

### Example 9

### Test Characteristics

TABLE 10
Test Characteristics of Greatest Importance

| Description | Application in this Invention |
|---|---|
| Rapid: proposed ID test time of <4 | A PCR-based test to return a result in less than |
| hours for clinical diagnostics that | four hours. This test is done on a sample |
| differentially detect and identify | directly from blood by looking at the RNA |
| species and concurrently or | present from the bacteria to detect and identify |
| sequentially provide phenotypic | the species. Because it is looking at RNA, not |
| resistance profile information. | DNA, a phenotypic resistance profile is achieved. |
| Culture-independent: the diagnostic | The pathogen identification results from |
| should focus on direct detection of | information obtained directly from the blood. |
| the target pathogens from primary | The PCR targets pathogen RNA, more |
| samples | abundant than single copies of DNA per |
| | organism. Culture is not needed. |
| Technologies capable of detecting | From samples from patients with infections due |
| drug resistance/drug-susceptibility, | to resistant organisms, RNAs corresponding to |
| relevant to clinical decision making | the resistance gene are measured in the PCR. |
| | Resistant genes are selected that alter |
| | antimicrobial therapy for that pathogen if they |
| | are detected. |
| Sensitive and specificity should | Sensitivity and specificity are studied and |
| equal or exceed the sensitivity and | reported according to the Statistical Guidance |
| specificity of FDA-cleared tests for | on Reporting Results from Studies Evaluating |
| proposed agents from the same | Diagnostic Tests - Guidance for Industry and |
| sample type. | FDA Staff. Culture-based tests are performed in |
| | parallel with the molecular tests being developed. |

### Other Embodiments

Specific compositions and methods are disclosed. The scope of the invention should be defined solely by the claims. Persons having ordinary skill in the biomedical art will interpret all claim terms in the broadest possible manner consistent with the context and the spirit of the disclosure. The detailed description in this specification is illustrative and not restrictive or exhaustive. This invention is not limited to the particular methodology, protocols, and reagents described in this specification and can vary in practice. When the specification or claims recite ordered steps or functions, alternative embodiments might perform their functions in a different order or substantially concurrently. Other equivalents and modifications besides those already described are possible without departing from the inventive concepts described in this specification, as persons having ordinary skill in the biomedical art recognize.

When the specification provides a range of values, each intervening value between the upper and lower limit of that range is within the range of values unless the context dictates otherwise.

### CITATION LIST

Persons having ordinary skill in the biomedical art can use these patents, patent applications, and scientific references as guidance to predictable results when making and using the invention.

### Patent Literature

International Pat. Publ. WO 2021/163692 A1 (Rhode Island Hospital), RNA sequencing to diagnose sepsis, published Aug. 19, 2021. Deep RNA sequencing is a technology that provides an initial diagnostic for sepsis that can also monitor the indicia of treatment and recovery (bacterial counts reduce, physiology returns to steady-state).
U.S. Pat. Publ. US 2013/0316331 A1 (Isakof et al.), Detection of infection by a microorganism using small RNA sequencing subtraction and assembly, published Nov. 11, 2013. A method for the detection and identification of infection of a subject by a microorganism, wherein the method is based on the use of small RNA derived sequences and subtraction and assembly thereof.
U.S. Pat. Publ. US 2016/0292356 A1 (Kim et al.), Methods and processes for non-invasive assessment of chromosome alterations, published Oct. 6, 2016. Methods, processes, systems, machines, and apparatuses for non-invasive assessment of chromosome alterations.

### Non-Patent Literature

Acevedo et al., Infectivity and immune escape of the new SARS-CoV-2 variant of interest Lambda. medRxiv, 2021.06.28.21259673 (2021).
Al-Hasan, Winders, Bookstaver, & Justo, Direct measurement of performance: A new era in antimicrobial stewardship. Antibiotics (Basel, Switzerland). 8(3) (2019).
Andrei & Wang, Cutaneous light chain amyloidosis with multiple myeloma: A concise review. Hematology/Oncology and Stem Cell Therapy 12, 71-81 (2019).
Bajaj, Singh, & Virdi, Escherichia coli β-lactamases: What really matters. Frontiers in Microbiology, 7, 417 (2016).
Barnes et al., SARS-CoV-2 neutralizing antibody structures inform therapeutic strategies. Nature, 588, 682-7 (2020).
Based on Science, What is the connection between COVID-19 and sepsis? National Academies (Feb. 14, 2022).
Bläckberg et al., Time to blood culture positivity: An independent predictor of mortality in Streptococcus pyogenes bacteremia. Open Forum Infectious Diseases, 9(6), ofac163 (2022).
Blenkiron et al., Uropathogenic Escherichia coli releases extracellular vesicles that are associated with RNA. PLOS One, 11(8), e0160440 (2016).
Bone et al., Definitions for sepsis and organ failure and guidelines for the use of innovative therapies in sepsis. Chest, 101, 1644-1655 (1992).
Bonine et al., Impact of delayed appropriate antibiotic therapy on patient outcomes by antibiotic resistance status from serious gram-negative bacterial infections. The American Journal of the Medical Sciences, 357(2), 103-10 (2019).
Bortolaia et al., ResFinder 4.0 for predictions of phenotypes from genotypes. The Journal of Antimicrobial Chemotherapy, 75(12), 3491-500 (2020).
Bustin & Huggett, qPCR primer design revisited. Biomolecular detection and quantification, 14, 19-28 (2017).
Bustin, Mueller, & Nolan, Parameters for successful PCR primer design. Methods in Molecular Biology (Clifton, NJ), 2065, 5-22 (2020).
Camargo et al., Next-generation sequencing of microbial cell-free DNA for rapid noninvasive diagnosis of infectious diseases in immunocompromised hosts. F1000Research, 8, 1194 (2019).
Cass & Xiao X, mountainClimber identifies alternative transcription start and polyadenylation sites in RNA-Seq. Cell Systems, 9(4), 23, 393-400.e6 (October 2019).
Chambers & Deleo, Waves of resistance: Staphylococcus aureus in the antibiotic era. Nature Reviews Microbiology, (9), 629-41 (2009).
Chambers, Methicillin resistance in staphylococci: molecular and biochemical basis and clinical implications. Clinical Microbiology Reviews, 10(4), 781-91 (1997).
Cheng et al., Contribution of coagulases towards Staphylococcus aureus disease and protective immunity. PLOS Pathogens, 6(8), e1001036 (2010).
Clatworthy, Pierson, & Hung, Targeting virulence: A new paradigm for antimicrobial therapy. Nature Chemical Biology; 3(9), 541-8 (2007).
Covert, Bashore, Edds, & Lewis, Utility of the respiratory viral panel as an antimicrobial stewardship tool. Journal of Clinical Pharmacy and Therapeutics, 46(2), 277-85 (2021).
D'Mello et al., An in vivo atlas of host-pathogen transcriptomes during Streptococcus pneumoniae colonization and disease. Proceedings of the National Academy of Sciences, 117:33507-18 (2020).
De Bus et al., Antimicrobial de-escalation in the critically ill patient and assessment of clinical cure: the DIANA study. Intensive Care Medicine, 46(7), 1404-17 (2020).
Dersch, Khan, Mühlen, & Görke, Roles of regulatory RNAs for antibiotic resistance in bacteria and their potential value as novel drug targets. Frontiers in Microbiology, 8, 803 (2017).
Dingle et al., Prevalence and characterization of the cefazolin inoculum effect in North American methicillin-susceptible Staphylococcus aureus isolates. Journal of Clinical Microbiology, e0249521 (2022).
Dobin et al., STAR: Ultrafast universal RNA-seq aligner. Bioinformatics (Oxford, England), 29(1), 15-21 (2013).
Dong & Gardner, An interactive web-based dashboard to track COVID-19 in real time. The Lancet, Infectious Diseases (2020).
Drews et al., Resistance of SARS-CoV-2 beta and gamma variants to plasma collected from Canadian blood donors during the Spring of 2020. Transfusion (2021).
Duncan, Youngstein, Kirrane, & Lonsdale, Diagnostic challenges in sepsis. Current Infectious Disease Reports, 23(12), 22 (2021).
Easterday, Van Ert, Zanecki, & Keim, Specific detection of Bacillus anthracis using a TaqMan mismatch amplification mutation assay. BioTechniques, 38(5), 731-5 (2005).
Eizenga et al., Pangenome graphs. Annual Review of Genomics and Human Genetics, 21, 139-62 (2020).
Evans et al., Surviving Sepsis Campaign: International guidelines for management of sepsis and septic shock 2021. Critical Care Medicine, 49(11), e1063-e143 (2021).
FastQC. A quality control tool for high throughput sequence data. (2014).
Ferguson et al., The Berlin definition of ARDS: An expanded rationale, justification, and supplementary material. Intensive Care Medicine, 8(10), 1573-82 (2012).
Fredericks et al., Deep RNA sequencing of intensive care unit patients with COVID-19, Scientific Reports, 12(1) (January 2022).
Glaab & Skopek, A novel assay for allelic discrimination that combines the fluorogenic 5′ nuclease polymerase chain reaction (TaqMan) and mismatch amplification mutation assay. Mutation Research, 430(1), 1-12 (1999).
Global burden of bacterial antimicrobial resistance in 2019: A systematic analysis. Lancet (London, England), 399(10325), 629-55 (2022).
Guo et al., Prevalence and therapies of antibiotic-resistance in Staphylococcus aureus. Frontiers in Cellular and Infection Microbiology, 10, 107 (2020).
Gutmann, Williamson, Collatz, & Acar, Mechanisms of beta-lactam resistance in Haemophilus influenzae. European Journal of Clinical Microbiology & Infectious Diseases: Official publication of the European Society of Clinical Microbiology, 7(5), 610-5 (1988).
Harbottle, Thakur, Zhao, & White, Genetics of antimicrobial resistance. Animal Biotechnology, 17(2), 111-24 (2006).
Hardwick et al., “Getting the entire message: Progress in isoform sequencing.” Frontiers in Genetics, 10, 709 (2019).
Hui, DelMonte, & Ranade, Genotyping using the TaqMan assay. Current Protocols in Human Genetics, Chapter 2: Unit 2.10 (2008).
Islam et al., Chromosomal mechanisms of aminoglycoside resistance in Pseudomonas aeruginosa isolates from cystic fibrosis patients. Clinical Microbiology and Infection: Official publication of the European Society of Clinical Microbiology and Infectious Diseases, 15(1), 60-6 (2009).
Jacoby, AmpC beta-lactamases. Clinical Microbiology Reviews. 22(1), 161-82 (2009).
Karczmarczyk et al., Mechanisms of fluoroquinolone resistance in Escherichia coli isolates from food-producing animals. Applied and Environmental Microbiology, 77(20), 7113-20 (2011).
Kassakian & Mermel, Changing epidemiology of infections due to extended spectrum beta-lactamase producing bacteria. Antimicrobial Resistance and Infection Control, 3(1), 9 (2014).
Krause et al., SARS-CoV-2 variants and vaccines. New England Journal of Medicine, 385, 179-86 (2021).
Kukurba & Montgomery, “RNA sequencing and analysis.” Cold Spring Harbor Protocols, 2015(11), 951-69 (Apr. 13, 2015).
Kumar et al., Duration of hypotension before initiation of effective antimicrobial therapy is the critical determinant of survival in human septic shock. Critical Care Medicine, 34(6), 1589-96 (2006).
Langelier et al., Detection of pneumonia pathogens from plasma cell-free DNA. American Journal Of Respiratory and Critical Care Medicine, 201(4), 491-5 (2020).
Livak, Allelic discrimination using fluorogenic probes and the 5′ nuclease assay. Genetic Analysis: Biomolecular Engineering, 14(5-6), 143-9 (1999).
Lobo & Warwicker, Predicted pH-dependent stability of SARS-CoV-2 spike protein trimer from interfacial acidic groups. Computational and Structural Biotechnology Journal, 19, 5140-8 (2021).
Lu et al., Discovering common pathogenetic processes between COVID-19 and sepsis by bioinformatics and system biology approach. Frontiers in Immunology, Volume 13, Article 975848. (Aug. 31, 2022).
Mandric et al. Profiling immunoglobulin repertoires across multiple human tissues using RNA sequencing. Nature Commun., 11, 3126 (2020).
Mangul et al., ROP: Dumpster diving in RNA-sequencing to find the source of 1 trillion reads across diverse adult human tissues. Genome Biology, 19, 36 (2018).
Marini et al., AMR-meta: A k-mer and metafeature approach to classify antimicrobial resistance from high-throughput short-read metagenomics data. GigaScience, 11 (2022).
Mei-Sheng, Riley & Olans, Implementing an antimicrobial stewardship program in the intensive care unit by engaging critical care nurses. Critical Care Nursing Clinics of North America, 33(4), 369-80 (2021).
Modi & Kovacs, Hospital-acquired and ventilator-associated pneumonia: Diagnosis, management, and prevention. Cleveland Clinic Journal of Medicine, 87(10), 633-9 (2020).
Monaghan et al., Deep RNA sequencing of intensive care unit patients with COVID-19. medRxiv (2021).
Monaghan et al., Deep RNA sequencing of intensive care unit patients with COVID-19. medRxiv (2021).
Müller & Köhler, B cell superantigens in HIV-1 infection. International Reviews of Immunology, 14, 339-49 (1997).
Noureen, Tada, Kawashima, & Arita, Rearrangement analysis of multiple bacterial genomes. BMC Bioinformatics, 20(Suppl 23), 631 (2019).
Olwal et al., Parallels in sepsis and COVID-19 conditions: Implications for managing severe COVID-19. Frontiers in Immunology, Volume 12, Article 602848 (February 2021).
Opota, Jaton, & Greub, Microbial diagnosis of bloodstream infection: towards molecular diagnosis directly from blood. Clinical Microbiology and Infection: the official publication of the European Society of Clinical Microbiology and Infectious Diseases, 21(4), 323-31 (2015).
Palavecino, Rapid methods for detection of MRSA in clinical specimens. Methods in Molecular Biology (Clifton, NJ), 2069, 29-45 (2020).
Paterson et al., Outcome of cephalosporin treatment for serious infections due to apparently susceptible organisms producing extended-spectrum beta-lactamases: implications for the clinical microbiology laboratory. Journal of Clinical Microbiology, 39(6), 2206-12 (2001).
Rappazzo et al., Broad and potent activity against SARS-like viruses by an engineered human monoclonal antibody. Science, 371, 823-9 (2021).
Reynolds et al., Renin-angiotensin-aldosterone system inhibitors and risk of Covid-19. The New England Journal of Medicine, 382, 2441-8 (2020).
Rhee et al., Prevalence of antibiotic-resistant pathogens in culture-proven sepsis and outcomes associated with inadequate and broad-spectrum empiric antibiotic use. JAMA Network Open., 3(4), e202899 (2020).
Rivera-Izquierdo et al., OXA-48 carbapenemase-producing enterobacterales in Spanish hospitals: An updated comprehensive review on a rising antimicrobial resistance. Antibiotics (Basel, Switzerland), 10(1) (2021)
Rudd et al., Global, regional, and national sepsis incidence and mortality, 1990-2017: Analysis for the Global Burden of Disease Study. Lancet (London, England), 395(10219), 200-11 (2020).
Rupp & Fey, Extended spectrum beta-lactamase (ESBL)-producing Enterobacteriaceae: Considerations for diagnosis, prevention and drug treatment. Drugs, 63(4), 353-65 (2003).
Salah et al., Distribution of quinolone resistance gene (qnr) in ESBL-producing Escherichia coli and Klebsiella spp. in Lomé, Togo. Antimicrobial Resistance and Infection Control, 8, 104 (2019).
Schmidt et al., High genetic barrier to SARS-CoV-2 polyclonal neutralizing antibody escape. Nature (2021).
Seok, Jeon, & Park, Antimicrobial therapy and antimicrobial stewardship in sepsis. Infection & Chemotherapy, 52(1), 19-30 (2020).
Sethuraman & Ryo, Interpreting Diagnostic Tests for SARS-CoV-2. JAMA (2020).
Shankar-Hari et al., Developing a new definition and assessing new clinical criteria for septic shock: For the Third International Consensus Definitions for Sepsis and Septic Shock (Sepsis-3). JAMA, 315(8), 775-87 (2016).
Shrestha, Tedla, & Bull, Broadly neutralizing antibodies against emerging SARS-CoV-2 variants. Frontiers in Immunology, 12, 752003 (2021).
Sidstedt et al., Inhibition mechanisms of hemoglobin, immunoglobulin G, and whole blood in digital and real-time PCR. Analytical and Bioanalytical Chemistry, 410(10), 2569-83 (2018).
Singer et al., The third international consensus definitions for sepsis and septic shock (Sepsis-3). JAMA, 315, 801-810 (February 2016).
Stark et al., “RNA sequencing: the teenage years.” Nature Reviews Genetics (2019).
Starr et al., Prospective mapping of viral mutations that escape antibodies used to treat COVID-19. Science, 371, 850-4 (2021).
Starr et al., SARS-CoV-2 RBD antibodies that maximize breadth and resistance to escape. Nature (2021).
Sterne-Weiler et al., Efficient and accurate quantitative profiling of alternative splicing patterns of any complexity on a laptop. Molecular Cell, 72(1), 187-200.e6 (2018).
Strich, Heil, & Masur, Considerations for empiric antimicrobial therapy in sepsis and septic shock in an era of antimicrobial resistance. The Journal of Infectious Diseases, 222(Suppl 2), S119-s31 (2020).
Suzuki, Horinouchi, & Furusawa, Prediction of antibiotic resistance by gene expression profiles. Nature Communications, 5, 5792 (2014).
Tada, Tanizawa, & Arita, Visualization of consensus genome structure without using a reference genome. BMC genomics, 18(Suppl 2), 208 (2017).
Taniguchi et al., Quantifying E. coli proteome and transcriptome with single-molecule sensitivity in single cells. Science (New York, NY), 329(5991), 533-8 (2010).
Tchesnokova et al., Pandemic fluoroquinolone resistant Escherichia coli clone ST1193 emerged via simultaneous homologous recombinations in 11 gene loci. Proceedings of the National Academy of Sciences of the United States of America, 116(29), 14740-8 (2019).
Teixeira et al., Aminoglycoside resistance genes in Pseudomonas aeruginosa Isolates from Cumana, Venezuela. Revista do Instituto de Medicina Tropical de Sao Paulo, 58, 13 (2016).
Terrier et al., Serum biomarker signature identifies patients with B-cell non-Hodgkin lymphoma associated with cryoglobulinemia vasculitis in chronic HCV infection. Autoimmunity Reviews, 13, 319-26 (2014).
Teshome et al., Duration of exposure to antipseudomonal β-lactam antibiotics in the critically ill and development of new resistance. Pharmacotherapy, 39(3), 261-70 (2019).
Tindal et al., Emerging therapeutic targets for sepsis, Expert Opinion on Therapeutic Targets, 25(3), 175-189 (Mar. 4, 2021.)
Tristram, Jacobs, & Appelbaum, Antimicrobial resistance in Haemophilus influenzae. Clinical Microbiology Reviews, 20(2), 368-89 (2007).
Vaduganathan et al., Renin-angiotensin-aldosterone system inhibitors in patients with Covid-19. The New England Journal of Medicine, 382, 1653-9 (2020).
Vazquez-Guillamet et al., Using the number needed to treat to assess appropriate antimicrobial therapy as a determinant of outcome in severe sepsis and septic shock. Critical Care Medicine, 42(11), 2342-9 (2014).
Wang et al., Pseudomonas aeruginosa detection using conventional PCR and quantitative real-time PCR Based on species-specific novel gene targets identified by pangenome analysis. Frontiers in Microbiology, 13, 820431 (2022).
Webb et al., Broad-spectrum antibiotic use and poor outcomes in community-onset pneumonia: a cohort study. The European Respiratory Journal, 54(1) (2019).
Weinreich et al., REGN-COV2, a neutralizing antibody cocktail, in outpatients with Covid-19. New England Journal of Medicine, 384, 238-51 (2020).
Whole-genome sequencing as part of national and international surveillance programmes for antimicrobial resistance: A roadmap. BMJ Global Health, 5(11) (2020).
Willyard, How antiviral pill molnupiravir shot ahead in the COVID drug hunt. Nature (2021).
Wood, Lu, & Langmead, Improved metagenomic analysis with Kraken2. Genome Biology, 20(1), 257 (2019).
Yahav et al., Immunosuppression reduction when administering a booster dose of the BNT162b2 mRNA SARS-CoV-2 vaccine in kidney transplant recipients without adequate humoral response following two vaccine doses: Protocol for a randomised controlled trial (BECAME study). BMJ Open, 11, e055611 (2021).
Yan, Chang, & Wang, Laboratory testing of SARS-CoV, MERS-CoV, and SARS-CoV-2 (2019-nCOV): Current status, challenges, and countermeasures. Reviews in Medical Virology, 30(3), e2106 (2020).
Yang et al., Prevalence and fluoroquinolone resistance of Pseudomonas aeruginosa in a hospital of South China. International Journal of Clinical and Experimental Medicine, 8(1), 1386-90 (2015).
Yu et al., Low prevalence of bloodstream infection and high blood culture contamination rates in patients with COVID-19. PLOS One, 15(11), e0242533 (2020).
Zhang, Yang, & Makam, Utility of blood cultures in pneumonia. The American Journal of Medicine, 132(10), 1233-8 (2019).

### Textbooks and Technical References

Current Protocols in Immunology (CPI) (2003). John E. Coligan, ADA M Kruisbeek, David H Margulies, Ethan M Shevach, Warren Strobe, (eds.) John Wiley and Sons, Inc. (ISBN 0471142735, 9780471142737).
Current Protocols in Molecular Biology (CPMB), (2014). Frederick M. Ausubel (ed.), John Wiley and Sons (ISBN 047150338X, 9780471503385).
Current Protocols in Protein Science (CPPS), (2005). John E. Coligan (ed.), John Wiley and Sons, Inc.
Immunology (2006). Werner Luttmann, published by Elsevier.
Janeway's Immunobiology, (2014). Kenneth Murphy, Allan Mowat, Casey Weaver (eds.), Taylor & Francis Limited, (ISBN 0815345305, 9780815345305).
Laboratory Methods in Enzymology: DNA, (2013). Jon Lorsch (ed.) Elsevier (ISBN 0124199542).
Lewin's Genes XI, (2014). published by Jones & Bartlett Publishers (ISBN-1449659055).
Molecular Biology and Biotechnology: a Comprehensive Desk Reference, (1995). Robert A. Meyers (ed.), published by VCH Publishers, Inc. (ISBN 1-56081-569-8).
Molecular Cloning: A Laboratory Manual, 4th ed., Michael Richard Green and Joseph Sambrook, (2012). Cold Spring Harbor Laboratory Press, Cold Spring Harbor, N.Y., USA (ISBN 1936113414).
Pharmaceutical Sciences 23^{rd} edition (Elsevier, 2020).
The Encyclopedia of Molecular Cell Biology and Molecular Medicine, Robert S. Porter et al., (eds.), published by Blackwell Science Ltd., 1999-2012 (ISBN 9783527600908).
The Merck Manual of Diagnosis and Therapy, 19^{th} edition (Merck Sharp & Dohme Corp., 2018).

All patents and publications cited throughout this specification are expressly incorporated by reference to disclose and describe the materials and methods that might be used with the technologies described in this specification. The publications discussed are provided solely for their disclosure before the filing date. They should not be construed as an admission that the inventors may not antedate such disclosure under prior invention or for any other reason. If there is an apparent discrepancy between a previous patent or publication and the description provided in this specification, the present specification (including any definitions) and claims shall control. All statements as to the date or representation as to the contents of these documents are based on the information available to the applicants and constitute no admission as to the correctness of the dates or contents of these documents. The dates of publication provided in this specification may differ from the actual publication dates. If there is an apparent discrepancy between a publication date provided in this specification and the actual publication date supplied by the publisher, the actual publication date shall control.

TABLE A1
Antibodies to COVID-19

| Antibody | PDB | CDRL3 | SEQ ID NO |
|---|---|---|---|
| Class 1 | | | |
| C102 | 7K8N | CQQYGSSPR | 54 |
| C105 | 6XCM | CSSYEGSNNFV | 55 |
| B38 | 7BZ5 | CQQLNSYPPY | 56 |
| CC12.3 | 6XC7 | CQQYYSTPY | 57 |

Class 2

| | | | |
|---|---|---|---|
| C002 | 7K8O | CQQSYSTPR | 58 |
| C104 | 7K8U | CQQYGTTPR | 59 |
| C119 | 7K8W | CSSYTSSSTSV | 60 |
| C121 | 7K8X | CCSYAGSSTL | 61 |
| C144 | 7K90 | CSSYTSSSTR | 62 |
| COVA2-39 | 7JMP | CCSYAGSSTW | 63 |
| P2B-2F6 | 7BWJ | CSSYAGSNNL | 64 |
| Ab2-4 | 6XEY | CSSYAGSNNL | 65 |

Class 3

| | | | |
|---|---|---|---|
| BD23 | 7BYR | CQQYNSYPY | 66 |
| C135 | 7K8R | CQQYNSYPW | 67 |
| S309 | 6WPS | CQQHDTSL | 68 |
| C110 | 7K8P | CQQYNSYPY | 69 |
| REGN10987 | 6XDG | CQQYDNLPL | 70 |

Class 4

| | | | |
|---|---|---|---|
| CR3022 | 6W41 | CQQYYSTPY | 71 |
| EY6A | 6ZDH | CQQSYSTLAL | 72 |
| S304 | 7JW0 | CQQSYVSPTY | 73 |
| S2A4 | 7JVC | CQSYDSSNHVV | 74 |

Lengthy table referenced here
US20250034234A1-20250130-T00001
Please refer to the end of the specification for access instructions.

Lengthy table referenced here
US20250034234A1-20250130-T00002
Please refer to the end of the specification for access instructions.

TABLE A4
CDR3 Alignements Exclusive to Survivors Day 0

| | Cluster | CDR3 | | | |
|---|---|---|---|---|---|
| Alignment | Counts | Counts | Class | ID | SEQ ID NO |
| CQQYNNYWAF | 43 | 75616 | C3 | C135 | 2 |
| CQQYNNWPPLTF | 52 | 60021 | C3 | REGN10987 | 3 |
| CQQYDNHF | 42 | 48510 | C3 | REGN10987 | 4 |
| CQQYNRYWTF | 26 | 44085 | C3 | C135 | 5 |
| CQQYNNWPPLFTF | 31 | 36103 | C3 | REGN10987 | 6 |
| CQQYNNWPPGFTF | 35 | 35532 | C3 | REGN10987 | 7 |
| CQQLNSYPGGTF | 26 | 35395 | C1 | B38 | 8 |
| CQQYDSYPWTF | 26 | 34909 | C3 | C135 | 9 |
| CQQYNSYSRTF | 32 | 34521 | C3 | C135 | 10 |
| CQQYGSFF | 25 | 33616 | C1 | C102 | 11 |
| CQQYDKWPFF | 25 | 30823 | C4 | S2A4 | 12 |
| CMQALQTPLTF | 18 | 26607 | C2 | C002 | 13 |
| CMQALQTPRTF | 17 | 26599 | C2 | C104 | 14 |
| CQQRSNWPPLTF | 39 | 20622 | C3 | REGN10987 | 15 |
| CHQYNNW | 30 | 19583 | C3 | REGN10987 | 16 |
| CQQFNTWPPE | 17 | 19345 | C1 | B38 | 17 |
| CQQYQTWPPLF | 28 | 17810 | C1 | B38 | 18 |
| CQQFNNWPPGFSF | 17 | 16145 | C1 | B38 | 19 |
| CQSVDSSGIYI | 27 | 15458 | C4 | S2A4 | 32 |
| CQSADSSGTYPDVVF | 33 | 15132 | C4 | S2A4 | 33 |
| CQQYNTYSQHTF | 31 | 13179 | C2 | C104 | 20 |
| CQSYDSSPLF | 49 | 12495 | C4 | S2A4 | 21 |
| CQAWDSST--HVVF | 20 | 10805 | C4 | S2A4 | 24238 |
| CQSTDSSGTFW | 36 | 9902 | C4 | S2A4 | 69507 |
| CQQYNTNSPYNF | 15 | 9606 | C3 | C135 | 4864 |
| CQSYDSSLSGVVF | 31 | 7143 | C4 | S2A4 | 1241 |
| CNSRDSSGNLPF | 27 | 6435 | C4 | S2A4 | 2568 |
| CNSRDSSGNHLVF | 18 | 6320 | C4 | S2A4 | 10139 |
| CQSTDSSGSWLF | 20 | 5327 | C4 | S2A4 | 2855 |
| CQVWDSSSDHVVF | 23 | 4745 | C4 | S2A4 | 2300 |
| CCSYAGSYTF | 16 | 4197 | C2 | COVA2-39 | 6244 |
| CMQGTHWPPYTF | 15 | 4104 | C1 | B38 | 4772 |
| CRQYNNWPRGAF | 21 | 3674 | C3 | REGN10987 | 2745 |
| CQTSDSSGFYPELF | 17 | 3514 | C4 | S2A4 | 5347 |
| IAYYDSSGHYRDYW | 113 | 2298 | C4 | S2A4 | 69508 |
| CQYYDNVP | 14 | 2108 | C3 | REGN10987 | 69509 |
| CHQRGNWPF | 20 | 1839 | C3 | REGN10987 | 2970 |
| CQQRGHWPPNSF | 20 | 1619 | C1 | B38 | 4186 |
| CQQRTTWPPNPF | 20 | 1506 | C1 | B38 | 2518 |
| CQQRSKWPPECSF | 14 | 1368 | C1 | B38 | 18448 |
| CQQRSPWPPMCRF | 10 | 1311 | C1 | B38 | 9961 |
| CQTWGTGIHVVF | 9 | 878 | C2 | C104 | 26483 |
| CQQRANWP | 18 | 861 | C3 | REGN10987 | 69510 |
| CSSYAGSYNVAF | 14 | 785 | C2 | COVA2-39 | 965 |
| CQVWDSSSDHPYYVF | 16 | 636 | C4 | S2A4 | 27034 |
| CQVWDSDSDHAIF | 17 | 629 | C4 | S2A4 | 4828 |
| CQVWDSKSAHGGVF | 13 | 620 | C4 | S2A4 | 11277 |
| CQVWDSRSDHSAWVF | 12 | 597 | C4 | S2A4 | 27802 |
| CQQANSFPRITF | 17 | 536 | C3 | C135 | 4932 |
| ITEWFDPW | 1549 | 336 | None | | 69511 |
| AREYYYYYMDVW | 7648 | 240 | None | | 69512 |
| ARGSYYFDYW | 2343 | 223 | None | | 69513 |
| FGGNPDAFDIW | 1396 | 215 | None | | 69514 |
| ATPYYFDYW | 1296 | 195 | None | | 69515 |
| TINDALDIW | 1138 | 178 | None | | 69516 |
| CTNFDYYYYMDVW | 1575 | 176 | None | | 24550 |
| YGSGSPDAFDIW | 885 | 170 | None | | 69517 |
| CATLGAYYYYYMDVW | 855 | 169 | None | | 26309 |
| CYSSGYYYYYYMDVW | 1611 | 168 | None | | 843 |
| TYYYDSSGYYRGGFDYW | 6743 | 163 | None | | 69518 |
| CAAYHYYPYGMDVW | 724 | 159 | None | | 26995 |
| CAVFTYYYDSSGYYFDYW | 3414 | 153 | None | | 25469 |
| SYDSSGYYLDFDYW | 2389 | 152 | None | | 69519 |
| ASLNDDAFDIW | 620 | 147 | None | | 69520 |
| VPKDSSGYFLFDYW | 2413 | 142 | None | | 69521 |
| SNNYYYYMDVW | 1405 | 141 | None | | 69522 |
| CAS-GYYYDSSGYGIFDYW | 2273 | 135 | None | | 3556 |
| CVKDGGYYYYYMDVW | 7125 | 134 | None | | 9712 |
| CARDS--YYYDSSGYFDYW | 3028 | 132 | None | | 4138 |
| ARKPYYYYYMDVW | 878 | 122 | None | | 69523 |
| ESHKSDAFDIW | 634 | 121 | None | | 69524 |
| YDSSGYHDYW | 2094 | 117 | None | | 69525 |
| CAHIDSSGYYEFDYW | 750 | 116 | None | | 27703 |
| CGRDYYDSSGYYTQY | 2313 | 114 | None | | 69526 |
| GILTGYYTYFDYW | 833 | 112 | None | | 69527 |
| CARENYYDSSGYFRS | 988 | 112 | None | | 69528 |
| CARGPSYYDSSGYPADYW | 2025 | 110 | None | | 2294 |
| SSGYYYGDYW | 696 | 107 | None | | 69529 |
| APYDYDSSGYIPDYW | 2100 | 104 | None | | 69530 |
| CTRDYYDSSGYYYGFDYW | 2443 | 102 | None | | 2368 |
| CASSYYYDSSGQPDYW | 511 | 101 | None | | 3503 |
| CARDDHYYDSSGYDYW | 2191 | 101 | None | | 5240 |
| IYYYDSSGYYWNYW | 2378 | 99 | None | | 69531 |
| CASSYYDSSGYLPVDYW | 2091 | 98 | None | | 24443 |
| TSGLPDPFDIW | 607 | 98 | None | | 69532 |
| CAT-INYYDSSGYYFPFDYW | 2170 | 96 | None | | 26941 |
| CAKIGDFYYYYMDVW | 352 | 96 | None | | 7368 |
| CARDMDYYDSSGYYQDYW | 667 | 96 | None | | 2635 |
| CARDYYDSSGYSRPFDYW | 624 | 96 | None | | 11177 |
| SSSWYGYYFDYW | 1239 | 96 | None | | 69533 |
| AKATPRYYYYGMDVW | 463 | 96 | None | | 69534 |
| CATFP-HYYDSSGYYPFDYW | 2167 | 96 | None | | 1316 |
| CTTRLDYYDSSGYYKPFDYW | 2062 | 95 | None | | 720 |
| CASSGLYYYDSSGYSDYW | 651 | 94 | None | | 369 |
| CARMRAYYYDSSGYNDYW | 2072 | 94 | None | | 394 |
| CASLGYYYDSSGYNQFDYW | 2243 | 94 | None | | 11105 |
| CATIPL-YYYDSSGYSRRFDYW | 583 | 93 | None | | 25518 |
| CARDYYYDSSGLLDYW | 699 | 93 | None | | 24107 |
| ASTTGAYYYYGMDVW | 508 | 93 | None | | 69535 |
| CARGKDYDFWSGYYPIY | 542 | 93 | None | | 69536 |
| CATFPVHYYDSSGYYPFDYW | 559 | 93 | None | | 25397 |
| CARGYYDFWSGLDYW | 394 | 92 | None | | 1648 |
| CAMGDLWFDPW | 366 | 92 | None | | 9602 |
| ARLYDSSGYLDYW | 536 | 90 | None | | 69537 |
| YDSSGYYYLDYW | 3133 | 90 | None | | 69538 |
| GPYYYDSSGYFDYW | 454 | 89 | None | | 69539 |
| CASGSSDNWFDPW | 521 | 89 | None | | 2975 |
| CAHSYYDFWSGYYDYW | 785 | 89 | None | | 5100 |
| CARLIGYYDSSGYYWFDYW | 2139 | 88 | None | | 27148 |
| IAARPYYFDYW | 1212 | 88 | None | | 69540 |
| CANGDYYDSSGAPDYW | 538 | 88 | None | | 11683 |
| CARRGYDSSGY--DAFDIW | 381 | 87 | None | | 11324 |
| CAR---VYSSGWYDDYW | 703 | 87 | None | | 23601 |
| ACYYESSGYRLDYW | 2126 | 86 | None | | 69541 |
| CARDYYDSSGYYNWFDPW | 557 | 85 | None | | 27391 |
| CAR--VGDSDAFDIW | 676 | 85 | None | | 25178 |
| CAGSYSYYDSSGYELPFDYW | 2002 | 85 | None | | 4610 |
| CARQRNYYYDSSGYYLDYW | 675 | 84 | None | | 23901 |
| CSRA--GYYRFDYW | 652 | 84 | None | | 7252 |
| CASSYSYDSSGYY---DYFDYW | 680 | 83 | None | | 761 |
| CAR--GDDFWSGYYDYW | 465 | 83 | None | | 5980 |
| AGPYYYFDIW | 494 | 83 | None | | 69542 |
| CASGFYDSSGYYYGYW | 883 | 82 | None | | 2509 |
| CARGYSIDYW | 2618 | 82 | None | | 24435 |
| CASHIADYYYYGMDVW | 288 | 81 | None | | 2769 |
| ATYYNYDAFDIW | 512 | 81 | None | | 69543 |
| CARVVQDYYDSSGYEDYW | 2089 | 81 | None | | 5193 |
| CARI---VAGYYFDYW | 525 | 81 | None | | 9799 |
| GYSSSSSYFDYW | 2740 | 81 | None | | 69544 |
| ASLSGWYVDYW | 921 | 80 | None | | 69545 |
| YGDYDDAFDIW | 399 | 80 | None | | 69546 |
| CARQAPGYYDSSGYYYVYW | 355 | 80 | None | | 26686 |
| CAHTLTYYYDSSGYYSFDYW | 512 | 80 | None | | 27954 |
| CARDATYYDSSGYYDAFDIW | 2266 | 80 | None | | 23537 |
| CARGS--GNYYFDYW | 1552 | 78 | None | | 27736 |
| CARDDTYYYDSSGYFFDYW | 2040 | 78 | None | | 2429 |
| CARGEFDPW | 1354 | 78 | None | | 2494 |
| CAREGYYYDSSGYSNAFDIW | 519 | 78 | None | | 6711 |
| CAR---FGYYYYYYGMDVW | 537 | 77 | None | | 27728 |
| CARDPGWYYYDSSGLDYW | 1397 | 77 | None | | 244 |
| CAKMGEYYYDSSGYSDYW | 610 | 76 | None | | 3081 |
| CAGYGDYSDYW | 10028 | 76 | None | | 31 |
| CASLTYYYDSSGYSVDYFDYW | 2117 | 76 | None | | 1878 |
| CARDRGTYYYDSSGLDYW | 1395 | 76 | None | | 2410 |
| CAHLVSSGWYGDAFDIW | 372 | 76 | None | | 7328 |
| CAKDGYDFWSGYYNYW | 719 | 75 | None | | 28032 |
| CARDPPYYYDSSGYYGWFDPW | 469 | 74 | None | | 5290 |
| GPNDYW | 514 | 74 | None | | 69547 |
| CARDPYYSSYW | 461 | 74 | None | | 27158 |
| CAGVRGSNNDAFDIW | 383 | 73 | None | | 3784 |
| ASGDSSGSFDYW | 661 | 73 | None | | 69548 |
| CARDSEPYYYDSSGYHDYW | 2069 | 72 | None | | 27586 |
| VVPAAPFYYYYGMDVW | 373 | 72 | None | | 69549 |
| CAREYYYDSSGTFDYW | 1985 | 72 | None | | 2589 |
| CATVYSGSYLDAFDIW | 414 | 72 | None | | 2304 |
| CATGYSSSWYFYW | 1164 | 71 | None | | 25184 |
| WVTTDAFDIW | 412 | 71 | None | | 69550 |
| CAHSSPTYYYAMDVW | 354 | 71 | None | | 24700 |
| CARVNAEYYYDSSGYLDYW | 1967 | 71 | None | | 3364 |
| CAK-RYSSGWYGVDYW | 1253 | 71 | None | | 6606 |
| CAKDRYYYDSSGYTYYFDYW | 571 | 71 | None | | 1429 |
| CATH---DFWSGYTFDYW | 825 | 70 | None | | 940 |
| CAADLRNYYDSSGYADYW | 525 | 70 | None | | 4010 |
| CARDFPYYYDSSGYYFDAFDIW | 488 | 70 | None | | 5353 |
| CARDRDSSGYYLDAFDIW | 351 | 70 | None | | 10426 |
| CARDPYYYDSRGYSGDYW | 592 | 70 | None | | 4314 |
| SGSYLSAFDIW | 401 | 70 | None | | 69551 |
| CTSSSWARFDYW | 1239 | 69 | None | | 24627 |
| ATDIPQDYYYGMDVW | 259 | 69 | None | | 69552 |
| CAKRGGGYYYDSSGYLDYW | 2857 | 69 | None | | 1824 |
| CARDGYDSSGYYNWFDPW | 463 | 69 | None | | 4790 |
| CARDNRYYDSSGYSDAFDIW | 490 | 69 | None | | 4818 |
| NYYGSGSYNDYW | 308 | 69 | None | | 69553 |
| CAKDYYDSSGYSDYAFDIW | 395 | 69 | None | | 27039 |
| AFCSGGSCYNWFDPW | 312 | 69 | None | | 69554 |
| CASGYCSGGSCYPPIDYW | 325 | 68 | None | | 2620 |
| CARD-----NSSPYYYYGMDVW | 365 | 68 | None | | 24985 |
| CAKDLGGYYYYW | 571 | 68 | None | | 1060 |
| CARDLGGYFDYW | 560 | 68 | None | | 7037 |
| CAREYYYDSSGYYDSLFDYW | 456 | 67 | None | | 186 |
| CAKSPG---AYYFDYW | 335 | 67 | None | | 4695 |
| CARLSYYDFWSGTGD | 246 | 67 | None | | 69555 |
| GRCSGGSCYSVDYW | 298 | 67 | None | | 69556 |
| CAKTDSSGYYLLDYW | 337 | 66 | None | | 6377 |
| CAKDSQYYYDSSGYYGADYW | 597 | 66 | None | | 2951 |
| CAR-ESGYYYDSSGYQYYFDYW | 2119 | 66 | None | | 3098 |
| CAHRHGYYDAFDIW | 625 | 66 | None | | 6601 |
| CARD----DSSGYLDAFDIW | 314 | 65 | None | | 3974 |
| CARGFSSSWYWAYW | 581 | 65 | None | | 5189 |
| CARDFCSGGSCYPDYW | 284 | 65 | None | | 5228 |
| CSSTSCQGAFDIW | 255 | 65 | None | | 11210 |
| CARTPGYCSSTSCYTYFDYW | 294 | 65 | None | | 10548 |
| CAHRP--SGWIDAFDIW | 401 | 65 | None | | 326 |
| CAHRGYDSSG--VFDYW | 1952 | 65 | None | | 1354 |
| SSRGYYYGMDVW | 291 | 64 | None | | 69557 |
| CARGVYCSGGSCYSVVDYW | 282 | 64 | None | | 9752 |
| CARVDDSSGYYLDAFDIW | 305 | 64 | None | | 5106 |
| CARVLSYYYDSSGYDHFDYW | 1937 | 64 | None | | 10038 |
| CAKGTYCSGGSCY-IFDYW | 347 | 64 | None | | 6993 |
| IQNFDYW | 527 | 64 | None | | 69558 |
| CARSPYSYDSSGYYFDYW | 1999 | 64 | None | | 1143 |
| CARAKQYYYDSSGYYFGYW | 465 | 64 | None | | 3897 |
| CARRALGYCSGGSCYSA-FDYW | 298 | 63 | None | | 6977 |
| DSVLYYFDYW | 441 | 63 | None | | 69559 |
| SYYDSSGYYLFDYW | 344 | 63 | None | | 69560 |
| QGGYSNAFDIW | 328 | 63 | None | | 69561 |
| CARVNNYDFWSGYYTY | 626 | 63 | None | | 69562 |
| CARDPATYYYDSSGTYFDYW | 1976 | 63 | None | | 412 |
| TALWSYYYYYMDVW | 323 | 63 | None | | 69563 |
| TSRSDSSGPFFDYW | 371 | 63 | None | | 69564 |
| CARVHYYDSSGYPRIDYW | 262 | 62 | None | | 2898 |
| CARGEVDYYDSSGYPHFDYW | 1904 | 62 | None | | 27890 |
| CAKDTYYDSSGVDYW | 394 | 62 | None | | 2856 |
| CAKDSLLYYDSSGYFDYW | 1941 | 62 | None | | 4404 |
| CARYPFLYYDSSGYFDYW | 1814 | 61 | None | | 9745 |
| CARSRTSYYDSSGYFLHW | 611 | 61 | None | | 24265 |
| CARQRLNYYDSSGYYYFDYW | 1989 | 61 | None | | 1445 |
| CARDTPPTYYYDSSGYPDYW | 1341 | 61 | None | | 2682 |
| CARDQRGYYDSSGYFDYW | 1842 | 61 | None | | 6456 |
| AGYYARFDYW | 415 | 61 | None | | 69565 |
| CATLGDYGDYYFDYW | 640 | 61 | None | | 3888 |
| SSYFYYAVDVW | 424 | 60 | None | | 69566 |
| HSSGWNWYFDYW | 379 | 60 | None | | 69567 |
| CARDLRYCSGGSCYSAYFDYW | 295 | 60 | None | | 4082 |
| CARGSAIYYDSSGYFDYW | 1869 | 60 | None | | 25974 |
| CAH-YYDILTGYPLFDYW | 939 | 60 | None | | 1921 |
| CAKDRYSSGWYYFDYW | 1209 | 60 | None | | 6654 |
| GGSSHFDYW | 941 | 59 | None | | 69568 |
| AGIFGRWFDPW | 264 | 59 | None | | 69569 |
| CAR---VYSSGWSDAFDIW | 522 | 59 | None | | 225 |
| CAKE-TYYYDSSGYRLLDYW | 1152 | 59 | None | | 1526 |
| ATYYYDSSGTYWYFDLW | 330 | 59 | None | | 69570 |
| HDSSGYSDYW | 1869 | 58 | None | | 69571 |
| CAKVLYYYDSSGSLDYW | 399 | 58 | None | | 1257 |
| CARFYCSSTSCHFDYW | 332 | 58 | None | | 11478 |
| CATRVTGGVDAFDIW | 305 | 58 | None | | 24921 |
| CATVPRYCSSTSCYSGAFDIW | 301 | 57 | None | | 27426 |
| CARVSQGYYDSSGYHYFDYW | 1899 | 57 | None | | 5033 |
| CARPSRYCSGGSCYPAFDYW | 262 | 57 | None | | 27113 |
| CAKVDFWSGYSPDYW | 305 | 57 | None | | 3882 |
| CAREHPDYYDSSGCNDYW | 266 | 57 | None | | 3402 |
| CARAGGYCSSTSC---FDYW | 282 | 57 | None | | 26790 |
| CARGDPYDFWSGPPDYW | 295 | 57 | None | | 7685 |
| CAR-EYYDSSGYPTIDYW | 264 | 57 | None | | 459 |
| GTFDPW | 1231 | 57 | None | | 69572 |
| CARGLSSSWYDYW | 606 | 57 | None | | 3625 |
| CTRDS--GYNAFDIW | 252 | 57 | None | | 11554 |
| AYDSSGYYGLSDAFDIW | 243 | 56 | None | | 69573 |
| SSGWYPRLDYW | 905 | 56 | None | | 69574 |
| GVYYDSDGYYHAFDIW | 423 | 56 | None | | 69575 |
| CASFVRYCSSTSCYHFDYW | 270 | 56 | None | | 1081 |
| CARDDDYYDSSGYILPFDYW | 385 | 56 | None | | 3872 |
| RLDGDNWFDPW | 229 | 56 | None | | 69576 |
| ATDCSGGSCYSNYW | 249 | 55 | None | | 69577 |
| CASDRSVYYYDSSGYDYW | 351 | 55 | None | | 2164 |
| CAKDPFYDFWSGYYFDYW | 607 | 55 | None | | 1399 |
| CARVDT---YYMDVW | 6598 | 55 | None | | 23569 |
| CAKGLYDFWSGIDYW | 266 | 55 | None | | 3569 |
| CAREGDYYDSSGYYYTGFDYW | 593 | 55 | None | | 957 |
| CARDGIYYYDSSGYYSDAFDIW | 664 | 55 | None | | 27874 |
| CATSP---SSGWLPFDYW | 754 | 55 | None | | 25634 |
| YYYDSSGYQDAFHIW | 325 | 55 | None | | 69578 |
| CAHSGSSGRYFDYW | 798 | 55 | None | | 10706 |
| AREYDFWSGYYTGMDYW | 309 | 54 | None | | 69579 |
| CARQSLGYYDSSGYPPFDYW | 1799 | 54 | None | | 23595 |
| VRQTRSWFDPW | 454 | 54 | None | | 69580 |
| CAKGLYYDSSGYPTFFDYW | 267 | 54 | None | | 27323 |
| CARAS-SKYYDSSGYHPDYW | 1984 | 54 | None | | 26388 |
| ETDYYDSSGYPGFDYW | 307 | 54 | None | | 69581 |
| CARDV-YCSGGSCYSVPFDYW | 249 | 54 | None | | 2533 |
| CARVPLSYYYYHMDVW | 530 | 54 | None | | 24803 |
| ATASAYWYFDLW | 421 | 54 | None | | 69582 |
| CASNYYDSSGLYWGYW | 1973 | 54 | None | | 2237 |
| REQGNYFDYW | 311 | 53 | None | | 69583 |
| CASWSDGEDAFDIW | 215 | 53 | None | | 6876 |
| YDSSGYWGDAFDIW | 190 | 53 | None | | 69584 |
| CAVCYSNSHY-FDYW | 915 | 53 | None | | 1266 |
| CARHV--WEYYYDSSGYYDAFDIW | 308 | 53 | None | | 25502 |
| CARAPNYYDSSGYPLDAFDIW | 237 | 53 | None | | 242 |
| CAGGNGSPNWFDPW | 512 | 53 | None | | 9727 |
| CARH-YYYDSSGYSGAVDYW | 378 | 53 | None | | 1541 |
| CARTFAVYYYDSSGIDYW | 301 | 52 | None | | 4897 |
| CVRETYYYDSSGY--YIDPW | 423 | 52 | None | | 5095 |
| CSGGSCPLFDYW | 277 | 52 | None | | 69585 |
| TRQYYGSFDYW | 293 | 51 | None | | 69586 |
| CARLPRYYDSSGWYFDYW | 1872 | 51 | None | | 3165 |
| CARDLGYCSGGNCYSPDYW | 248 | 51 | None | | 27361 |
| CAKYYDFWSGYP-WFDPW | 264 | 51 | None | | 4387 |
| CARVHILYYDSSGYFAYW | 1876 | 51 | None | | 26433 |
| CARRRNYYDSSGYPLDAFDIW | 219 | 51 | None | | 27049 |
| CATWGYSSGWYNNWFDPW | 477 | 51 | None | | 526 |
| CARDSGSSWYFDLW | 252 | 50 | None | | 24524 |
| TTVRAWYYFDYW | 828 | 50 | None | | 69587 |
| CAVIIAVAGTD-FDYW | 289 | 50 | None | | 1831 |
| CASGANYYDSSGYYRLLDYW | 259 | 50 | None | | 25852 |
| CARDPLSGVYYYYYGMDVW | 485 | 50 | None | | 27491 |
| MVITPYYFDYW | 374 | 50 | None | | 69588 |
| CARHQYYYDRSGYYPDYW | 516 | 50 | None | | 10607 |
| CAKLS-GSYHNFDYW | 347 | 50 | None | | 3847 |
| CATSPGGYSSGWYYFDYW | 319 | 49 | None | | 294 |
| CAKDSAYCSGGSCY-GFDYW | 219 | 49 | None | | 26893 |
| CARGLPRYYDFWSGYSA | 242 | 49 | None | | 69589 |
| CAREDCSGGSCYFDYW | 656 | 49 | None | | 24365 |
| CAKFSDCSGGSCYSRL-DYW | 226 | 49 | None | | 25526 |
| CARAGYCSGGSCNYYFDYW | 794 | 49 | None | | 9599 |
| ARLPSGRYNWFDPW | 200 | 49 | None | | 69590 |
| CASAGSGSYQ-DYW | 524 | 49 | None | | 78 |
| CAHQASSGWYGYYFDYW | 566 | 48 | None | | 7387 |
| CARRYCSGGSCSLDYW | 196 | 48 | None | | 10397 |
| CARGDILTGYYPFDYW | 972 | 48 | None | | 3382 |
| CARGYTSGWYDFDYW | 415 | 48 | None | | 4755 |
| CAKLSSWYTPFDYW | 201 | 48 | None | | 1837 |
| CATGPNYYDSSGYPNWFDPW | 224 | 47 | None | | 3929 |
| CATIAAAGPDYYYYYGMDVW | 267 | 47 | None | | 2097 |
| CARFSWQMDAFDIW | 203 | 47 | None | | 5118 |
| CARDRGTYDSSGYYY-LDYW | 297 | 47 | None | | 24840 |
| CARDYSSGWYLAFDIW | 319 | 47 | None | | 26629 |
| CAKDPTGYYYDSSGYYYDYW | 460 | 47 | None | | 3990 |
| CARLGSGFQDAFDIW | 270 | 46 | None | | 28008 |
| AGSSDYW | 341 | 46 | None | | 69591 |
| CARDPVTGY-YYYYMDVW | 359 | 46 | None | | 10113 |
| KAAGPYNWFDPW | 187 | 46 | None | | 69592 |
| CARRAPYYYDSSGPYAFDIW | 241 | 46 | None | | 5218 |
| CARDYHDFWSGYHT---DYW | 356 | 46 | None | | 26710 |
| CARDPPYDSSGYYGYYFDYW | 417 | 46 | None | | 1228 |
| CARDEWDYYYYYGMDVW | 208 | 46 | None | | 7035 |
| CAKAGYRCSGGSCYSYYFDYW | 211 | 46 | None | | 2272 |
| CVKVSPGYYDSSGY-YYDYW | 372 | 46 | None | | 10948 |
| CARDDYG--GNYYYYGMDVW | 389 | 46 | None | | 24908 |
| ANSGNHFDYW | 327 | 46 | None | | 69593 |
| TTGYYLGAFDIW | 192 | 45 | None | | 69594 |
| CARDFDDYDSSGYYGAFDIW | 279 | 45 | None | | 2090 |
| CARHSYYDSSGYPLFYFDYW | 287 | 45 | None | | 119 |
| AKGQEPYYYYTMDVW | 197 | 45 | None | | 69595 |
| CAATYYDILTGYL---DYW | 711 | 45 | None | | 6467 |
| CAKDKLYYYYDSSGYYDYW | 355 | 45 | None | | 6706 |
| CAHGGSYSNYYFDYW | 199 | 45 | None | | 25690 |
| CAAYGDEGGYFDYW | 394 | 45 | None | | 6782 |
| CARDPAYYYDSSG---SDAFDIW | 254 | 45 | None | | 1466 |
| CARNSEGNWFDPW | 367 | 45 | None | | 24789 |
| GITLHYFDYW | 184 | 44 | None | | 69596 |
| TTDVGYGWFDPW | 175 | 44 | None | | 69597 |
| CARDLGYSSGWPFDYW | 414 | 44 | None | | 24873 |
| CAMSTGYYWTFDYW | 357 | 44 | None | | 6875 |
| TTVYSSSWYWYFDLW | 338 | 44 | None | | 69598 |
| CARDYYDSSGYSDGMDVW | 344 | 44 | None | | 7374 |
| SIGGNPPYYYYYMDVW | 281 | 44 | None | | 69599 |
| CARRSYGQYYFDYW | 1564 | 44 | None | | 2835 |
| CAKDIYGSGSYPDVW | 468 | 44 | None | | 4274 |
| CARVVDYDFWSGYAYYFDYW | 565 | 43 | None | | 11446 |
| CARHE-VYDSSGYYFLDYW | 225 | 43 | None | | 27517 |
| CALPIWISFLMCCWIQFASILLRIFALMFIKDIGLKFS | 106 | 43 | None | | 69600 |
| FCVVSLPGF | | | | | |
| CAKGTGYSSGWFDYW | 384 | 43 | None | | 10137 |
| CSSDLFLMCCWICFASILLRILASMFIKDIGLKFSFLV | 106 | 43 | None | | 69601 |
| VSLPGF | | | | | |
| CAGYGDYIEYYFDYW | 570 | 43 | None | | 25205 |
| CAHSDSSGYSYYYYYGMDVW | 218 | 43 | None | | 27645 |
| CARGPDTSGYYY | 501 | 43 | None | | 69602 |
| SWGPRDAFDVW | 151 | 43 | None | | 69603 |
| CARSYSSGWYVGYYFDYW | 246 | 43 | None | | 5069 |
| AGFRDYYDSSGDAFDIW | 175 | 43 | None | | 69604 |
| ISFLKCCWIRFASILLRIFTSMFIRDIGLKFSFFVVSL | 106 | 43 | None | | 69605 |
| PGF | | | | | |
| CATVPREYYYDSSGYYKPFDYW | 283 | 43 | None | | 26367 |
| SGSYSPLDYW | 182 | 43 | None | | 69606 |
| CALPIWISFLICCWIWFASILVRIFASMFIKDIGLKFS | 106 | 43 | None | | 69607 |
| FFVLCLPGF | | | | | |
| CAKDEDCSGGSCYSGAFDIW | 227 | 43 | None | | 1025 |
| CALPIWWISFLMCCWIQFASILLRIFASMFIKDIGLKF | 106 | 43 | None | | 69608 |
| SFFVVSLPGF | | | | | |
| CARGAYCSGGTCYRPFDYW | 189 | 43 | None | | 11244 |
| CAKLGSYYDFWSGYYSQFDYW | 636 | 42 | None | | 1514 |
| CATGWTYYYDSSGYFQHW | 1353 | 42 | None | | 26459 |
| CAR--IPYYGSGSYEDYW | 293 | 42 | None | | 9768 |
| CALPICFLMCCWIQLASILLRIFTSMFIRDIGLKFSFF | 103 | 42 | None | | 69609 |
| VVSLPGF | | | | | |
| CARVRFEDYYYYGMDVW | 236 | 42 | None | | 2967 |
| CARGGDYDFWSGSFDYW | 309 | 42 | None | | 11117 |
| CSSDLLLTCCWIWFASILLRIFASMFIRDIGLKFSFSV | 104 | 42 | None | | 69610 |
| VFLAGF | | | | | |
| CARDSTYYDSSGYIKAFDIW | 319 | 42 | None | | 10813 |
| CALPIWISFLMCCWIWFASILWRIFASMFITDVGLKFS | 102 | 41 | None | | 69611 |
| FFVVSLPGF | | | | | |
| CAHRIY-DSGGYYDYYFDYW | 411 | 41 | None | | 3545 |
| CARIGD-DFWSGYPYYFDYW | 640 | 41 | None | | 265 |
| FLMCCWIQFASILLMIFTSMFIRDIGLKFSFFVVSLSG | 102 | 41 | None | | 69612 |
| F | | | | | |
| CALPILDKLLTCCWIWFASILLRIFASMFIRDIGLKFS | 102 | 41 | None | | 69613 |
| FSVVFLAGF | | | | | |
| CALPIFLMCCWIQFASILLRIFASMFIRDIRLKFSFF | 101 | 41 | None | | 69614 |
| VVSLPGF | | | | | |
| CAR-VVPYYDFWSGSPFDYW | 256 | 41 | None | | 25454 |
| CASLYSSGGRLDYW | 4350 | 41 | None | | 26919 |
| CSSDLLLTCCWIWFASILLRIFASMFIGDIGLKFSFSV | 102 | 41 | None | | 69615 |
| VFLAGF | | | | | |
| CALPIWISFLMCCWIWFASVLLKIFASMFIRDIGLKFSF | 102 | 41 | None | | 69616 |
| FVVSLPGF | | | | | |
| CALPI-LSFLMCCWIWFASISFRIFALMFIRDIGLKFS | 99 | 40 | None | | 69617 |
| FFILSLPGF | | | | | |
| CALPICFLMCCWIWFASILWKIFASMFITDVGLKFSF | 100 | 40 | None | | 69618 |
| FVVSLPGF | | | | | |
| NPPGDYW | 338 | 40 | None | | 69619 |
| HCSSTSCYDAFDIW | 191 | 40 | None | | 69620 |
| CASEIPGEYYFDYW | 754 | 40 | None | | 24345 |
| CALPIWISFLMCCWIWFASISFRIFALMFIRDIGLKFS | 99 | 40 | None | | 69621 |
| FFILSLPGF | | | | | |
| CALPIWISFLMYCWICFASILLRIFALMFIRDIGLKFS | 99 | 40 | None | | 69622 |
| FFVVSLPGF | | | | | |
| CALPIWISFLMCCWTQFASILLRIFASMFIRDIGLKFS | 99 | 40 | None | | 69623 |
| VFVVSLPGF | | | | | |
| CALPI--CFLMYCWICFASILLRIFALMFIRDIGLKFS | 99 | 40 | None | | 69624 |
| FFVVSLPGF | | | | | |
| CAR--YYYDILTGYLHFDYW | 337 | 40 | None | | 5017 |
| CALPIFLMCLSIQFASILLRIFTSMFIRDIGLKFSFFV | 99 | 40 | None | | 69625 |
| VSLPGF | | | | | |
| TVGAGYNAFDIW | 122 | 40 | None | | 69626 |
| CARGTSSGWYFGYW | 420 | 40 | None | | 10936 |
| CALPIWISFSMCYWIRFASILLRIFASMFFRDIGLKFS | 99 | 40 | None | | 69627 |
| FFVVSLPGF | | | | | |
| CARD--YDSSGYYRFFAFDIW | 254 | 39 | None | | 2086 |
| CAKGRCSGGSCYWIDSW | 141 | 39 | None | | 10099 |
| ASRGLYYYDSSGYYFDYW | 306 | 39 | None | | 69628 |
| CALPI-FLMCCWIWFASILWKIFASMFITDVGLKFSF | 97 | 39 | None | | 69629 |
| FVVSLPGF | | | | | |
| AAQLLFYFDYW | 239 | 39 | None | | 69630 |
| CALPI--CFLTCCWIWFASILLRIFASMFIRDIRLKLS | 98 | 39 | None | | 69631 |
| FFVVTLPGF | | | | | |
| CARLDDYDSSGYFVDYW | 241 | 39 | None | | 25970 |
| CARDNRYDSSGLDYW | 549 | 39 | None | | 2457 |
| CAKLPDYYDSSGYPGDGFDIW | 267 | 39 | None | | 4470 |
| REYYWNYW | 233 | 39 | None | | 69632 |
| CARGDYAGYYYYMDVW | 268 | 39 | None | | 1865 |
| ARSYYYDSSGPFDYW | 210 | 39 | None | | 69633 |
| CAKDGELYYDSSGYFNFDPW | 365 | 39 | None | | 3541 |
| CAKDIIAVAGPFDYW | 222 | 39 | None | | 2748 |
| CALPICFLMCCWIQSASILLRIFTLMFIRGIGLTFSF | 97 | 39 | None | | 69634 |
| FVVSLPGF | | | | | |
| CAKDGRSSGWPYYFDYW | 749 | 39 | None | | 11273 |
| CSGGSCYGRPFDYW | 170 | 39 | None | | 69635 |
| CAKAYGG----YHFDYW | 646 | 39 | None | | 11279 |
| CARGGHSSGYYFDYW | 872 | 39 | None | | 27619 |
| CASLYYYDSSRLPFDYW | 179 | 38 | None | | 4516 |
| CARDLPIYCSGGSCY--VDYW | 122 | 38 | None | | 6483 |
| CAHRSGYSSGWYNWFDPW | 191 | 38 | None | | 6754 |
| CARG---YQGFDAFDIW | 326 | 38 | None | | 2345 |
| SPGGYYYDSSGYPIDYFDYW | 406 | 38 | None | | 69636 |
| CSSDLSFLMCCWIRFASIL-RIFASMFSKDIGLKFSFL | 93 | 38 | None | | 69637 |
| VVSLPGF | | | | | |
| CARGHSSGRYNWFDPW | 240 | 38 | None | | 11403 |
| CAKGIAVAGTCDYW | 243 | 38 | None | | 4947 |
| RYCSGGSCYRPW-AFDIW | 156 | 38 | None | | 69638 |
| CARDGGTYDSSGYYYGLDYW | 507 | 38 | None | | 10755 |
| CARDAALYYYDSSGYH-WFDPW | 264 | 37 | None | | 16529 |
| CARQGSDYYDSSG--DYW | 244 | 37 | None | | 1652 |
| CAKEGGYQDAFDIW | 197 | 37 | None | | 24178 |
| CATPMGPYYYDSSGYGADAFDIW | 273 | 37 | None | | 25750 |
| CARIYYGTNAFDIW | 216 | 37 | None | | 24787 |
| CARDTHYYDNSGYYYPRFGYW | 497 | 37 | None | | 5256 |
| GASTNNWFDPW | 269 | 37 | None | | 69639 |
| CAAWWELQDAFDIW | 135 | 37 | None | | 4630 |
| CAKAARGYCSGGSCYFGY | 147 | 37 | None | | 69640 |
| PSSGWTLYYFDYW | 155 | 37 | None | | 69641 |
| CARRGYCSGGSCYVPFEYW | 128 | 37 | None | | 4359 |
| CAKDHYYDSSGYPSPFDPW | 227 | 37 | None | | 1150 |
| CVKDRGSYHYYYYMDVW | 166 | 37 | None | | 5340 |
| CARGR-QYCSGGSCYDAFDIW | 129 | 36 | None | | 27268 |
| CARSSSSWSGYYYYGMDVW | 138 | 36 | None | | 28014 |
| CARDESSGY | 168 | 36 | None | | 69642 |
| CAYDYGGNLFDYW | 183 | 36 | None | | 6507 |
| CARDIERITIFGV--VDYW | 235 | 36 | None | | 27717 |
| ASYWNYNFDYW | 240 | 36 | None | | 69643 |
| CARRGAHFYDSSGYYPFDYW | 1715 | 36 | None | | 11026 |
| CARDAAFYYFD | 294 | 36 | None | | 69644 |
| CARVLASGYFDYW | 315 | 35 | None | | 27914 |
| CAKDISSGWYVHYFDYW | 185 | 35 | None | | 3885 |
| CARDSIAVAGTNYYYGMDVW | 145 | 35 | None | | 26162 |
| CARDRGSSSGYYYYMDVW | 189 | 35 | None | | 11459 |
| CAKDR---YDSSGYILLDAFDIW | 175 | 35 | None | | 24408 |
| CATDRG-SSGWPFDYW | 290 | 35 | None | | 4027 |
| AQTNYYYYYYMDVW | 395 | 35 | None | | 69645 |
| CAVSTIFGVVTWFDPW | 282 | 35 | None | | 2423 |
| CAKVDSYYDFWSGPD | 204 | 35 | None | | 69646 |
| CASERGYDFWSG--SPFDYW | 241 | 35 | None | | 27394 |
| CARVRDQYYYDSSG-PFDIW | 206 | 35 | None | | 6557 |
| CAREVYLYYDSSGPIDYW | 177 | 35 | None | | 3340 |
| SSTSCYVGWFDPW | 133 | 34 | None | | 69647 |
| CASIAAAGAPTYYYYGMDVW | 267 | 34 | None | | 25406 |
| CAKVFIAVAGYFDYW | 168 | 34 | None | | 2585 |
| IAAAGTTP-FDYW | 187 | 34 | None | | 69648 |
| CARDTYSSGWYFGWFDPW | 257 | 34 | None | | 1888 |
| SSSWSDYW | 259 | 34 | None | | 69649 |
| SSGWYRPPYYFDYW | 203 | 34 | None | | 69650 |
| CARSYYDILTGYYSSFDYW | 315 | 34 | None | | 26270 |
| CARD-SAYYEFWSGYYYFDYW | 595 | 34 | None | | 10694 |
| GLTSGWLDYW | 270 | 34 | None | | 69651 |
| CAKGDGYLSYYFDYW | 255 | 34 | None | | 27951 |
| CARVYDSSGYYSGEGDYW | 172 | 33 | None | | 24991 |
| NGYSYGSHDAFDIW | 141 | 33 | None | | 69652 |
| CARVTGYYYDSSAYLDYW | 285 | 33 | None | | 26434 |
| ASLYSTSSPYFDYW | 1078 | 33 | None | | 69653 |
| CARASYYSSGWYAFDYW | 988 | 33 | None | | 3173 |
| CARVSHPSYYYDSSGYAIDYW | 263 | 33 | None | | 414 |
| CAKVDSSSWHFDYW | 224 | 33 | None | | 7151 |
| CARDESGYYDSSGYYYSAFDIW | 548 | 33 | None | | 4995 |
| CARPSSGYYLGYFDYW | 219 | 33 | None | | 3582 |
| CARDISSSWYPNWFDPW | 326 | 33 | None | | 2579 |
| CAHRRSGWLGGYFDYW | 164 | 33 | None | | 25562 |
| CAKDLGAVAGYFDYW | 293 | 32 | None | | 10004 |
| CQQRSGWPV | 2859 | 32 | None | | 69654 |
| ATTSPGSTDAFDIW | 143 | 32 | None | | 69655 |
| CATGANTIFGVVIIDAFDIW | 231 | 32 | None | | 1995 |
| CAR-GWASGWSYYFDYW | 724 | 32 | None | | 6976 |
| CAKDRSYYYYDSSGYYHFFDYW | 283 | 32 | None | | 26095 |
| CARVTGYYDSRADYW | 160 | 32 | None | | 4505 |
| CAKDRYSSSWNDAFDIW | 197 | 32 | None | | 6426 |
| CAS----YDILTGSLDYW | 150 | 32 | None | | 3670 |
| CAKSGKYYYDSSGYYLFDLW | 202 | 32 | None | | 7682 |
| CAKQADYYDSSGYSWSQDYW | 205 | 32 | None | | 7099 |
| CARALYDSSGF--GYW | 224 | 32 | None | | 11062 |
| CALPILSFLMCCCIRFASISLRIFALMFIGHIGLKFS | 72 | 32 | None | | 69656 |
| FFVLFMPGF | | | | | |
| CARHSGSTYYDSSGYHIDYW | 1698 | 31 | None | | 6418 |
| CAKGGYCSGGSCDNWFDPW | 241 | 31 | None | | 261 |
| CARGEETYYDFWSGYLIDYW | 166 | 31 | None | | 7157 |
| CANLPDSSGYRDNW | 156 | 31 | None | | 25405 |
| CARDP-TNCSGGSCYPGDYW | 99 | 31 | None | | 7090 |
| CARDPDTYYYDSSGYYYENYFDYW | 161 | 31 | None | | 2728 |
| CATTAPHCSSTSCYSNWFDPW | 108 | 31 | None | | 1248 |
| RGSGDYDYW | 729 | 31 | None | | 69657 |
| CATLNLDSSG-YYFDYW | 200 | 31 | None | | 4188 |
| CAREGSGWYRYYFDYW | 223 | 31 | None | | 5322 |
| CARD-TGIAAAGTFDYW | 357 | 30 | None | | 27597 |
| CARGYDVLTGYPDYW | 648 | 30 | None | | 2018 |
| DFWSGYQTVDYW | 617 | 30 | None | | 69658 |
| SKYGSYYDYW | 201 | 30 | None | | 69659 |
| CARDTSYYYDSSVGYFDYW | 1697 | 30 | None | | 23443 |
| CARGTYYYDSSAL-AFDIW | 115 | 30 | None | | 26522 |
| CARDNPYYDSSGYDVGPFDIW | 277 | 30 | None | | 1554 |
| CAR--STIFGVVYFDYW | 143 | 30 | None | | 26663 |
| CARPIGSGNYDAFDIW | 153 | 29 | None | | 27145 |
| ARLRRGNDAFDIW | 180 | 29 | None | | 69660 |
| CARDPDDYDSSGYYGSLDYW | 211 | 29 | None | | 27668 |
| CARASWYYDSSGYRDYYYYGMDVW | 1733 | 29 | None | | 24641 |
| CAKDKGLDDAFDIW | 197 | 29 | None | | 389 |
| ARLNLYFDLW | 217 | 29 | None | | 69661 |
| CARGGSYDILTGPFDYW | 650 | 29 | None | | 26207 |
| CAKVPGGGSYFDYW | 194 | 29 | None | | 23784 |
| CAREYDSSGPWFDYW | 195 | 29 | None | | 2965 |
| CAKDLYGSGSYYPSDYW | 180 | 29 | None | | 2866 |
| CASQGYSSFWY | 132 | 29 | None | | 69662 |
| CARTDYYDSSGFGTLDYW | 206 | 29 | None | | 685 |
| LLYDPFDYW | 146 | 29 | None | | 69663 |
| CAREYSSSWPNDYW | 258 | 28 | None | | 7104 |
| CARMRGSGSYDDAFDIW | 160 | 28 | None | | 10966 |
| CARRGDYGWYFDYW | 279 | 28 | None | | 7644 |
| CAHRPDISLYYFDYW | 222 | 28 | None | | 3979 |
| VVVPAACDYW | 159 | 28 | None | | 69664 |
| CARDPWHYYDSSGYYPDARFDYW | 177 | 28 | None | | 6558 |
| CAGSGNPGDYW | 175 | 28 | None | | 26841 |
| ARRYCPGGSCYDAFDIW | 122 | 28 | None | | 69665 |
| CAKAPLEYCSGGTCYPFDYW | 150 | 28 | None | | 2870 |
| ASRGYNYFDYW | 662 | 28 | None | | 69666 |
| CASLPDYGDYPFDYW | 289 | 28 | None | | 6395 |
| CATGPFRES-YYFDYW | 96 | 28 | None | | 27730 |
| SYGAISFDYW | 134 | 27 | None | | 69667 |
| CARDGGSRHYYDSSGLFDYW | 1741 | 27 | None | | 24660 |
| GRTPDYYYDSSGIFDYW | 173 | 27 | None | | 69668 |
| LRAEDTAVYYCARGYSIDYW | 130 | 27 | None | | 69669 |
| CARRGSGYRQYYFDYW | 705 | 27 | None | | 6237 |
| CARSPYYDILTGYYY-IDYW | 273 | 27 | None | | 27832 |
| CATLAAAGGDYW | 208 | 27 | None | | 4005 |
| CAKGNPYSSGWYAFDYW | 225 | 27 | None | | 718 |
| VKQSHGLDYFDYW | 181 | 27 | None | | 69670 |
| CQQRSIWPLAF | 2735 | 27 | None | | 4375 |
| RETYYYDSSGYFRVGAFDIW | 206 | 27 | None | | 69671 |
| CARDHCSGGFCYIDSW | 106 | 27 | None | | 24436 |
| MTPRDAFDIW | 115 | 27 | None | | 69672 |
| CAKVL-PYYDFWSGY--WDYYYYGMDVW | 107 | 26 | None | | 1178 |
| CARDGVCSSTSCYLYYYYGMDVW | 79 | 26 | None | | 23692 |
| RVLLWFGDAFDIW | 154 | 26 | None | | 69673 |
| CHVWDSST | 11503 | 26 | None | | 69674 |
| CARDYFDSSGNYYVDYW | 245 | 26 | None | | 27075 |
| CARDSSGGRADYYDSSGYFDYW | 1681 | 26 | None | | 27719 |
| CAKDRRIAVAGPFDYW | 207 | 26 | None | | 4385 |
| CAKVLAVAGTGDYW | 166 | 26 | None | | 7396 |
| CARGAYYYDSSVPGDYW | 1657 | 26 | None | | 25101 |
| CARDRQLWDYYYGMDVW | 168 | 26 | None | | 6622 |
| CARYSSGYYSTSFDYW | 116 | 25 | None | | 2476 |
| CARGYSSGWYTIHFDYW | 147 | 25 | None | | 7548 |
| CAK-ADWGGLYYFDYW | 146 | 25 | None | | 10447 |
| CASDYGGHSFDYW | 130 | 25 | None | | 25841 |
| CARTYSNYVYYFDYW | 200 | 25 | None | | 2167 |
| CAGQGTYYDFWSGYYTGINYFDYW | 91 | 25 | None | | 24782 |
| NILTGYYNFFDYW | 293 | 25 | None | | 69675 |
| CARGG--DSSGYYSWYFDLW | 120 | 25 | None | | 6920 |
| CARTGYMGSGWYYFDYW | 762 | 24 | None | | 1188 |
| CARGD-SSGWYGNPYYFDYW | 272 | 24 | None | | 7501 |
| CAHRLYYDFWSGYYNGGVFDYW | 124 | 24 | None | | 11095 |
| CAKLRSGSYLSYFDYW | 140 | 24 | None | | 23563 |
| CAHRSSGWTPGWFDPW | 98 | 24 | None | | 1403 |
| CAKDITDGYCSGGSCSPGFDYW | 153 | 24 | None | | 1312 |
| YCSGGSCFPRLFDYW | 111 | 24 | None | | 69676 |
| CAR----GGTASGFDYW | 123 | 24 | None | | 7546 |
| CASFPRYDFWSGSSSDYW | 178 | 24 | None | | 2452 |
| CAKLPLRN--YYDSSGSEFDYW | 125 | 24 | None | | 3629 |
| CARLGGSSGYYQYYFDYW | 121 | 24 | None | | 25933 |
| RPTLGYCSGGSCY-RLAWFDPW | 121 | 24 | None | | 69677 |
| LYSSSWYQFDYW | 110 | 24 | None | | 69678 |
| CAAGG-DFWSGLDYW | 166 | 23 | None | | 24990 |
| CARDHPCSGGSCYSVRRFDPW | 120 | 23 | None | | 25372 |
| TYGSSSGWYSSFDYW | 87 | 23 | None | | 69679 |
| CARELVRGKYYYYYGMDVW | 544 | 23 | None | | 2534 |
| CAKDIGAVAAYFDYW | 141 | 23 | None | | 27172 |
| CARTHGYSYGTPYFDYW | 182 | 23 | None | | 2896 |
| CAKTIAVAGTNYFDYW | 123 | 23 | None | | 2679 |
| CAKDTGYSYGNDAFDIW | 141 | 23 | None | | 9793 |
| CARGLLGYCSGGSCYPAVFDYW | 127 | 23 | None | | 5145 |
| CARDGIAVAGFSFDYW | 215 | 23 | None | | 6357 |
| CARDPYYDFWSGL---YYYYYYMDVW | 68 | 22 | None | | 4444 |
| YGSGSYSSYYFDYW | 82 | 22 | None | | 69680 |
| CAAWDDSLNGPVF | 11218 | 22 | None | | 24 |
| ARMNCSGPSCYPWFDYW | 111 | 22 | None | | 69681 |
| CARIRGTIFGVVLDYW | 153 | 22 | None | | 26740 |
| CAKDARIYSSGWSPYYFDYW | 534 | 22 | | | 2339 |
| CAHRLSVAGPYFDYW | 130 | 22 | None | | 6449 |
| CARGSYGGMVFDYW | 595 | 22 | None | | 2279 |
| CAY--GGSNYKLTF | 73 | 21 | None | | 69682 |
| CARDPPYGSGSYYNPFDYW | 112 | 21 | None | | 2353 |
| CAAWDDSLSGWVF | 11220 | 21 | None | | 23 |
| CARDLGMGSYFDYW | 299 | 21 | None | | 5010 |
| RDLDFWSGYALWDYW | 100 | 21 | None | | 69683 |
| AEDYYDSSADYW | 114 | 21 | None | | 69684 |
| CARASYSSRDYYYYMDVW | 107 | 21 | None | | 2054 |
| CARNTGYSSSW-CDAFDIW | 113 | 21 | None | | 7047 |
| CSKDGGTYESPFDYW | 85 | 21 | None | | 475 |
| ARDGGFRPWFDPW | 109 | 21 | None | | 69685 |
| CAVWDDSLSGRVF | 11225 | 21 | None | | 22 |
| CAMTMNYDSSGYGFVYW | 84 | 21 | None | | 6457 |
| CAKAEGYTSGWY-FDYW | 298 | 20 | None | | 2674 |
| CARVVIR-VGFDYW | 214 | 20 | None | | 5164 |
| CAAWDDSLSGPVF | 11212 | 20 | None | | 25 |
| CATKGYYSSSTDYW | 92 | 20 | None | | 24069 |
| CARGGIVDWYFDLW | 76 | 20 | None | | 24877 |
| GYSYGTALFDYW | 98 | 20 | None | | 69686 |
| CARGGGIGWGGYYFDYW | 226 | 20 | None | | 7558 |
| SGSFYSGDYW | 114 | 20 | None | | 69687 |
| CARERRSIAAAGYFDYW | 260 | 20 | None | | 27033 |
| CARTHYDILTGYLTIFDYW | 218 | 20 | None | | 6540 |
| CARDPGPKGA-FDIW | 549 | 19 | None | | 2183 |
| CARAAYCGGDCYSGHDYW | 111 | 19 | None | | 2586 |
| CAREVRSGSYYYYYSMDVW | 372 | 19 | None | | 856 |
| GGNEWGPFDYW | 164 | 19 | None | | 69688 |
| CARDIAVAGQYYFDYW | 431 | 19 | None | | 938 |
| CARNFNYDSSGLDYW | 80 | 19 | None | | 9751 |
| CARDRYGYYYDSSSNYFDYW | 152 | 18 | None | | 3942 |
| CAR--LGYYGSGTPGDYW | 77 | 18 | None | | 6695 |
| CARGLGGDRWFDPW | 146 | 18 | None | | 2477 |
| CARYGSGSYWSWYFDLW | 144 | 18 | None | | 2371 |
| CARAPGGYCSSTSCYGSYYYYGMDVW | 43 | 18 | None | | 3774 |
| CTTTVDPYDFWSGWAYFDYW | 444 | 18 | None | | 1465 |
| CTRDPAGYCSGGSCHYLHFDYW | 63 | 18 | None | | 23915 |
| CAKLTYGDYSGPDYW | 128 | 18 | None | | 1600 |
| YYDSESGDYW | 135 | 18 | None | | 69689 |
| SGGSYIPTF | 61 | 18 | None | | 69690 |
| CAAWNDSLSGPNWVF | 11199 | 17 | None | | 27 |
| CAAWDESLNGFCLF | 9466 | 17 | None | | 3761 |
| CAAWDDNLSGPVF | 11204 | 17 | None | | 26 |
| CAKVGSGSGWYADYW | 97 | 17 | None | | 27884 |
| CASDR-NYYDILTGSLDYW | 91 | 17 | None | | 1810 |
| CSSDLFLICCWIRLASILLRILVSMFIKDIGLQFSFL | 39 | 17 | None | | 69691 |
| VMSFSGF | | | | | |
| CARGGLGYSYGGWYFDYW | 121 | 17 | None | | 6279 |
| CASL---DSSWLPFDYW | 90 | 17 | None | | 713 |
| CAR--GLYSSSWRGFDIW | 57 | 17 | None | | 9840 |
| CATWDDSLNGYAVF | 11197 | 16 | None | | 28 |
| RQVPHGFDIW | 281 | 16 | None | | 69692 |
| CAAWDDSRNGPHVVF | 9463 | 16 | None | | 3350 |
| LAYCSTTSCSGTDYW | 73 | 16 | None | | 69693 |
| CQVWESSSDHRWVF | 659 | 16 | None | | 3886 |
| CARR--AAGRHYYYYYYMDVW | 112 | 16 | None | | 5364 |
| CATGYCSGGSCYHAKTPDYW | 93 | 16 | None | | 25787 |
| CALRSGGYQKVTF | 59 | 16 | None | | 69694 |
| CAAWDDSLNGHVVF | 11197 | 16 | None | | 29 |
| CATAPGIAVAGTRWFDPW | 73 | 16 | None | | 762 |
| GGSYERGNWFDPW | 264 | 16 | None | | 69695 |
| CARGGDSSSWHNNWFDPW | 75 | 15 | None | | 6462 |
| CARHQSGYSYGRFDYW | 91 | 15 | None | | 6803 |
| CAVWDDSLPGRWLF | 3174 | 15 | None | | 10551 |
| CAAPSGGYQKVTF | 58 | 15 | None | | 69696 |
| CATWDDTLSGLNWVF | 11194 | 15 | None | | 30 |
| CARDLRRGYSYGAIDYW | 91 | 15 | None | | 1565 |
| CATHSGGYQKVTF | 57 | 15 | None | | 69697 |
| CIVRSGGYQKVTF | 57 | 15 | None | | 69698 |
| HGSGSYYNPVDYW | 80 | 14 | None | | 69699 |
| CAFLSGGSNYKLTF | 57 | 14 | None | | 69700 |
| TIFGVVIVVGWFDPW | 64 | 14 | None | | 69701 |
| AGGLVRASSFDYW | 126 | 14 | None | | 69702 |
| VYGDDGDYW | 54 | 14 | None | | 69703 |
| RCSGGSCLYYDSW | 56 | 14 | None | | 69704 |
| CARLTG-GRPSDAFDIW | 86 | 14 | None | | 26012 |
| CGTWDSSLSAVVF | 5058 | 14 | None | | 9776 |
| CARAREDIVVVPAATHYYYYGMDVW | 79 | 14 | None | | 593 |
| CARDHWGSFEY | 47 | 13 | None | | 69705 |
| CVHPSGTYKYIF | 71 | 13 | None | | 69706 |
| CQQYMHWP | 857 | 13 | None | | 69707 |
| CAKDEKIAAAGTGSFDYW | 65 | 13 | None | | 10643 |
| AAAGTIDWFDPW | 83 | 13 | None | | 69708 |
| VASSANAFDIW | 49 | 13 | None | | 69709 |
| CGTLSGGYQKVTF | 53 | 13 | None | | 69710 |
| CARDLGGGTLGDYW | 178 | 13 | None | | 7697 |
| CAKGYSGSYYGAVDYW | 27 | 12 | None | | 4200 |
| CMQGIHWPPTF | 4058 | 12 | None | | 27735 |
| CSSDLSQLQLTTGNGLFLSEGLKLVDKFLEDVKKLYHS | 74 | 12 | None | | 69711 |
| EAFTVNF | | | | | |
| RDSPGAHFDYW | 59 | 12 | None | | 69712 |
| CGTWDSSLSVYVF | 5023 | 12 | None | | 4688 |
| CARDGSAYYAPNWFDPW | 63 | 12 | None | | 27711 |
| CALPI-SFEKKAVIKDLEFPVAFEPPLKGIMAAASRLS | 47 | 11 | None | | 69713 |
| CGSKRKSF | | | | | |
| CALPISGCWAFLFIQTVLPSLQGPLTPDGHRVPPADH | 288 | 11 | None | | 69714 |
| RAWLSPEGGF | | | | | |
| CARYGYGERGFDSW | 43 | 11 | None | | 24146 |
| CSSDLFEKKAVIKDLEFPVAFEPPLKGIMAAASRLSC | 47 | 11 | None | | 69715 |
| GSKRKSF | | | | | |
| CALPIFWASLFNQTVFLGLQGPLTPDGHRVPPADHRA | 288 | 11 | None | | 69716 |
| WLSPEGGF | | | | | |
| CALPI-WASLFNQTVFLGLQGPLTPDGHRVPPADHRAWL | 288 | 11 | None | | 69717 |
| SPEGGF | | | | | |
| CALPICAFEKKAVIKDLEFPVAFEPPLKGIMAAASRLS | 47 | 11 | None | | 69718 |
| CGSKRKSF | | | | | |
| CALPIFQIQTSSLESCIYMWLDKYLEEKNTKRDSPHS | 72 | 9 | None | | 69719 |
| LPPPTIFFNF | | | | | |
| CQVWDIISDHQGVF | 594 | 9 | None | | 27623 |
| DLQTSSLESCIYMWLDKYLKEKNTKRDSPHSLPPPTI | 72 | 9 | None | | 69720 |
| FFNF | | | | | |
| SKLCDVTEHIESVVIVVLLSSLSPVSCYTYSVPNCKLNF | 60 | 9 | None | | 69721 |
| CALPI--CKARLCKVSPLQTEWFKTARMHSSIYNSKAP | 42 | 9 | None | | 69722 |
| CTELGF | | | | | |
| CALPIW-PCKARLCKVSPLQTEWFKTARMHSSIYNSKA | 42 | 9 | None | | 69723 |
| PCTELGF | | | | | |
| CASSTGQGYEQYF | 48 | 9 | None | | 69724 |
| CYSAADNNRVF | 774 | 9 | None | | 1598 |
| CALPISRVVWRPCKARLCKVSPLQTEWFKTARMHSSI | 42 | 9 | None | | 69725 |
| YNSKAPCTELGF | | | | | |
| CATWDG-YYKKLF | 394 | 9 | None | | 69726 |
| CLLTVSFNLSFGNMEGKRINVSKMKLRLKSSLPRKLD | 51 | 8 | None | | 69727 |
| KSNFKAMVSTF | | | | | |
| CALPIWKHEERQLGDCQRQVSIRIWPGPVLHLGKRIF | 32 | 8 | None | | 69728 |
| SSCFYYFTF | | | | | |
| CYSAADNNLRVF | 771 | 8 | None | | 5359 |
| CALPIY--EERQLGDCQRQVSIRIWPGPVLHLGKRIF | 32 | 8 | None | | 69729 |
| SSCFYYFTF | | | | | |
| CATWDG-YYKKLF 394 9 None None None | | | | | 69726 |

TABLE A5
CDR3 Alignements Exclusive to Non-Survivors Day 0

| | Cluster | CDR3 | | | |
|---|---|---|---|---|---|
| Alignment | Counts | Counts | Class | ID | SEQ ID NO |
| CQQSYSTPLFAF | 28 | 140569 | C4 | EY6A | 34 |
| CQQYYTTPMYAF | 23 | 125715 | C4 | CR3022 | 35 |
| CQQYYSTPPSF | 34 | 110321 | C4 | CR3022 | 36 |
| CQSADSSGTWVF | 37 | 62077 | C4 | SZA4 | 37 |
| CQSADTSGTYGNWVF | 22 | 61809 | C3 | S309 | 38 |
| CQSADTSGPYRDVVF | 17 | 61626 | C3 | S309 | 39 |
| CQSPDSSATYQV | 11 | 61577 | C4 | SZA4 | 40 |
| CQSADNSGSYGIF | 22 | 51193 | C4 | SZA4 | 41 |
| CQQYGSSVTF | 27 | 48316 | C1 | C102 | 42 |
| CQSADSSGTYKMLF | 16 | 30329 | C4 | SZA4 | 43 |
| CQRYNNYPYIF | 15 | 25022 | C3 | C110 | 44 |
| CQRYNTAPYTF | 12 | 22628 | C3 | C110 | 45 |
| CQQVYDTPRTF | 12 | 19935 | C2 | C002 | 46 |
| CMQGTHWPPIIF | 19 | 14254 | C1 | B38 | 47 |
| CIQGTHWPPSAF | 14 | 14235 | C1 | B38 | 48 |
| CMQGTHWPPSTF | 16 | 14233 | C1 | B38 | 49 |
| CMQGTHWPPAF | 20 | 14141 | C1 | B38 | 50 |
| CMQGTLWPPSF | 13 | 14091 | C1 | B38 | 51 |
| CMQGTHWPPGLTF | 13 | 14077 | C1 | B38 | 52 |
| CMQGTHWPPSFTF | 14 | 14076 | C1 | B38 | 53 |
| CMQGTHWPPTWTF | 13 | 14073 | C1 | B38 | 594 |
| CQAYDSSLV | 23 | 12282 | C4 | S2A4 | 69730 |
| CQAWDSSTAVF | 19 | 12245 | C4 | SZA4 | 23352 |
| CQAWDSSTVVF | 18 | 12215 | C4 | SZA4 | 3867 |
| CHSRDSSNNHH-FLF | 14 | 9606 | C4 | SZA4 | 13855 |
| CSSRDSSGDHR | 12 | 8852 | C4 | SZA4 | 69731 |
| CQQRSNWPPFT | 22 | 7867 | C3 | REGN10987 | 69732 |
| CSSRDSSGNHRLF | 18 | 7447 | C4 | SZA4 | 23203 |
| CYSTDGSGN | 12 | 7445 | C1 | C105 | 69733 |
| CHSRDSSANHLGYVF | 15 | 7433 | C4 | SZA4 | 9074 |
| CHSRDSSGNHRGFVF | 14 | 7432 | C4 | SZA4 | 5470 |
| CQQFNSYLF | 34 | 5415 | C1 | B38 | 5417 |
| CSSYAGSNNWVF | 11 | 5256 | C2 | P2B-2F6 | 18923 |
| CSSHAGSNNLLF | 10 | 5068 | C2 | P2B-2F6 | 23163 |
| CQTWGTGIQVF | 7 | 4772 | C2 | C104 | 5654 |
| CQTWGTGIWVF | 7 | 4772 | C2 | C104 | 7863 |
| CQQYGSSSIIF | 10 | 3662 | C1 | C102 | 22947 |
| CCSYAGSYPFNWVF | 10 | 3473 | C2 | COVA2-39 | 23401 |
| CQHYNNWPSYTF | 21 | 3437 | C3 | REGN10987 | 22000 |
| CQPYNNWPQYTF | 14 | 2979 | C3 | REGN10987 | 5640 |
| CQSYDASLGGSGLF | 16 | 2646 | C4 | SZA4 | 21229 |
| CQQYDNLLLLTF | 14 | 2454 | C3 | REGN10987 | 5899 |
| CQQYDNLLALTF | 9 | 2417 | C3 | REGN10987 | 16588 |
| CQSYDNSLGGWKLF | 16 | 2206 | C4 | SZA4 | 5562 |
| CQQYDNLLLFTF | 14 | 2198 | C3 | REGN10987 | 21706 |
| CQQYDNLLSITF | 10 | 2142 | C3 | REGN10987 | 19542 |
| CQSYDRSLNIWVF | 11 | 1853 | C3 | REGN10987 | 5466 |
| CCSYAGSYSF | 11 | 1694 | C2 | COVA2-39 | 16865 |
| CCSYAGSYTFDWVF | 10 | 1692 | C2 | COVA2-39 | 6155 |
| CCSYAGSYTFLNWVF | 9 | 1688 | C2 | P2B-2F6 | 14171 |
| CSYAGSYTFVWVF | 9 | 1688 | C2 | COVA2-39 | 18735 |
| CQYYNNYSTF | 15 | 1164 | C3 | C135 | 8511 |
| CASLYGGNSYYFDYW | 91 | 895 | C1 | C105 | 18817 |
| CATAPRYYYDSSGYWGPNDYW | 83 | 864 | C1 | C105 | 19448 |
| CYQYGNSPP | 11 | 853 | C1 | C102 | 69734 |
| CQVWHSTSDHSVVF | 8 | 744 | C4 | CR3022 | 13565 |
| CYHFDYW | 3630 | 500 | None | | 19582 |
| CARYHYDSSGHFDYW | 61 | 412 | C4 | S2A4 | 9351 |
| CATNDAFDIW | 3003 | 393 | None | | 9187 |
| CAYQNSSGWFGYYFDYW | 35 | 349 | C2 | COVA2-39 | 18984 |
| GLGSYYFDYW | 4054 | 310 | None | | 69735 |
| CAKDLGDYYDSSGHNWFDPW | 33 | 287 | C4 | SZA4 | 14468 |
| TGSSGWYYFDYW | 2900 | 252 | None | | 69736 |
| GRFYYYYYMEVW | 1334 | 233 | None | | 69737 |
| CATYYYDSSGYYLDFDYW | 2313 | 214 | None | | 17363 |
| CSSTSCSDNWFDPW | 29 | 211 | C1 | C105 | 69738 |
| CQVWDSSNDHPVF | 15 | 202 | C4 | SZA4 | 6196 |
| CAHSKSSAWYQIDY | 27 | 194 | C2 | COVA2-39 | 69739 |
| GGNPNYYYYGMDVW | 1239 | 185 | None | | 69740 |
| CQVVDSSSDLF | 9 | 184 | C4 | SZA4 | 8595 |
| CTRYSVSYYYYGMDVW | 1425 | 183 | None | | 13140 |
| AQIDYYYYMDVW | 1430 | 181 | None | | 69741 |
| CTIGTNWFDPW | 1580 | 177 | None | | 5691 |
| CAYNSGGNYYYYGMDVW | 1298 | 175 | None | | 21352 |
| CKTDYYDSSGYPGYFDYW | 1656 | 167 | None | | 16875 |
| CASGSYPYYFDYW | 1369 | 167 | None | | 5444 |
| CARYYSPYYYYAMDVW | 1300 | 164 | None | | 23041 |
| ATYDNSGYFDYW | 1756 | 161 | None | | 69742 |
| CATSYYYDSSGYLDYW | 1439 | 161 | None | | 6106 |
| CARGFDYW | 2571 | 161 | None | | 16640 |
| CQQYENFPVAF | 13 | 160 | C3 | REGN10987 | 20246 |
| GTSAYYYYYMDVW | 1109 | 153 | None | | 69743 |
| AKGTDWYYYYGMDVW | 964 | 151 | None | | 69744 |
| GVDYYYMDVW | 1222 | 145 | None | | 69745 |
| CARDGYYDSSGYFDYW | 2310 | 143 | None | | 20035 |
| CVRGN--YNFDYW | 1724 | 140 | None | | 14151 |
| TTQYYYDSSGYYYGDYW | 1263 | 140 | None | | 69746 |
| VSYDSSGYHRRFDYW | 1360 | 139 | None | | 69747 |
| GSGSYYTPFDYW | 1043 | 137 | None | | 69748 |
| CARSGYYYDSSGYPFDYW | 1349 | 136 | None | | 22262 |
| CTTDLGYYDSSGYRFDYW | 1938 | 132 | None | | 13804 |
| CARDGYDFWSGYQDYW | 1299 | 131 | None | | 19352 |
| CSGGSCHSFDYW | 1478 | 130 | None | | 19509 |
| CAKDLYPYYYGMDVW | 986 | 126 | None | | 11931 |
| TTDSSGYFAFDIW | 1093 | 123 | None | | 69749 |
| CAIWEYYYDSSGYTDYW | 1010 | 123 | None | | 11951 |
| TGDGNWFDPW | 813 | 120 | None | | 69750 |
| CSGGSCYRDDYW | 1279 | 120 | None | | 21859 |
| CASGSGYASDAFDIW | 1094 | 120 | None | | 8344 |
| CAKGGSGWFDPW | 873 | 119 | None | | 17495 |
| CASYS-SGWYSPFDYW | 1516 | 119 | None | | 5767 |
| CARDRYYYDSSGYYYGFDYW | 1626 | 118 | None | | 22200 |
| CTSFIYYYDSSGYYSDAFDIW | 1248 | 115 | None | | 8867 |
| CARGYCSGGSCYPHFDYW | 1130 | 112 | None | | 21535 |
| CSGGSCYGDYYYYGMDVW | 627 | 112 | None | | 20016 |
| AEDYYYYFMDVW | 656 | 112 | None | | 69751 |
| CAMYDTSGSFDYW | 1119 | 111 | None | | 22341 |
| CARD---YYDSSGYNDAFDIW | 1954 | 110 | None | | 12798 |
| TLGGYYYDSSGYYVYW | 881 | 110 | None | | 69752 |
| CSGGSCYSIYYFDYW | 1219 | 110 | None | | 15407 |
| CFSTGYLDYW | 995 | 106 | None | | 14223 |
| CATYVAYYYDSSGYRAFDYW | 901 | 106 | None | | 12114 |
| CAAAAAGTSYYYGMDVW | 1189 | 106 | None | | 22031 |
| CAAGRGSENYYYGMDVW | 890 | 104 | None | | 20914 |
| CAAPRGYDSSGY-DAFDIW | 1447 | 104 | None | | 17311 |
| CARGLDAFDVW | 892 | 103 | None | | 20654 |
| CVSSSGYSGWFDPW | 1105 | 103 | None | | 17493 |
| CATTYYYDSSGVLDYW | 785 | 101 | None | | 15574 |
| CATGRDYYDSSGYRIFDYW | 1221 | 100 | None | | 19077 |
| SSGWYNYYYYGMDVW | 613 | 100 | None | | 69753 |
| CARKYCSGGSCYFDYW | 1024 | 100 | None | | 15451 |
| CAKDTYDSSGY | 1293 | 100 | None | | 69754 |
| CARDLQDYYDSSGYYDYW | 1613 | 99 | None | | 22030 |
| CAKADYYDSSG--DYW | 1482 | 98 | None | | 23268 |
| CAHR-GDFWSGYFDYW | 1105 | 97 | None | | 16846 |
| CATVSFPNYYDSSGYY-YFDYW | 1071 | 97 | None | | 17069 |
| CATLHYYDSSGYYRPDAFDIW | 969 | 97 | None | | 14959 |
| ATISGYPVDYW | 1001 | 96 | None | | 69755 |
| CATRYCSGGSCYGYYFDYW | 1087 | 96 | None | | 23298 |
| SSGWYLGWFDPW | 1051 | 95 | None | | 69756 |
| CQICDFWSGYYSDYW | 938 | 93 | None | | 15358 |
| YYDSSGYHPLPFDYW | 806 | 93 | None | | 69757 |
| CAKALGYYYDSSGYSPFDYW | 1059 | 93 | None | | 12562 |
| CARGGYCSGGSCSPDDYW | 836 | 92 | None | | 7811 |
| AKAGTRYYFDYW | 691 | 92 | None | | 69758 |
| CARDLYYDSSGFDYW | 1264 | 92 | None | | 16936 |
| CSSASCYYYFDYW | 654 | 92 | None | | 18937 |
| CARGYSSSWYVYYW | 853 | 90 | None | | 22319 |
| CARAI--DYYDSSGYYPDAFDIW | 1094 | 87 | None | | 16085 |
| CTTGGTHDYYYYYMDVW | 691 | 86 | None | | 18361 |
| CARGYCSGGSCYSGGSDYW | 808 | 86 | None | | 17857 |
| CAGFEYSSSP-GYYYYGMDVW | 595 | 85 | None | | 14523 |
| IAGDYYYMDVW | 1050 | 84 | None | | 69759 |
| DCSGGSCYPDYW | 558 | 84 | None | | 69760 |
| RHNAFEIW | 718 | 84 | None | | 69761 |
| CAKVSGYHEDAFDIW | 935 | 84 | None | | 14777 |
| VTGPKYYFDSW | 486 | 84 | None | | 69762 |
| LSRYSSSWYYFDYW | 1350 | 84 | None | | 69763 |
| CASLAYYYDSSGYYSSAFDIW | 1077 | 83 | None | | 14389 |
| VGYCSGGSCYSGAFDIW | 822 | 83 | None | | 69764 |
| CAR---SGYSLFDYW | 1032 | 82 | None | | 5400 |
| RGYNDFDYW | 620 | 82 | None | | 69765 |
| CAR--GPYYDILTGYYPNDYW | 999 | 81 | None | | 21736 |
| CTRGTYYYDSSGYPNDAFDIW | 916 | 81 | None | | 17355 |
| CVRDLYYDSSGYFLSW | 1647 | 81 | None | | 5882 |
| CAKGDCSGGSCYDY | 915 | 81 | None | | 69766 |
| CARDLYCSGGSCYSSGFDYW | 1027 | 80 | None | | 20870 |
| CAKDSGYYDFWSGYSYFDYW | 791 | 80 | None | | 19317 |
| TTLYEAYYFDYW | 672 | 80 | None | | 69767 |
| CASIRYCSSTSCYPFDYW | 524 | 80 | None | | 18914 |
| CARDGSGYYDSSGYLDYW | 1629 | 79 | None | | 15369 |
| CTTTTYYYDSSGYSNDAFDIW | 1336 | 79 | None | | 14855 |
| CARGGYCSGGSCYSIYYFDYW | 781 | 78 | None | | 12058 |
| CARED---GSQYYYYGMDVW | 586 | 78 | None | | 14016 |
| APAATEGWFDPW | 576 | 78 | None | | 69768 |
| CARDRARYYYDSSGYPLPDYW | 830 | 78 | None | | 13287 |
| CAKD---TGYCSGGSCYGTFDYW | 602 | 78 | None | | 15763 |
| CARDPGYCSSTSCYEPFDYW | 525 | 78 | None | | 21753 |
| CARHGDPYYYDSSGYFDYW | 909 | 78 | None | | 21824 |
| CARDGDGYCSGGSCYK--FDYW | 951 | 77 | None | | 14407 |
| CTTDDSGYDSPFDYW | 961 | 76 | None | | 8932 |
| CARDGQGVSSYYYYYGMDVW | 659 | 76 | None | | 20535 |
| CAR-YSSGWFDAFDIW | 1171 | 75 | None | | 15595 |
| HLYYDSFGGFDYW | 1555 | 73 | None | | 69769 |
| CASDRSTYYYDSSGYDYW | 1002 | 73 | None | | 8446 |
| GHFHYYSAMDVW | 486 | 72 | None | | 69770 |
| CARGVPYDSSGYSLDAFDIW | 520 | 72 | None | | 15178 |
| GSGTHDYW | 1073 | 72 | None | | 69771 |
| GTYGDWAFDIW | 916 | 72 | None | | 69772 |
| CATRGYSDAYFDYW | 650 | 72 | None | | 15176 |
| CATPRGYSSGPFDYW | 957 | 71 | None | | 22183 |
| CAKDLTYYYDSSGQN | 1264 | 71 | None | | 69773 |
| CARVGKYCSGGSCYRTFDYW | 850 | 69 | None | | 21649 |
| CARDQDPYYYDSSGYYDAFDIW | 1477 | 69 | None | | 16432 |
| CAKD---FQGDYYYYGMDVW | 496 | 69 | None | | 5465 |
| CARLGGTYPDAFDIW | 686 | 68 | None | | 22226 |
| CAKD--SSGYSVRFDYW | 572 | 68 | None | | 8288 |
| CSSTSCSPNWFDPW | 480 | 68 | None | | 12462 |
| CASL--EGGYCSGGSCY--FDYW | 892 | 68 | None | | 20338 |
| CAKGLGYCSGGSCYSDEFDYW | 532 | 68 | None | | 20046 |
| CAKDRCSSTSCYYYYGMDVW | 374 | 68 | None | | 23343 |
| SSGYYPASDYW | 458 | 67 | None | | 69774 |
| CARDSDYYDSSGYLAPGDYW | 921 | 67 | None | | 5953 |
| CALGDCSGGSCYSDAFDIW | 857 | 67 | None | | 16187 |
| CARLNSGFDYYYGMDVW | 486 | 67 | None | | 14287 |
| CARDGDYYDSSGSPYYFDYW | 861 | 67 | None | | 8097 |
| YCSGGRCYAFDYW | 640 | 66 | None | | 69775 |
| CVPNYCSSTSCYDYW | 442 | 65 | None | | 20287 |
| CARHGCSGGSCYSFDSW | 738 | 65 | None | | 23141 |
| CARAGNYYYS | 758 | 64 | None | | 69776 |
| CANLAVAGDYYYHGMDVW | 796 | 64 | None | | 11892 |
| GSCSGGSCYSFDYW | 622 | 64 | None | | 69777 |
| TEYSSSSPYFDYW | 531 | 63 | None | | 69778 |
| CARAPVYYYDSSGYYSHFDYW | 719 | 63 | None | | 17317 |
| CASEDKYCSSTSCYGFDYW | 470 | 62 | None | | 8050 |
| CARDLTGTGEDYYYYGMDVW | 435 | 62 | None | | 12282 |
| CAR--VECSGGSCYSIHFDYW | 823 | 62 | None | | 23131 |
| CARQPLLYYYDSSGYYPFDYW | 627 | 62 | None | | 22785 |
| CAKEPSGY-YYYHMDVW | 388 | 61 | None | | 8465 |
| CARDLSGWYGHYYYYGMDVW | 457 | 61 | None | | 12518 |
| CARDTQRSSGSYYYYGMDVW | 543 | 61 | None | | 21217 |
| CARDSWYYDFWSGYHDAFDIW | 482 | 61 | None | | 16102 |
| CARDSRYCSGGSCY--SIWFDPW | 611 | 61 | None | | 15919 |
| CAK-TSSGWYADAFDIW | 777 | 61 | None | | 19730 |
| CAKLPGDYYDSSGY-YMVDYW | 501 | 61 | None | | 14452 |
| CAKD--VCSGGSCYEGAFDYW | 792 | 61 | None | | 20116 |
| SFGSGSYYYYALDVW | 475 | 59 | None | | 69779 |
| CASLKGYSSGWYSYYYGMDVW | 396 | 58 | None | | 21534 |
| CATGPDYYDSSG-FYYW | 440 | 58 | None | | 5376 |
| VVVVPATPGYYYYGMDVW | 411 | 58 | None | | 69780 |
| CARGRDCSGGSCYSPWFDPW | 785 | 58 | None | | 8200 |
| VMTTGRYNWFDPW | 270 | 57 | None | | 69781 |
| CARGSRS-GQGYYYYGMDVW | 518 | 57 | None | | 21092 |
| TTESGWYGVYFDYW | 615 | 57 | None | | 69782 |
| AGKSCSSTSCYSDFDYW | 570 | 57 | None | | 69783 |
| CARDLDYYDSSGYYYEGFDYW | 1480 | 57 | None | | 16702 |
| CARDRPSYDSSGYYLDYW | 334 | 57 | None | | 16452 |
| CARVFPTY-YDILTGYSTDYW | 463 | 56 | None | | 20117 |
| CARDGHYYYDSSGYRNDAFDIW | 1276 | 56 | None | | 14018 |
| CARAGYCSSTSCYSMDYW | 377 | 56 | None | | 21929 |
| CARQVGYCSGGSCSYDFDYW | 679 | 56 | None | | 19879 |
| GPDYYDSSGPFDYW | 882 | 56 | None | | 69784 |
| DSSGYPVGDYW | 683 | 56 | None | | 69785 |
| CARDDSGWFDPW | 467 | 55 | None | | 5411 |
| CARHGLYCSGGSCY --- NWFDPW | 647 | 55 | None | | 6049 |
| DYGDNQFDYW | 864 | 55 | None | | 69786 |
| CARLRPCSGGSCYSSPPDYW | 555 | 55 | None | | 21115 |
| CASPHSSGWHLFDYW | 601 | 53 | None | | 16655 |
| CARYGSGYYYDDAFDIW | 314 | 53 | None | | 18697 |
| CARTHLYCSGGSCYLPPFDYW | 527 | 53 | None | | 20778 |
| CAKDLGGY | 394 | 53 | None | | 69787 |
| CARAYCSSTSCYKYFDYW | 397 | 51 | None | | 14845 |
| CATVPGAPYYFDYW | 555 | 51 | None | | 8708 |
| CASKGGHCSSTSCYAFDYW | 293 | 51 | None | | 17679 |
| CARDL--CSGGSCYNIDYW | 487 | 51 | None | | 9433 |
| CANFGDYYDSSGTDDYW | 396 | 50 | None | | 16206 |
| DSSGLCDYW | 498 | 50 | None | | 69788 |
| GSGSYNRPFDYW | 497 | 50 | None | | 69789 |
| CATESGATNAFDIW | 910 | 49 | None | | 20325 |
| CAKGPGEYYDSSGYQYYFDYW | 393 | 49 | None | | 14012 |
| CARVVTPVYFDYW | 329 | 49 | None | | 17671 |
| CARSPLDCSGGSCYSRRFDYW | 538 | 49 | None | | 11858 |
| CARLGDSSGYVFDYW | 592 | 49 | None | | 8418 |
| CARHEGSYYDILTGYYPGDYW | 627 | 49 | None | | 6014 |
| CARVLANHYYYYYYMDVW | 227 | 49 | None | | 8491 |
| YDILTGYVYYYSMDVW | 330 | 49 | None | | 69790 |
| ARRGYYEYYFDSW | 268 | 49 | None | | 69791 |
| CARAVTYYYDSSG--NDAFDIW | 1139 | 48 | None | | 15599 |
| CARDGSGYSFHYW | 362 | 47 | None | | 7816 |
| CATPRSPYYYDSSGPLDYW | 446 | 47 | None | | 13466 |
| CARRGGRTSLYYYYYGMDVW | 483 | 47 | None | | 19182 |
| TGRDGYNYAFDYW | 305 | 47 | None | | 69792 |
| CARAGYCSGGSCSSVYYYYGMDVW | 273 | 47 | None | | 17861 |
| CASGYCSGGSCYYHYYYYMDVW | 310 | 47 | None | | 12333 |
| CARAYCGGDCYS---GAFDIW | 409 | 47 | None | | 8063 |
| CARHG-YYFDSSGYYEDAFDIW | 771 | 46 | None | | 19964 |
| ASWGGNSFDYW | 367 | 45 | None | | 69793 |
| CARIPEAYYYDSSGYWFDYW | 596 | 45 | None | | 17022 |
| CAKDRSHYYYDSSGFDYW | 833 | 45 | None | | 12636 |
| CAREDCSGGSCYSVGWFDPW | 329 | 45 | None | | 18438 |
| CATVLPPSYYDSSGYYYWFDYW | 574 | 45 | None | | 5762 |
| TGLRGWFDPW | 319 | 44 | None | | 69794 |
| CANDGPGYYD | 699 | 44 | None | | 69795 |
| CARTRYSSSYYFDYW | 779 | 44 | None | | 20769 |
| CARDRLYFDT | 914 | 44 | None | | 69796 |
| CARRGSAYYDILTGYYPYYFDYW | 241 | 44 | None | | 14390 |
| ARLDYYDSTGKFDYW | 797 | 44 | None | | 69797 |
| CARESSGWSNFDYW | 659 | 43 | None | | 9483 |
| CARDTPAYCSSTSCYYYYYGMDVW | 247 | 43 | None | | 21122 |
| CAREWHCSGGSCYS--FGYW | 597 | 43 | None | | 18147 |
| CATGGYCSSTSCPSDYYYGMDVW | 243 | 43 | None | | 13295 |
| CARDLSYSSSWYRYFDYW | 567 | 43 | None | | 12499 |
| CARDLGYCTSTSCY-SFDYW | 421 | 43 | None | | 18870 |
| AFDILTGYYGVYYFDYW | 456 | 42 | None | | 69798 |
| YCSGGSCYSPAFDIW | 399 | 42 | None | | 69799 |
| CASGSGSYYNENWFDPW | 268 | 42 | None | | 14550 |
| CARRRSSGYSPFDYW | 499 | 41 | None | | 20566 |
| CALPICFLMCCWIWFASILLRVFALMFIKDIGLKFSL | 107 | 41 | None | | 69800 |
| FVVSLPGF | | | | | |
| CATTDYGDYEDAFDIW | 213 | 41 | None | | 12864 |
| CALPIWISFLMCCWIQFASILLRIFILMFIRDIGLKF | 107 | 41 | None | | 69801 |
| SFFVVSLSGF | | | | | |
| CALPICFLMYCWIRFASILLRIFALMFIRDIGLKFSL | 107 | 41 | None | | 69802 |
| FVVCLPGF | | | | | |
| CALPIWISFLICCWIRFASILLRIFASMFIKDIGLKFS | 107 | 41 | None | | 69803 |
| FLVVSLPGF | | | | | |
| CALPIFLMCCWIRFASILLRIFTSMFIRDIGLKFSFFV | 107 | 41 | None | | 69804 |
| VSLPGF | | | | | |
| CALPIYFLMCCWIRFASILLRIFASMFIRDIGLEFSFF | 107 | 41 | None | | 69805 |
| VVSLPGF | | | | | |
| CALPIWISFLMCCWIWFASILLRIFALMFIRYTGLKFS | 107 | 41 | None | | 69806 |
| FIVLSLPGF | | | | | |
| CARDTRTYYDILTGYYPSGYFDYW | 265 | 41 | None | | 18062 |
| CALPICFLMYCWICFASILLRIFALMFIRDIGLKFSFF | 107 | 41 | None | | 69624 |
| VVSLPGF | | | | | |
| CVAGGNRFFDYW | 203 | 41 | None | | 18579 |
| CSSDLFLMCCWIRFASILLRIFASMFIKDIGLKFSFFV | 107 | 41 | None | | 69807 |
| VSLPGF | | | | | |
| CALPIWISFLMCWWIQFASILLKIFASIFIRDIGLKF | 104 | 40 | None | | 69808 |
| SFFVVSLPGF | | | | | |
| CALPIFLMCCWIQFASILLRIFASMFIRDIGLKFSFFV | 105 | 40 | None | | 69809 |
| LSLSGF | | | | | |
| CALPIWISFLTCCWIRFASILLRIFASMFIREIGLKF- | 105 | 40 | None | | 69810 |
| FFCLSLPGF | | | | | |
| CSSTSCYWGYMDVW | 256 | 40 | None | | 13181 |
| CALDSGYSSSWYGVFDYW | 287 | 40 | None | | 14233 |
| CARDLGSSGHYYYYMDVW | 213 | 40 | None | | 18363 |
| CSSDLFLMCCWIWFASILLRIFASMFIKDIGLKCSFFV | 104 | 40 | None | | 69811 |
| VSLPGF | | | | | |
| CALPI-LSFLMCCWIQFASILLRIFALMFMKDTGLKFSF | 104 | 40 | None | | 69812 |
| VVVSLPGF | | | | | |
| CALPIWISFFMCCCIQFASILLRIFALMFIRDIGLKLS | 105 | 40 | None | | 69813 |
| FFVVSLPGF | | | | | |
| CALPIWISFLICCWIQFPSILLRIFASMFIRDIGLKFS | 104 | 40 | None | | 69814 |
| SFVVSLPGF | | | | | |
| CAKDRRGYVD | 320 | 39 | None | | 69815 |
| CALPI--CFLMCCWIQFASILLMIFTSMFIRDIGLKFS | 102 | 39 | None | | 69816 |
| FFVVSLSGF | | | | | |
| CARSNYDILTGYYTPWDYW | 680 | 39 | None | | 16943 |
| CARDRGYMDVW | 317 | 38 | None | | 8567 |
| CSSDL--LLTFCWIWFASILLRIFASMFIRDIGLKFSF | 98 | 38 | None | | 69817 |
| SVVFLAGF | | | | | |
| CARGNCSGGSCYSSPFDIW | 558 | 38 | None | | 23204 |
| CARDRPH-YYDSSGPTPFDYW | 923 | 38 | None | | 9147 |
| TMTNDAFDIW | 633 | 38 | None | | 69818 |
| GSSSSSSGWFDPW | 280 | 38 | None | | 69819 |
| CARATRYCGGDCYP-DYW | 217 | 37 | None | | 9242 |
| CARGVSSGWTEFDYW | 583 | 37 | None | | 14008 |
| CARDQSSGWYSNWFDPW | 389 | 37 | None | | 6057 |
| AVLYCSSTSCYATNYYYYGMDVW | 218 | 37 | None | | 69820 |
| CAREFGYSSGWYAYFDYW | 325 | 37 | None | | 16126 |
| CARLHDSSGYYFGFDYW | 525 | 37 | None | | 6046 |
| CALPIFLMCCWIQSASILLRIFTLMFIRGIGLTFSFF | 97 | 37 | None | | 69821 |
| VVSLPGF | | | | | |
| CARDTPFHHYYDSSGYFDYW | 1023 | 37 | None | | 22769 |
| CARGLSGY--DDAFDLW | 694 | 36 | None | | 22167 |
| CARDTAYCSGGSCYSGDYYYGMDVW | 264 | 36 | None | | 15499 |
| LDKLLTCCWIWFASILLRIFASMFIRDIGLKFSFSVV | 93 | 36 | None | | 69822 |
| FLAGF | | | | | |
| CARDPETSYYDSSGPIDYW | 200 | 36 | None | | 16162 |
| VRGYSSPDYW | 482 | 36 | None | | 69823 |
| CARVNNPFYYYYAMDVW | 282 | 36 | None | | 5661 |
| CALPIWISFLMRCWNWFASILLRIFTSILFRDIGLK | 92 | 35 | None | | 69824 |
| FSFSVVSLPGF | | | | | |
| CARGYCSGGSCYGIYYYYYYMDVW | 218 | 35 | None | | 19175 |
| CARDLYSSGWYGQPDYW | 239 | 35 | None | | 19513 |
| CARALEGYSSSWYVDYW | 2588 | 35 | None | | 13920 |
| CALPIWMDKLLTCCWIWFASILLRIFASMFIRDIGLK | 91 | 35 | None | | 69825 |
| FSFSVVFLAGF | | | | | |
| CVRVDWPYYYYYYMDVW | 191 | 35 | None | | 9371 |
| CARAPYDILTGYVEYYFDYW | 390 | 35 | None | | 12880 |
| CARDTMVTFL-YYYYYGMDVW | 384 | 34 | None | | 12719 |
| CARDQ-IAVAGTSDYW | 1074 | 34 | None | | 22432 |
| RPGPCSGGSCYYDFDYW | 206 | 34 | None | | 69826 |
| CAR-HLDILTGYYNYYFDYW | 237 | 34 | None | | 21159 |
| CAREVCGGDCYP | 237 | 34 | None | | 69827 |
| CARTLSGYDSDDAFDIW | 185 | 34 | None | | 21212 |
| CARVQLVGYYYYMDVW | 170 | 34 | None | | 9132 |
| CTTVDDYGDYYY-MDVW | 189 | 33 | None | | 18462 |
| LSLLGGWFDPW | 194 | 32 | None | | 69828 |
| CASAGYSS-SRWFDPW | 284 | 32 | None | | 12723 |
| FLMRCWNWFASILLRIFTSILFRDIGLKFSFSVVSLPGF | 84 | 32 | None | | 69829 |
| CARGDGYFEYW | 304 | 32 | None | | 22970 |
| CAKDSVRGVGWFDPW | 1274 | 32 | None | | 20314 |
| CARGGYSYGYLLDYW | 195 | 31 | None | | 8659 |
| CARDKTYCSSGSCYSYDYYYGMDVW | 221 | 31 | None | | 19050 |
| CARGLEGYSSSWYIDYW | 621 | 31 | None | | 8456 |
| CARVNNGGYYFDYW | 1083 | 31 | None | | 12629 |
| CARDNSGSFDIW | 184 | 31 | None | | 14812 |
| CARADYDSSG -- RQTFDYW | 395 | 30 | None | | 16275 |
| SGSQSRFNYW | 103 | 30 | None | | 69830 |
| CAK -- DPQRRYYYDSSGY | 179 | 30 | None | | 69831 |
| YDYVGVFDYW | 234 | 30 | None | | 69832 |
| CALPIWISFLMCCCIRFASISLRIFALMFIGHIGLKF | 78 | 30 | None | | 69833 |
| SFFVLFMPGF | | | | | |
| CARTPRYCGGDCLDYW | 116 | 30 | None | | 20061 |
| CSSDLFLMCCGIQFASILLRIFTLKFIRDTGLMFSFF | 71 | 29 | None | | 69834 |
| VLSLPGF | | | | | |
| SGYSLNNWFDPW | 432 | 29 | None | | 69835 |
| CARGY--SYGPYWYFDLW | 163 | 29 | None | | 16003 |
| CARGGYGGGLFDYW | 442 | 28 | None | | 19745 |
| CASWNYD-LTGDYYYYMDVW | 287 | 28 | None | | 15528 |
| CALPILNFLMCCQIQFAGILLRTFASMFIRGIDLKFS | 71 | 28 | None | | 69836 |
| FFVMSLPGF | | | | | |
| CARHRYSSSSYYFDYW | 426 | 28 | None | | 23265 |
| CAKADCSSTSCRNWFDPW | 251 | 28 | None | | 13972 |
| CARSTTSYCSSTSC--YFDYW | 183 | 27 | None | | 19842 |
| LSTGWYTTFDYW | 233 | 27 | None | | 69837 |
| ARGDGYYSPFDYW | 265 | 27 | None | | 69838 |
| CAKD--GSSWFGWFDPW | 154 | 27 | None | | 15755 |
| RSLGYCSSTSCFNFDYW | 234 | 26 | None | | 69839 |
| CAHRRSGWT--SGWFDPW | 229 | 26 | None | | 16243 |
| CARMVGQYCSSTSCYNYFDYW | 154 | 26 | None | | 5675 |
| CART--YCTGGSCYFRAFDYW | 318 | 26 | None | | 5484 |
| WIQLWSSYFDYW | 146 | 26 | None | | 69840 |
| CAYAGGPHYYMDVW | 131 | 26 | None | | 15692 |
| CARHSGSGWYSPFDYW | 274 | 25 | None | | 5605 |
| CARMGSCGGGSCYSGYFDPW | 95 | 25 | None | | 17213 |
| CATIGGYSYGHDAFDIW | 134 | 24 | None | | 13434 |
| CAREPLSGWYVYYW | 292 | 24 | None | | 13886 |
| GQNGAFDIW | 661 | 24 | None | | 69841 |
| CARGYCAAGSCYSV | 213 | 24 | None | | 69842 |
| RAVAGHIDYW | 886 | 24 | None | | 69843 |
| CASLTGYSSSWYVGDYW | 224 | 24 | None | | 21794 |
| AASYCGGDCYSDAFDIW | 140 | 23 | None | | 69844 |
| CAAL-SSSWLLDYW | 316 | 23 | None | | 23418 |
| CARGTAAIVNWFDPW | 96 | 23 | None | | 11814 |
| CAR-HFGSGNYCFDYW | 783 | 23 | None | | 23281 |
| CALPI---FLMCCCIRFASISLRIFALMFIGHIGLKFS | 57 | 23 | None | | 69845 |
| FFVLFMPGF | | | | | |
| CQQFNNF | 1573 | 23 | None | | 23139 |
| CARAGYCTGSSCYD-AFDIW | 149 | 23 | None | | 9501 |
| CARGNFGYSSSWYMLDYW | 196 | 22 | None | | 20776 |
| CAKDPGYSYGYGGFDYW | 125 | 22 | None | | 12557 |
| RSARGYCSSTSCY-AFDIW | 178 | 22 | None | | 69846 |
| CAKADCSSTSCHNQFDPW | 166 | 21 | None | | 20013 |
| CARSYSGSYRDFFDYW | 285 | 21 | None | | 23144 |
| CAR-RLSRGWYDWFDPW | 183 | 21 | None | | 5685 |
| CATVVGATGAFDIW | 115 | 21 | None | | 6068 |
| CARELRFCSGGSCY-PDDWFDPW | 73 | 20 | None | | 22562 |
| CQQRTHWPGPF | 9115 | 20 | None | | 23060 |
| CARDRATSGSYSYYFDYW | 129 | 20 | None | | 18442 |
| CARDKGGSKWYFDLW | 79 | 19 | None | | 22773 |
| SASSNGFDPW | 266 | 19 | None | | 69847 |
| CARAGHCSGGSCFPVPDYW | 123 | 19 | None | | 21356 |
| CYSTDSSGNH | 7487 | 18 | None | | 69848 |
| CARDKNYYGSGSRDAFDIW | 110 | 18 | None | | 23055 |
| CQSADISGTYPPVLF | 61523 | 18 | None | | 13257 |
| CARGGYSSSSGWFDPW | 79 | 18 | None | | 17482 |
| ARGYYDLSAGFDYW | 171 | 18 | None | | 69849 |
| CARGVIGWYYFDFW | 194 | 18 | None | | 17271 |
| CMQGTHWPLTF | 14260 | 18 | None | | 19150 |
| CARRGSG-YRDFFDSW | 139 | 17 | None | | 23171 |
| CMQGTHFYSF | 7558 | 17 | None | | 5750 |
| CARDGGYTYGSARFDYW | 112 | 17 | None | | 3960 |
| CAR-GSGSYYYDSSFDYW | 121 | 17 | None | | 14467 |
| CARMWVDCSGGSCFYWFDPW | 50 | 16 | None | | 13435 |
| CAAAGDSGGYQKVTF | 70 | 16 | None | | 69850 |
| CGTWDNSLSGGVF | 3002 | 16 | None | | 23383 |
| CATGGGSYFGHNWFDPW | 100 | 15 | None | | 13601 |
| CARVQSVDTAMDYYYGMDVW | 85 | 14 | None | | 22566 |
| CARDNVYRDYMDVW | 84 | 14 | None | | 23121 |
| CARGPGLVT-SNWFDPW | 173 | 14 | None | | 5436 |
| CATWDDSLTGCWLF | 10637 | 14 | None | | 12686 |
| GTSNTGKLIF | 50 | 14 | None | | 69851 |
| CAANSGYSTLTF | 92 | 14 | None | | 69852 |
| AARSGNTGKLIF | 47 | 14 | None | | 69853 |
| CAKFDRRDDYGDHFDYW | 193 | 13 | None | | 22452 |
| CQSADSDATY | 11465 | 13 | None | | 69854 |
| CARLGYCSGALCYRADYYYGMDVW | 155 | 13 | None | | 18286 |
| CARDRGIDYGDQFDYW | 176 | 13 | None | | 5586 |
| CAGDGYSSASKIIF | 68 | 13 | None | | 69855 |
| CAVWDDSLSGRNVVF | 10728 | 13 | None | | 19781 |
| CMQATHWPALS | 12318 | 13 | None | | 69856 |
| CAAWDNSLSGR | 10739 | 13 | None | | 69857 |
| CQSADITGTYVLF | 625 | 12 | None | | 19086 |
| CSSDLYQHRTMQAHELFRYFRMPELVDFRQYVRTLPT | 62 | 12 | None | | 69858 |
| NTLMGF | | | | | |
| CALPILENYQHRTMQAHELFRYFRMPELVDFRQYVRTL | 62 | 12 | None | | 69859 |
| PTNTLMGF | | | | | |
| CALPGYSSASKIIF | 64 | 12 | None | | 69860 |
| CAVGGSSASKIIF | 40 | 12 | None | | 69861 |
| CAKELHTSGWYFDYW | 257 | 12 | None | | 23179 |
| CALPIYQHRTMQAHELFRYFRMPELVDFRQYVRTLPT | 62 | 12 | None | | 69862 |
| NTLMGF | | | | | |
| CGTWDTSLSVGVF | 2988 | 12 | None | | 8579 |
| CAVAYSSASKIIF | 40 | 12 | None | | 69863 |
| CAASSGSGNTGKLIF | 42 | 11 | None | | 69864 |
| CGTWDSSLTPG | 2959 | 11 | None | | 69865 |
| COSADITGTYVIF | 620 | 11 | None | | 23167 |
| CDSSVTERNPSWHSAPALRAFVSRLCMRVTRARAPSF | 41 | 11 | None | | 69866 |
| PRAKEDGF | | | | | |
| CALPICKLCNVTEHIESVVIVVLLSSLSPVSCYTYSV | 50 | 11 | None | | 69867 |
| PNCKLNF | | | | | |
| CALPIWNPSWHSAPALRAFVSRLCMRVTRARAPSFPR | 41 | 11 | None | | 69868 |
| AKEDGF | | | | | |
| CALPISERNPSWHSAPALRAFVSRLCMRVTRARAPSF | 41 | 11 | None | | 69869 |
| PRAKEDGF | | | | | |
| CAATYSSASKIIF | 38 | 11 | None | | 69870 |
| CARDMSGDWNWYFDLW | 42 | 10 | None | | 18523 |
| CSSDLQTSSLESCIYMWLDKYLKEKNTKRDSPHSLPP | 94 | 10 | None | | 69871 |
| PTIFFNF | | | | | |
| CGTWDTSLNANWVF | 2956 | 10 | None | | 17640 |
| CSSDLLEGASLPHPEGGPAHHPLLPGVSQPPSPRGEK | 31 | 9 | None | | 69872 |
| LVGEGWLGF | | | | | |
| CQVWHSSGDHPQLVF | 300 | 9 | None | | 8459 |
| CSSDL--GASLPHPEGGPAHHPLLPGVSQPPSPRGEKL | 31 | 9 | None | | 69873 |
| VGEGWLGF | | | | | |
| CALPIWSLEGASLPHPEGGPAHHPLLPGVSQPPSPRG | 31 | 9 | None | | 69874 |
| EKLVGEGWLGF | | | | | |
| CMHGTHWPPRYTF | 3465 | 9 | None | | 23422 |
| CVVRDTGNQFYF | 30 | 9 | None | | 69875 |
| CVRDQRRGYSGYDLDYW | 52 | 9 | None | | 22976 |
| CAVSDLYSSASKIIF | 33 | 9 | None | | 69876 |
| CAVSFYSSASKIIF | 34 | 9 | None | | 69877 |
| CAVRGDSSASKIIF | 32 | 9 | None | | 69878 |
| CSSDLWASLFNQTVFLGLQGPLTPDGHRVPPADHRAW | 162 | 8 | None | | 69879 |
| LSPEGGF | | | | | |
| PCKARLCKVSPLQTEWFKTARMHSSIYNSKAPCTELGF | 61 | 8 | None | | 69880 |
| CALPISRIPTSARGPWWFCGVSGSVLRVGRATKLVLP | 112 | 8 | None | | 69881 |
| SSFQFVF | | | | | |
| CARGYCGGDCYWGS--DAFDWW | 21 | 8 | None | | 5595 |
| CALPISGMEQNRKDPPGPATTSLAPVRGGTLCIGPGS | 32 | 8 | None | | 69882 |
| SSCRQRPRGF | | | | | |
| CSSDLQNRKDPPGPATTSLAPVRGGTLCIGPGSSSCR | 32 | 8 | None | | 69883 |
| QRPRGF | | | | | |
| CSSDLSRIPTSARGPWWFCGVSGSVLRVGRATKLVL | 112 | 8 | None | | 69884 |
| PSSFQFVF | | | | | |
| CALPI-WMEQNRKDPPGPETTSLAPVRGGTLCIGPGSSS | 32 | 8 | None | | 69885 |
| CRQRPRGF | | | | | |
| CALPISGMEQNRKDPPGPETTSLAPVRGGTLCIGPGS | 32 | 8 | None | | 69886 |
| SSCRQRPRGF | | | | | |
| MEQNRKDPPGPATTSLAPVRGGTLCIGPGSSSCRQRPR | 32 | 8 | None | | 69887 |
| GF | | | | | |
| CYLTDSSINHRV | 7359 | 7 | None | | 69888 |
| CALPIC--VGLSLLPVPSHLVHEPRSWSLVLEAAVMS | 36 | 7 | None | | 69889 |
| GTGICLAF | | | | | |
| CALPISPAYIIMSFSLITHFTANETQARRTERWPSSH | 21 | 7 | None | | 69890 |
| RNRTSWDSCF | | | | | |
| CLLKTTVLSFMQCFCTETAVSCQCTVNGRKTNKMSFQV | 75 | 7 | None | | 69891 |
| FLRDGF | | | | | |
| CALPISGFYYTGLYSEIFFIILLTIDRYLAIVHAVFAL | 34 | 7 | None | | 69892 |
| RARTVTF | | | | | |
| CAASYSSNTGKLIF | 32 | 7 | None | | 69893 |
| CALPIWGLGVGLSLLPVPSHLVHEPRSWSIVLEAAVM | 36 | 7 | None | | 69894 |
| SGTGICLAF | | | | | |
| CALPILGVGLSLLPVPSHLVHEPRSWSLVLEAAVMSG | 36 | 7 | None | | 69895 |
| TGICLAF | | | | | |
| CALPICLGVGLSLLPVPSHLVHEPRSWSLVLEAAVMS | 36 | 7 | None | | 69896 |
| GTGICLAF | | | | | |
| CALPIYIIMSFSLITHFTANETQARRTERWPSSHRN | 21 | 7 | None | | 69897 |
| RTSWDSCF | | | | | |
| CALPISYIIMSFSLITHFTANETQARRTERWPSSHR | 21 | 7 | None | | 69898 |
| NRTSWDSCF | | | | | |
| CALPIAYIIMSFSLITHFTANETQARRTERWPSSHR | 21 | 7 | None | | 69899 |
| NRTSWDSCF | | | | | |
| CAKGDCSGGSCYDY 915 81 | None | None | None | | 69766 |

LENGTHY TABLES
The patent application contains a lengthy table section. A copy of the table is available in electronic form from the USPTO web site (https://seqdata.uspto.gov/?pageRequest=docDetail&DocID=US20250034234A1). An electronic copy of the table will also be available from the USPTO upon request and payment of the fee set forth in 37 CFR 1.19(b)(3).

1. A method for treating COVID-19 in patients, comprising the steps of:
(a) identifying antibodies produced by COVID-19 patients, using a structural classification of COVID-19 antibodies to categorize potential efficacious antibodies;
(b) identifying patients who lack certain types of antibodies to COVID-19;
(c) identifying critical intervention time in the patients who lack the certain types of antibodies to COVID-19 patients when the patients have reduced levels of COVID-19 antibodies; and
(d) administering to the patients a therapy that is efficacious for treating COVID-19 patients.

2. The method of claim 1, wherein the antibodies are generated by patients that survived severe COVID-19.

3. A method for treating COVID-19 patients, comprising the step of administering to the COVID-19 patient a therapeutic antibody comprising one or more antibodies from TABLE 6.

4. A method for treating COVID-19 patients, comprising the step of administering to the COVID-19 patient a therapeutic antibody comprising one or more antibodies from TABLE A4.

5. A method of testing for the presence in a patient of bacteria that can cause bacteremia using a direct from blood, without the need for culture, reverse transcriptase polymerase chain reaction (RT-qPCR) assay, comprising the steps of:
(a) obtaining RNA identified from patients with bacteremia;
(b) identifying the bacteria correlating with the obtained RNA; and
(c) identifying the presence of bacteria in bacteremia patients using the RT-qPCR) assay.

6. The method of claim 5, wherein the bacteria are selected from the group consisting of Staphylococcus aureus, Escherichia coli, and Pseudomonas aeruginosa.

7. The method of claim 5, further comprising the step of validating the RT-qPCR assays in samples from patients with and without bacteremia.

8. A method of testing for the presence in a patient of bacteria that can cause pneumonia using a direct from blood, without the need for culture, reverse transcriptase polymerase chain reaction (RT-qPCR) assay, comprising the steps of:
(a) obtaining RNA identified from patients with pneumonia;
(b) identifying the bacteria correlating with the obtained RNA; and
(c) identifying the presence of bacteria in pneumonia patients using the RT-qPCR) assay.

9. The method of claim 8, wherein the bacteria are selected from the group consisting of Staphylococcus aureus, Pseudomonas aeruginosa, and Haemophilus influenzae.

10. The method of claim 8, further comprising the step of validating the RT-qPCR assays in samples from patients with and without pneumonia.

11. A method of testing for the presence in a patient of bacteria that contain expressed resistance genes that would influence antibacterial treatment using a direct from blood, without the need for culture, reverse transcriptase polymerase chain reaction (RT-qPCR) assay, comprising the steps of:
(a) obtaining RNA identified from patients with a bacterial infection;
(b) identifying the expressed resistance genes correlating with the obtained RNA; and
(c) identifying the presence of bacteria that contain expressed resistance genes in patients using the RT-qPCR) assay.

12. The method of claim 11, wherein the bacteria are selected from the group consisting of Staphylococcus aureus, Escherichia coli, Pseudomonas aeruginosa, and Haemophilus influenzae.

13. The method of claim 11, further comprising the step of validating the RT-qPCR assays for resistance genes in samples from patients with and without infections.
